(19) United States
(12) Patent Application Publication
(10) Pub. No.: US 2021/0353648 A1
(43) Pub. Date: Nov. 18, 2021

(54) GRAPIPRANT UNIT DOSAGE FORMS

(71) Applicants: Arrys Therapeutics, Inc., Boston, MA (US); AskAt Inc., Nagoya-Shi (JP)

(72) Inventors: Mark MANFREDI, Newton, MA (US); Jeffrey ECSEDY, Newton, MA (US); Atsushi NAGAHISA, Nagoya-shi (JP); Takako OKUMURA, Nagoya-shi (JP)

(21) Appl. No.: 17/278,601

(22) PCT Filed: Sep. 27, 2019
(86) PCT No.: PCT/US19/53413

§ 371 Date: Mar. 22, 2021

Related U.S. Application Data
(60) Provisional application No. 62/737,285, filed on Sep. 27, 2018.
(60) Provisional application No. 62/834,513, filed on Apr. 16, 2019.

(51) Int. Cl. A61K 31/64; A61K 9/20

(52) U.S. Cl. CPC A61K 31/64; A61K 9/2054; A61K 9/2036; A61K 9/2013; A61K 9/2009; A61K 9/2018

(57) ABSTRACT

The present invention provides grapiprant unit dosage forms, and methods of use thereof for treating a proliferative disorder.

### TECHNICAL FIELD OF THE INVENTION

The present invention relates to grapiprant unit dosage forms and methods of use thereof.

### BACKGROUND OF THE INVENTION

Prostaglandins are mediators of pain, fever and other symptoms associated with inflammation. Prostaglandin E2 (PGE2) is the predominant eicosanoid detected in inflammation conditions. In addition, it is also involved in various physiological and/or pathological conditions such as hyperalgesia, uterine contraction, digestive peristalsis, awakeness, suppression of gastric acid secretion, blood pressure, platelet function, bone metabolism, angiogenesis or the like.

Four PGE2 receptor subtypes (EP1, EP2, EP3 and EP4) displaying different pharmacological properties exist. The EP4 subtype, a Gs-coupled receptor, stimulates cAMP production as well as PI3K and GSK3β signaling, and is distributed in a wide variety of tissue suggesting a major role in PGE2-mediated biological events. Various EP4 inhibitors, including N-((4-(2-ethyl-4,6-dimethyl-1H-imidazo[4,5-c]pyridin-1-yl)phenethyl)carbamoyl)-4-methylbenzenesulfonamide (also known as “grapiprant”), have been described previously, for example, in WO 2002/032900, WO 2005/021508, U.S. Pat. Nos. 6,710,054, and 7,238,714, the contents of which are incorporated herein by reference in their entireties.

### SUMMARY OF THE INVENTION

It has been found that the grapiprant unit dosage forms of the invention are suitable for oral administration in a patient for treating a proliferative disorder. Accordingly, in one aspect, the present invention provides a unit dosage form of a pharmaceutical composition comprising grapiprant, or a pharmaceutically acceptable salt thereof. In some embodiments, a unit dosage form of the invention comprises about 50 mg to about 375 mg grapiprant, or a pharmaceutically acceptable salt thereof. In some embodiments, a unit dosage form of the invention comprises one or more pharmaceutically acceptable excipient or carrier. In some embodiments, one or more pharmaceutically acceptable excipient or carrier comprises microcrystalline cellulose, lactose monohydrate (modified), croscarmellose sodium, hydroxypropyl cellulose, and magnesium stearate. In some embodiments, a unit dosage form of the invention exhibits pharmacokinetics results as described herein.

In some embodiments, the present invention provides a unit dosage form of a pharmaceutical composition, in oral-powder-for-constitution (OPC) formulation, comprising about 1 mg to about 2000 mg grapiprant, or a pharmaceutically acceptable salt thereof. In some embodiments, the present invention provides a unit dosage form of a pharmaceutical composition, in liquid form, comprising about 1 mg to about 2000 mg grapiprant, or a pharmaceutically acceptable salt thereof, and water.

In another aspect, the present invention provides a method for treating a proliferative disorder, comprising administering a grapiprant unit dosage form, as described herein. In some embodiments, a proliferative disorder is as described herein.

### BRIEF DESCRIPTION OF FIGURES

FIG. 1 depicts mean serum grapiprant concentration vs. time profiles following oral administration of 375 mg tablets of grapiprant in healthy subjects under fasted and fed conditions (N=12).

FIG. 2 depicts Growth Kinetics in BALB/C Mice Bearing CT-26 Tumors. BALB/C mice bearing CT-26 tumors were treated with vehicle (0.5% methylcellulose and IgG2a), anti-PD-1, or Compound B at 15 mg/kg QD and BID alone or in combination with anti-PD-1. Mean tumor volumes (mm³) and standard error of the mean (n=10/group) are shown.

FIG. 3 depicts Kaplan-Meier Curve of Tumor Bearing Mice. Kaplan-Meier curve of tumor-bearing mice treated with vehicle (0.5% methylcellulose and IgG2a), anti-PD-1, or Compound B at 15 mg/kg QD and BID alone or in combination with anti-PD-1. Mice were monitored out 99 days after tumor inoculation, and animals were sacrificed when tumor sizes exceeded 3000 mm³.

FIG. 4 depicts Tumor Growth Kinetics in BALB/C Mice Bearing 4T1 Tumors. BALB/C mice bearing 4T1 tumors were treated with vehicle, anti-CTLA4, or Compound B at 15 mg/kg BID alone or in combination with anti-CTLA41. Mean tumor volumes (mm³) and standard error of the mean (n=10/group) are shown.

FIG. 5 depicts Kaplan-Meier Curve of Tumor-Bearing Mice Study. Kaplan-Meier curve of tumor-bearing mice treated with vehicle, anti-CTLA4, or Compound B at 15 mg/kg BID alone or in combination with anti-CTLA4. Mice were monitored for 41 days after tumor inoculation, and animals were sacrificed when tumor sizes exceeded 3000 mm³.

FIG. 6 depicts mean (SD) grapiprant serum concentrations following single oral doses of grapiprant OPC in the fasted state to healthy adult subjects.

FIG. 7 depicts individual and mean dose-normalized* AUC(Tlast) values following single 1 mg, 3 mg, 10 mg, 30 mg, 100 mg, 300 mg, 600 mg, 1000 mg, 1500 mg, and 2000 mg doses of grapiprant OPC in the fasted state to healthy adult subjects.

FIG. 8 depicts BALB/C mice bearing CT-26 tumors treated with vehicle (0.5% methylcellulose and PBS), anti-PD1, or Compound B at 15 mg/kg BID alone or in combination with anti-PD1. Mean tumor volumes (mm³) and standard error of the mean (n=7/group) are shown.

FIG. 9 depicts BALB/C mice bearing 4T1 tumors treated with vehicle (0.5% methylcellulose and PBS), anti-PD1, or Compound B at 15 mg/kg BID alone or in combination with anti-PD1. Mean tumor volumes (mm³) and standard error of the mean (n=7/group) are shown.

FIG. 10 depicts the immune cell composition of CT-26 tumors grown in BALB/c mice treated with vehicle (0.5% methylcellulose and PBS), anti-PD1, or Compound B (CPD-B) at 15 mg/kg BID alone or in combination with anti-PD1. The percentage of regulatory T cells (a), dendritic cells (b), activated T cells (c) and activated PD-1 high T cells (d) is shown. p values determined using a Student's T-test comparing vehicle to treated groups; *p<0.05, **p<0.01.

### DETAILED DESCRIPTION OF THE INVENTION

### 1. General Description of Certain Embodiments of the Invention

Various dosage levels of grapiprant tablets have been administered in healthy adult subjects via a 14 day oral administration regimen, and it was found that multiple-dose administration of grapiprant for 14 days was well tolerated at doses up to about 300 mg BID (twice a day). Systemic exposure increased with dose in an approximately dose proportional manner between 50 and 150 mg BID. However, when the dose was increased 2-fold from 150 mg BID to 300 mg BID, mean exposures increased approximately 3-fold. It was also found that a 375-mg dose of grapiprant tablets was well-tolerated under both fasted and fed conditions in healthy adult subjects.

It was also found that a 17 day oral administration of grapiprant at about 250 mg BID was well tolerated in elderly subjects with mild renal impairment. The mean exposure in this group of subjects was slightly greater than the mean exposure observed with the 300 mg BID dose in healthy adults, with considerable overlap in individual exposures between these two groups.

Accordingly, in some embodiments, the present invention provides a unit dosage form of a pharmaceutical composition comprising about 50 mg to about 375 mg grapiprant, or a pharmaceutically acceptable salt thereof, and one or more pharmaceutically acceptable excipient or carrier.

In addition, it has been found that, subsequent to a single, oral dose of about 1 mg to about 2000 mg grapiprant in OPC formulation, grapiprant exhibited rapid absorption followed by a bi-exponential decline in serum concentration, and that the exposures and peak serum concentrations of grapiprant increased with dose. No positive results for fecal occult blood tests, no abnormalities in cardiac telemetry, and no clinically important, treatment-related changes in vital signs measurements or ECG parameters were observed for any treatment groups.

Accordingly, in some embodiments, the present invention provides a unit dosage form of a pharmaceutical composition, in OPC formulation, comprising about 1 mg to about 2000 mg grapiprant, or a pharmaceutically acceptable salt thereof. In some embodiments, the present invention provides a liquid unit dosage form of a pharmaceutical composition comprising about 1 mg to about 2000 mg grapiprant, or a pharmaceutically acceptable salt thereof, and water.

In some embodiments, the present invention provides a method for treating a proliferative disorder, comprising administering a grapiprant unit dosage form, as described herein.

### 2. Definitions

As used herein, the term “grapiprant” refers to a compound of formula:

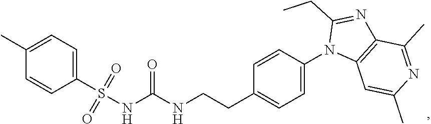

In some embodiment, grapiprant, or a pharmaceutically acceptable salt thereof, is in crystal form. In some embodiments, grapiprant, or a pharmaceutically acceptable salt thereof, is in polymorph Form A, as described in U.S. Pat. Nos. 7,960,407 and 9,265,756, the contents of which are incorporated herein by reference in their entireties. In some embodiments, polymorph Form A of grapiprant, or a pharmaceutically acceptable salt thereof, is characterised by a powder X-ray diffraction pattern obtained by irradiation with Cu Kα radiation which includes main peaks at 2-Theta° 9.8, 13.2, 13.4, 13.7, 14.1, 17.5, 19.0, 21.6, 24.0 and 25.7+/−0.2. In some embodiments, polymorph Form A of grapiprant, or a pharmaceutically acceptable salt thereof, is characterised by differential scanning calorimetry (DSC) in which it exhibits an endothermic event at about 160° C. In some embodiments, polymorph Form A of grapiprant, or a pharmaceutically acceptable salt thereof, exhibits an X-ray powder diffraction pattern having characteristic peaks expressed in degrees 2-theta at about 9.9, about 13.5, about 14.3, about 16.1, about 17.7, about 21.8, about 24.14, and about 25.8. In some embodiments, polymorph Form A of grapiprant, or a pharmaceutically acceptable salt thereof, exhibits a differential scanning calorimetry profile having showed an endotherm/exotherm at about 155-170° C. In some embodiments, polymorph Form A of grapiprant, or a pharmaceutically acceptable salt thereof, exhibits a thermogravimetric analysis showing a loss of mass of 0.5-0.6% when heated from about 30° to about 150° C.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention.

Serum pharmacokinetic parameters can be computed using WinNonlin Version 3.2 (Pharsight®, Mountain View, Calif.) using standard non-compartmental methods. As used herein, the term “Cmax” refers to the maximum observed serum concentration of grapiprant estimated directly from concentration versus time data. As used herein, the term “Tmax” refers to the time of the first occurrence of Cmax. As used herein, the term “AUC(0-tlast)” refers to the area under the serum concentration vs. time curve from time 0 to Tₗₐₛₜ (the time of the last quantifiable concentration estimated using the linear/log trapezoidal approximation). When data permitted, the terminal elimination rate constant (kel) was estimated using linear least-square regression analysis of the serum concentration-time data obtained during the terminal log-linear phase. As used herein, the term “AUC(0-inf)” refers to AUC(0-tlast) extrapolated to infinity, which is calculated as the sum of AUC(0-tlast) and Cest/kel, wherein Cest is the predicted plasma concentration obtained from the log linear regression analysis at the last quantifiable time point. As used herein, the term “t½” refers to terminal phase half-life calculated as ln(2)/kel. As used herein, the term “AUC(last-inf)” refers to the area under the serum concentration versus time curve from Tₗₐₛₜ to infinity, which is estimated as Cpest/kel, wherein Cpest represented the estimated concentration at time Tₗₐₛₜ based on the aforementioned regression analysis. As used herein, the term “AUCτ” refers to the area under the serum concentration versus time curve over the dosing interval. As used herein, the term “Rac” refers to the accumulation ratio of grapiprant in the serum, which is calculated as a measure of drug accumulation at steady state compared to Day 1. Rac for the 14 day oral administration described herein is calculated using the equation: Rac=(Day 14 AUCτ/Day 1 AUCτ). Rac for the 17 day oral administration described herein is calculated using the equation: Rac=(Day 17 AUCτ/Day 1 AUCτ). Day 17 is used as steady state in the elderly group of subjects, which is Day 14 of the multiple dosing period for these subjects.

Urine pharmacokinetic parameters can be used to assess the amount of parent drug excreted unchanged in the urine. The urine pharmacokinetic parameters can be calculated under the assumption that the amount of grapiprant excreted unchanged in the urine over the dosing interval was at steady state on the last day of dosing for the 14 day and 17 day oral administration described herein. As used herein, the term “A_{E}” refers to the amount of parent drug excreted from 0-12 hours post dose on day 14 or day 17, calculated by (Urine Grapiprant Concentration×Urine Volume). As used herein, the term “AE/Dose” refers to percent of Dose renally excreted unchanged on day 14 or day 17, calculated by (A_{E}/Dose)×100. As used herein, the term “CLR” refers to renal clearance on day 14 or day 17, calculated by (A_{E}/AUCτ). As used herein, the term “CL_{R}/Fu” refers to renal clearance of unbound drug, calculated by (CLR/Fu), where the fraction of drug in the serum not bound to proteins (Fu) is determined from in vitro human serum protein binding data.

Each bone marker (serum bone-specific alkaline phosphatase and osteocalcin, urinary N-terminal telopeptide of type I collagen) is expressed as percentage change from baseline and analyzed. To assess influence of potential outliers on the bone marker data analyses, the following analysis can be conducted first. Potential outliers are identified as outside an interval consistent with a normal distribution defined as (median±3.5 standard deviations [SD]) for each dosing regimen and day combination. The SD is a pooled estimate across all dosing regimen and day combinations, each estimated by the scaled inter-quartile range 0.75 (Q75-Q25). Explicitly, for each dosing regimen and day combination, its SD denoted by SDi is estimated by its scaled inter-quartile range 0.75 (Q75-Q25). The overall SD is estimated as

$$SD = \sqrt{\frac{1}{15} {\sum }_{i = 1}^{15} {SD}_{i}^{2}} .$$

Then, the (percent change from baseline) values are analyzed with a mixed effect model, with treatment (dosing regimen), day, and treatment-by-day interaction effects considered fixed and subject considered random. Baseline value is not included as a covariate. The model is fit separately to each group of cohorts (Cohorts 1-3 and 5). For Cohorts 1-3, days of interest are day 7 and day 14. For Cohort 5, days of interest are day 10 and day 17. For each group of cohorts, compound symmetry is assumed, and Restricted Maximum Likelihood Estimates are used. The least squares means (LSMeans) and 95% confidence intervals (CI) are obtained for each treatment and day combination. Within each treatment, LSMean percent changes from baseline at each post-dosing day (i.e., day 7 and day 14 for Cohorts 1-3, and day 10 and day 17 for Cohort 5) are obtained, along with their 95% CIs. In addition, for each post-dosing day, differences of the LSMean percent changes from baseline between active dosed regimen and placebo are obtained. The p-values comparing these differences from placebo are obtained using t-tests. No adjustments are made for multiple comparisons. The 95% CIs for the differences are also displayed. If outliers are identified, additional mixed model analyses using the same mixed effect modeling as detailed above are to be conducted excluding the outliers. Descriptive statistics (N, mean, median, SD, coefficient of variation [CV], minimum, and maximum) are provided for each bone marker for both the original data and percentage change from baseline variables by treatment regimen and time point, displayed separately for each group of cohorts. Individual percent-change-from-baseline profile versus time plots are plotted by treatment regimen. Moreover, LSMean treatment regimen profile versus time plots also are provided for each bone marker for the original data expressed as percentage change from baseline, with plots including all data and excluding outliers displayed separately for each group of cohorts.

As used herein, the terms “cohort 1”, “cohort 2”, and “cohort 3” refer to studies where grapiprant tablets are administered at dosage levels of 50 mg, 150 mg, and 300 mg, respectively, in healthy adult subjects with a 14 day oral administration regimen as follows: one dose in the morning in a fasting state on day 1; twice daily (BID, approximately every 12 hours) on days 2 through 13: one dose in a fasting state in the morning, and one dose in the evening on an empty stomach; and one dose in the morning in a fasting state on day 14.

As used herein, the term “cohort 5” and/or “cohort 6” refers to a study where grapiprant tablets are administered at a dosage level of 250 mg in elderly subjects with minor renal impairment with a 17 day oral administration regimen as follows: one dose in the morning in a fasting state on day 1; twice daily (BID, approximately every 12 hours) on days 4 through 16: one dose in a fasting state in the morning, and one dose in the evening on an empty stomach; and one dose in the morning in a fasting state on day 17.

As used herein, the term “healthy adult subjects” refers to subjects between the ages of 18 and 55 years, inclusive, and having no clinically relevant abnormalities identified by a detailed medical history, full physical examination, including blood pressure and heart rate measurement, 12-lead ECG, and clinical laboratory tests. In some embodiments, a healthy adult subject has a Body Mass Index (BMI) from 18 to 30 kg/m², inclusive. In some embodiments, a healthy adult subject has a total body weight>50 kg (110 lbs).

As used herein, the term “elderly subjects with minor renal impairment” refers to subjects 60 years or older, and having a calculated creatinine clearance of approximately 60-80 mL/min derived using the method of Cockcroft and Gault (Cockcroft DW, Gault MH. Prediction of creatinine clearance from serum creatinine. Nephron 1976; 16:31-41, the entire content of which is incorporated herein by reference):

$$Males \text{:} \frac{( 140 - age in years ) \times body weight ( kg ) *}{72 \times serum creatinine ( mg \text{/} dL )}$$

$$Females \text{:} ( 0.85 ) ( calculation for males ) \\ * Weight = lean body weight of the subject .$$

In some embodiments, an elderly subject with minor renal impairment has a Body Mass Index (BMI) from 18 to 30 kg/m², inclusive. In some embodiments, an elderly subject with minor renal impairment has a total body weight>50 kg (110 lbs).

As used herein, the terms “about” or “approximately”, used in conjunction with a numerical value, refer to a range by extending the boundaries above and below the numerical values. For example, the terms “about” or “approximately” can extend the stated value by a variance of 30%, 25%, 20%, 15%, 10%, 9%, 8%, 7%, 6%, 5%, 4%, 3%, 2%, 1%, or 0.5% up and/or down (higher or lower). In some embodiments, the terms “about” or “approximately” extend the stated value by a variance of 25% up and/or down (higher or lower). In some embodiments, the terms “about” or “approximately” extend the stated value by a variance of 10% up and/or down (higher or lower). In some embodiments, the terms “about” or “approximately” extend the stated value by a variance of 5% up and/or down (higher or lower).

As used herein, the terms “fasting state,” “fasting condition,” “fasted state,” or “fasted condition,” refer to a state or condition following at least about 8 hour fast from all food and drink (except water). In some embodiments, a subject is to fast from all food and drink (except water) for at least 8 hours prior to receiving dosing in the morning, and continue to fast until lunch.

As used herein, the terms “fed state” or “fed condition” refer to a state or condition following a standardized high-fat meal. In some embodiments, an administration in a fed state refers to an administration following a standard FDA high-fat breakfast. In some embodiments, subjects receiving the fed regimen fast from all food and drink (except water) for at least 8 hours prior to receiving a test meal. In some embodiments, prior to dosing on Day 1, subjects are served a standard FDA high-fat breakfast composed of 2 eggs fried in butter, 2 strips of bacon, 2 slices of toast with 2 pats of butter, 4 ounces of hash browns and 8 ounces of whole milk, to be ingested and completely consumed within 20 minutes. This breakfast contains approximately 150 protein kcal, 250 carbohydrate kcal, and 500-600 fat kcal. In some embodiments, an alternative meal may be given provided the meal has similar composition and caloric contents.

### 3. Description of Exemplary Embodiments

In one aspect, the present invention provides a unit dosage form of a pharmaceutical composition comprising about 50 mg to about 375 mg grapiprant, or a pharmaceutically acceptable salt thereof, and one or more pharmaceutically acceptable excipient or carrier.

In some embodiments, a unit dosage form of the invention comprises about 50 mg grapiprant, or a pharmaceutically acceptable salt thereof, and one or more pharmaceutically acceptable excipient or carrier (referred to herein as “a 50 mg grapiprant unit dosage form”).

In some embodiments, a 50 mg grapiprant unit dosage form of the invention, upon a 14-day oral administration in healthy adult subjects as described herein, provides one or more pharmacokinetics result on post-dosing day 1 selected from:

a mean serum Cₘₐₓ of about 578 ng/mL;

a mean serum AUC_{τ} of about 3150 ng*h/mL; and

a mean serum Tₘₐₓ of about 1.5 h.

In some embodiments, a 50 mg grapiprant unit dosage form of the invention, upon a 14-day oral administration in healthy adult subjects as described herein, provides a mean serum Cₘₐₓ of about 578 ng/mL on post-dosing day 1. In some embodiments, a 50 mg grapiprant unit dosage form of the invention, upon a 14-day oral administration in healthy adult subjects as described herein, provides a mean serum AUC_{τ} of about 3150 ng*h/mL on post-dosing day 1. In some embodiments, a 50 mg grapiprant unit dosage form of the invention, upon a 14-day oral administration in healthy adult subjects as described herein, provides a mean serum Tₘₐₓ of about 1.5 h on post-dosing day 1.

In some embodiments, a 50 mg grapiprant unit dosage form of the invention, upon a 14-day oral administration in healthy adult subjects as described herein, provides one or more pharmacokinetics result on post-dosing day 14 selected from:

a mean serum Cₘₐₓ of about 611 ng/mL;

a mean serum AUC_{τ} of about 3370 ng*h/mL;

a mean serum Rac of about 1.13;

a mean serum Tₘₐₓ of about 1.25 h;

a mean serum t_{1/2} of about 6.47 h;

a mean urine AE of about 10.6 mg;

a mean urine A_{E}/Dose of about 21.2%;

a mean urine CL_{R} of about 56.3 mL/min; and

a mean urine CL_{R}/Fu of about 6118 mL/min.

In some embodiments, a 50 mg grapiprant unit dosage form of the invention, upon a 14-day oral administration in healthy adult subjects as described herein, provides a mean serum Cₘₐₓ of about 611 ng/mL on post-dosing day 14. In some embodiments, a 50 mg grapiprant unit dosage form of the invention, upon a 14-day oral administration in healthy adult subjects as described herein, provides a mean serum AUC_{τ} of about 3370 ng*h/mL on post-dosing day 14. In some embodiments, a 50 mg grapiprant unit dosage form of the invention, upon a 14-day oral administration in healthy adult subjects as described herein, provides a mean serum R_{ac} of about 1.13 on post-dosing day 14. In some embodiments, a 50 mg grapiprant unit dosage form of the invention, upon a 14-day oral administration in healthy adult subjects as described herein, provides a mean serum Tₘₐₓ of about 1.25 h on post-dosing day 14. In some embodiments, a 50 mg grapiprant unit dosage form of the invention, upon a 14-day oral administration in healthy adult subjects as described herein, provides a mean serum t_{1/2} of about 6.47 h on post-dosing day 14. In some embodiments, a 50 mg grapiprant unit dosage form of the invention, upon a 14-day oral administration in healthy adult subjects as described herein, provides a mean urine AE of about 10.6 mg on post-dosing day 14. In some embodiments, a 50 mg grapiprant unit dosage form of the invention, upon a 14-day oral administration in healthy adult subjects as described herein, provides a mean urine AE/Dose of about 21.2% on post-dosing day 14. In some embodiments, a 50 mg grapiprant unit dosage form of the invention, upon a 14-day oral administration in healthy adult subjects as described herein, provides a mean urine CL_{R} of about 56.3 mL/min on post-dosing day 14. In some embodiments, a 50 mg grapiprant unit dosage form of the invention, upon a 14-day oral administration in healthy adult subjects as described herein, provides a mean urine CL_{R}/Fu of about 6118 mL/min on post-dosing day 14.

In some embodiments, a 50 mg grapiprant unit dosage form of the invention, upon a 14-day oral administration in healthy adult subjects as described herein, provides one or more pharmacokinetics result on post-dosing day 1 selected from:

a serum Cₘₐₓ of about 578±about 381 ng/mL;

a serum AUC_{τ} of about 3150±about 1490 ng*h/mL; and

a serum Tₘₐₓ of about 0.50 to about 3.00 h.

In some embodiments, a 50 mg grapiprant unit dosage form of the invention, upon a 14-day oral administration in healthy adult subjects as described herein, provides a serum Cₘₐₓ of about 578±about 381 ng/mL on post-dosing day 1. In some embodiments, a 50 mg grapiprant unit dosage form of the invention, upon a 14-day oral administration in healthy adult subjects as described herein, provides a serum Cₘₐₓ of about 578±about 190 ng/mL on post-dosing day 1. In some embodiments, a 50 mg grapiprant unit dosage form of the invention, upon a 14-day oral administration in healthy adult subjects as described herein, provides a serum Cₘₐₓ of about 578±about 95 ng/mL on post-dosing day 1.

In some embodiments, a 50 mg grapiprant unit dosage form of the invention, upon a 14-day oral administration in healthy adult subjects as described herein, provides a serum AUC_{τ} of about 3150±about 1490 ng*h/mL on post-dosing day 1. In some embodiments, a 50 mg grapiprant unit dosage form of the invention, upon a 14-day oral administration in healthy adult subjects as described herein, provides a serum AUC_{τ} of about 3150±about 745 ng*h/mL on post-dosing day 1. In some embodiments, a 50 mg grapiprant unit dosage form of the invention, upon a 14-day oral administration in healthy adult subjects as described herein, provides a serum AUC_{τ} of about 3150±about 372 ng*h/mL on post-dosing day 1.

In some embodiments, a 50 mg grapiprant unit dosage form of the invention, upon a 14-day oral administration in healthy adult subjects as described herein, provides a serum Tₘₐₓ of about 0.5 to about 3.00 h on post-dosing day 1. In some embodiments, a 50 mg grapiprant unit dosage form of the invention, upon a 14-day oral administration in healthy adult subjects as described herein, provides a serum Tₘₐₓ of about 1.0 to about 2.25 hon post-dosing day 1. In some embodiments, a 50 mg grapiprant unit dosage form of the invention, upon a 14-day oral administration in healthy adult subjects as described herein, provides a serum Tₘₐₓ of about 1.25 to about 1.875 h on post-dosing day 1.

In some embodiments, a 50 mg grapiprant unit dosage form of the invention, upon a 14-day oral administration in healthy adult subjects as described herein, provides one or more pharmacokinetics result on post-dosing day 14 selected from:

a serum Cₘₐₓ of about 611±about 316 ng/mL;

a serum AUC_{τ} of about 3370±about 1420 ng*h/mL;

a serum Rac of about 1.13±about 0.153;

a serum Tₘₐₓ of about 0.50 to about 3.00 h;

a serum t_{1/2} of about 6.47±about 1.40 h;

a urine A_{E} of about 10.6±about 3.18 mg;

a urine A_{E}/Dose of about 21.2%±about 6.37%;

a urine CL_{R} of about 56.3±about 14.5 mL/min; and

a urine CL_{R}/Fu of about 6118±about 1580 mL/min.

In some embodiments, a 50 mg grapiprant unit dosage form of the invention, upon a 14-day oral administration in healthy adult subjects as described herein, provides a serum Cₘₐₓ of about 611±about 316 ng/mL on post-dosing day 14. In some embodiments, a 50 mg grapiprant unit dosage form of the invention, upon a 14-day oral administration in healthy adult subjects as described herein, provides a serum Cₘₐₓ of about 611±about 158 ng/mL on post-dosing day 14. In some embodiments, a 50 mg grapiprant unit dosage form of the invention, upon a 14-day oral administration in healthy adult subjects as described herein, provides a serum Cₘₐₓ of about 611±about 79 ng/mL on post-dosing day 14.

In some embodiments, a 50 mg grapiprant unit dosage form of the invention, upon a 14-day oral administration in healthy adult subjects as described herein, provides a serum AUC_{τ} of about 3370±about 1420 ng*h/mL on post-dosing day 14. In some embodiments, a 50 mg grapiprant unit dosage form of the invention, upon a 14-day oral administration in healthy adult subjects as described herein, provides a serum AUC_{τ} of about 3370±about 710 ng*h/mL on post-dosing day 14. In some embodiments, a 50 mg grapiprant unit dosage form of the invention, upon a 14-day oral administration in healthy adult subjects as described herein, provides a serum AUC_{τ} of about 3370±about 355 ng*h/mL on post-dosing day 14.

In some embodiments, a 50 mg grapiprant unit dosage form of the invention, upon a 14-day oral administration in healthy adult subjects as described herein, provides a serum Rac of about 1.13±about 0.153 on post-dosing day 14. In some embodiments, a 50 mg grapiprant unit dosage form of the invention, upon a 14-day oral administration in healthy adult subjects as described herein, provides a serum Rac of about 1.13±about 0.076 on post-dosing day 14. In some embodiments, a 50 mg grapiprant unit dosage form of the invention, upon a 14-day oral administration in healthy adult subjects as described herein, provides a serum Rac of about 1.13±about 0.038 on post-dosing day 14.

In some embodiments, a 50 mg grapiprant unit dosage form of the invention, upon a 14-day oral administration in healthy adult subjects as described herein, provides a serum Tₘₐₓ of about 0.50 to about 3.00 h on post-dosing day 14. In some embodiments, a 50 mg grapiprant unit dosage form of the invention, upon a 14-day oral administration in healthy adult subjects as described herein, provides a serum Tₘₐₓ of about 0.87 to about 2.12 h on post-dosing day 14. In some embodiments, a 50 mg grapiprant unit dosage form of the invention, upon a 14-day oral administration in healthy adult subjects as described herein, provides a serum Tₘₐₓ of about 1.06 to about 1.69 h on post-dosing day 14.

In some embodiments, a 50 mg grapiprant unit dosage form of the invention, upon a 14-day oral administration in healthy adult subjects as described herein, provides a serum t_{1/2} of about 6.47±about 1.40 h on post-dosing day 14. In some embodiments, a 50 mg grapiprant unit dosage form of the invention, upon a 14-day oral administration in healthy adult subjects as described herein, provides a serum t_{1/2} of about 6.47±about 0.70 h on post-dosing day 14. In some embodiments, a 50 mg grapiprant unit dosage form of the invention, upon a 14-day oral administration in healthy adult subjects as described herein, provides a serum t_{1/2} of about 6.47±about 0.35 h on post-dosing day 14.

In some embodiments, a 50 mg grapiprant unit dosage form of the invention, upon a 14-day oral administration in healthy adult subjects as described herein, provides a urine A_{E} of about 10.6±about 3.18 mg on post-dosing day 14. In some embodiments, a 50 mg grapiprant unit dosage form of the invention, upon a 14-day oral administration in healthy adult subjects as described herein, provides a urine A_{E} of about 10.6±about 1.59 mg on post-dosing day 14. In some embodiments, a 50 mg grapiprant unit dosage form of the invention, upon a 14-day oral administration in healthy adult subjects as described herein, provides a urine A_{E} of about 10.6±about 0.79 mg on post-dosing day 14.

In some embodiments, a 50 mg grapiprant unit dosage form of the invention, upon a 14-day oral administration in healthy adult subjects as described herein, provides a urine A_{E}/Dose of about 21.2%±about 6.37% on post-dosing day 14. In some embodiments, a 50 mg grapiprant unit dosage form of the invention, upon a 14-day oral administration in healthy adult subjects as described herein, provides a urine A_{E}/Dose of about 21.2%±about 3.18% on post-dosing day 14. In some embodiments, a 50 mg grapiprant unit dosage form of the invention, upon a 14-day oral administration in healthy adult subjects as described herein, provides a urine A_{E}/Dose of 21.2%±about 1.59% on post-dosing day 14.

In some embodiments, a 50 mg grapiprant unit dosage form of the invention, upon a 14-day oral administration in healthy adult subjects as described herein, provides a urine CL_{R} of about 56.3±about 14.5 mL/min on post-dosing day 14. In some embodiments, a 50 mg grapiprant unit dosage form of the invention, upon a 14-day oral administration in healthy adult subjects as described herein, provides a urine CL_{R} of about 56.3±about 7.25 mL/min on post-dosing day 14. In some embodiments, a 50 mg grapiprant unit dosage form of the invention, upon a 14-day oral administration in healthy adult subjects as described herein, provides a urine CL_{R} of about 56.3±about 3.62 mL/min on post-dosing day 14.

In some embodiments, a 50 mg grapiprant unit dosage form of the invention, upon a 14-day oral administration in healthy adult subjects as described herein, provides a urine CL_{R}/Fu of about 6118±about 1580 mL/min on post-dosing day 14. In some embodiments, a 50 mg grapiprant unit dosage form of the invention, upon a 14-day oral administration in healthy adult subjects as described herein, provides a urine CL_{R}/Fu of about 6118±about 790 mL/min on post-dosing day 14. In some embodiments, a 50 mg grapiprant unit dosage form of the invention, upon a 14-day oral administration in healthy adult subjects as described herein, provides a urine CL_{R}/Fu of about 6118±about 395 mL/min on post-dosing day 14.

In some embodiments, a 50 mg grapiprant unit dosage form of the invention, upon a 14-day oral administration in healthy adult subjects as described herein, provides one or more bone marker result on post-dosing days 7 and 14 selected from:

a LS Mean Difference of about −8.62% in bone-specific alkaline phosphatase on post-dosing day 7;

a LS Mean Difference of about 7.16% in bone-specific alkaline phosphatase on post-dosing day 14;

a LS Mean change of about −37.90% in blood osteocalcin on post-dosing day 7;

a LS Mean change of about −13.58% in blood osteocalcin on post-dosing day 14;

a LS Mean change of about 14.77% in N-telopeptide of type I collagen on post-dosing day 7; and

a LS Mean change of about −23.50% in N-telopeptide of type I collagen on post-dosing day 14.

In some embodiments, a 50 mg grapiprant unit dosage form of the invention, upon a 14-day oral administration in healthy adult subjects as described herein, provides a LS Mean Difference of about −8.62% in bone-specific alkaline phosphatase on post-dosing day 7. In some embodiments, a 50 mg grapiprant unit dosage form of the invention, upon a 14-day oral administration in healthy adult subjects as described herein, provides a LS Mean Difference of about 7.16% in bone-specific alkaline phosphatase on post-dosing day 14. In some embodiments, a 50 mg grapiprant unit dosage form of the invention, upon a 14-day oral administration in healthy adult subjects as described herein, provides a LS Mean change of about −37.90% in blood osteocalcin on post-dosing day 7. In some embodiments, a 50 mg grapiprant unit dosage form of the invention, upon a 14-day oral administration in healthy adult subjects as described herein, provides a LS Mean change of about −13.58% in blood osteocalcin on post-dosing day 14. In some embodiments, a 50 mg grapiprant unit dosage form of the invention, upon a 14-day oral administration in healthy adult subjects as described herein, provides a LS Mean change of about 14.77% in N-telopeptide of type I collagen on post-dosing day 7. In some embodiments, a 50 mg grapiprant unit dosage form of the invention, upon a 14-day oral administration in healthy adult subjects as described herein, provides a LS Mean change of about −23.50% in N-telopeptide of type I collagen on post-dosing day 14.

In some embodiments, a 50 mg grapiprant unit dosage form of the invention, upon a 14-day oral administration in healthy adult subjects as described herein, provides one or more bone marker result on post-dosing days 7 and 14 selected from:

a LS Mean Difference of about −22.00% to about 4.76% in bone-specific alkaline phosphatase on post-dosing day 7;

a LS Mean Difference of about −6.65% to about 20.98% in bone-specific alkaline phosphatase on post-dosing day 14;

a LS Mean change of about −70.12% to about −5.68% in blood osteocalcin on post-dosing day 7;

a LS Mean change of about −48.06% to about 20.89% in blood osteocalcin on post-dosing day 14;

a LS Mean change of about −23.15% to about 52.70% in N-telopeptide of type I collagen on post-dosing day 7; and

a LS Mean change of about −64.73% to about 17.74% in N-telopeptide of type I collagen on post-dosing day 14.

In some embodiments, a 50 mg grapiprant unit dosage form of the invention, upon a 14-day oral administration in healthy adult subjects as described herein, provides a LS Mean Difference of about −22.00% to about 4.76% in bone-specific alkaline phosphatase on post-dosing day 7. In some embodiments, a 50 mg grapiprant unit dosage form of the invention, upon a 14-day oral administration in healthy adult subjects as described herein, provides a LS Mean Difference of about −6.65% to about 20.98% in bone-specific alkaline phosphatase on post-dosing day 14. In some embodiments, a 50 mg grapiprant unit dosage form of the invention, upon a 14-day oral administration in healthy adult subjects as described herein, provides a LS Mean change of about −70.12% to about −5.68% in blood osteocalcin on post-dosing day 7. In some embodiments, a 50 mg grapiprant unit dosage form of the invention, upon a 14-day oral administration in healthy adult subjects as described herein, provides a LS Mean change of about −48.06% to about 20.89% in blood osteocalcin on post-dosing day 14. In some embodiments, a 50 mg grapiprant unit dosage form of the invention, upon a 14-day oral administration in healthy adult subjects as described herein, provides a LS Mean change of about −23.15% to about 52.70% in N-telopeptide of type I collagen on post-dosing day 7. In some embodiments, a 50 mg grapiprant unit dosage form of the invention, upon a 14-day oral administration in healthy adult subjects as described herein, provides a LS Mean change of about −64.73% to about 17.74% in N-telopeptide of type I collagen on post-dosing day 14.

In some embodiments, the present invention provides a unit dosage form of a pharmaceutical composition comprising about 150 mg of grapiprant, or a pharmaceutically acceptable salt thereof, and one or more pharmaceutically acceptable excipient or carrier (referred to herein as “a 150 mg grapiprant unit dosage form”)

In some embodiments, a 150 mg grapiprant unit dosage form of the invention, upon a 14-day oral administration in healthy adult subjects as described herein, provides one or more pharmacokinetics result on post-dosing day 1 selected from:

a mean serum Cₘₐₓ of about 2150 ng/mL;

a mean serum AUC_{τ} of about 8970 ng*h/mL; and

a mean serum Tₘₐₓ of about 1.5 h.

In some embodiments, a 150 mg grapiprant unit dosage form of the invention, upon a 14-day oral administration in healthy adult subjects as described herein, provides a mean serum Cₘₐₓ of about 2150 ng/mL on post-dosing day 1. In some embodiments, a 150 mg grapiprant unit dosage form of the invention, upon a 14-day oral administration in healthy adult subjects as described herein, provides a mean serum AUC_{τ} of about 8970 ng*h/mL on post-dosing day 1. In some embodiments, a 150 mg grapiprant unit dosage form of the invention, upon a 14-day oral administration in healthy adult subjects as described herein, provides a mean serum Tₘₐₓ of about 1.5 h on post-dosing day 1.

In some embodiments, a 150 mg grapiprant unit dosage form of the invention, upon a 14-day oral administration in healthy adult subjects as described herein, provides one or more pharmacokinetics result on post-dosing day 14 selected from:

a mean serum Cₘₐₓ of about 2670 ng/mL;

a mean serum AUC_{τ} of about 10300 ng*h/mL;

a mean serum Rac of about 1.20;

a mean serum Tₘₐₓ of about 1.50 h;

a mean serum t_{1/2} of about 9.71 h;

a mean urine A_{E} of about 33.3 mg;

a mean urine A_{E}/Dose of about 22.2%;

a mean urine CL_{R} of about 55.2 mL/min; and

a mean urine CL_{R}/Fu of about 6004 mL/min.

In some embodiments, a 150 mg grapiprant unit dosage form of the invention, upon a 14-day oral administration in healthy adult subjects as described herein, provides a mean serum Cₘₐₓ of about 2670 ng/mL on post-dosing day 14. In some embodiments, a 150 mg grapiprant unit dosage form of the invention, upon a 14-day oral administration in healthy adult subjects as described herein, provides a mean serum AUC_{τ} of about 10300 ng*h/mL on post-dosing day 14. In some embodiments, a 150 mg grapiprant unit dosage form of the invention, upon a 14-day oral administration in healthy adult subjects as described herein, provides a mean serum Rac of about 1.20 on post-dosing day 14. In some embodiments, a 150 mg grapiprant unit dosage form of the invention, upon a 14-day oral administration in healthy adult subjects as described herein, provides a mean serum Tₘₐₓ of about 1.50 h on post-dosing day 14. In some embodiments, a 150 mg grapiprant unit dosage form of the invention, upon a 14-day oral administration in healthy adult subjects as described herein, provides a mean serum t_{1/2} of about 9.71 h on post-dosing day 14. In some embodiments, a 150 mg grapiprant unit dosage form of the invention, upon a 14-day oral administration in healthy adult subjects as described herein, provides a mean urine A_{E} of about 33.3 mg on post-dosing day 14. In some embodiments, a 150 mg grapiprant unit dosage form of the invention, upon a 14-day oral administration in healthy adult subjects as described herein, provides a mean urine A_{E}/Dose of about 22.2% on post-dosing day 14. In some embodiments, a 150 mg grapiprant unit dosage form of the invention, upon a 14-day oral administration in healthy adult subjects as described herein, provides a mean urine CL_{R} of about 55.2 mL/min on post-dosing day 14. In some embodiments, a 150 mg grapiprant unit dosage form of the invention, upon a 14-day oral administration in healthy adult subjects as described herein, provides a mean urine CL_{R}/Fu of about 6004 mL/min on post-dosing day 14.

In some embodiments, a 150 mg grapiprant unit dosage form of the invention, upon a 14-day oral administration in healthy adult subjects as described herein, provides one or more pharmacokinetics result on post-dosing day 1 selected from:

a serum Cₘₐₓ of about 2150±about 1390 ng/mL;

a serum AUC_{τ} of about 8970±about 3880 ng*h/mL; and

a serum Tₘₐₓ of about 1.00 to about 3.00 h.

In some embodiments, a 150 mg grapiprant unit dosage form of the invention, upon a 14-day oral administration in healthy adult subjects as described herein, provides a serum Cₘₐₓ of about 2150±about 1390 ng/mL on post-dosing day 1. In some embodiments, a 150 mg grapiprant unit dosage form of the invention, upon a 14-day oral administration in healthy adult subjects as described herein, provides a serum Cₘₐₓ of about 2150±about 695 ng/mL on post-dosing day 1. In some embodiments, a 150 mg grapiprant unit dosage form of the invention, upon a 14-day oral administration in healthy adult subjects as described herein, provides a serum Cₘₐₓ of about 2150±about 347 ng/mL on post-dosing day 1.

In some embodiments, a 150 mg grapiprant unit dosage form of the invention, upon a 14-day oral administration in healthy adult subjects as described herein, provides a serum AUC_{τ} of about 8970±about 3880 ng*h/mL on post-dosing day 1. In some embodiments, a 150 mg grapiprant unit dosage form of the invention, upon a 14-day oral administration in healthy adult subjects as described herein, provides a serum AUC_{τ} of about 8970±about 1940 ng*h/mL on post-dosing day 1. In some embodiments, a 150 mg grapiprant unit dosage form of the invention, upon a 14-day oral administration in healthy adult subjects as described herein, provides a serum AUC_{τ} of about 8970±about 970 ng*h/mL on post-dosing day 1.

In some embodiments, a 150 mg grapiprant unit dosage form of the invention, upon a 14-day oral administration in healthy adult subjects as described herein, provides a serum Tₘₐₓ of about 1.00 to about 3.00 h on post-dosing day 1. In some embodiments, a 150 mg grapiprant unit dosage form of the invention, upon a 14-day oral administration in healthy adult subjects as described herein, provides a serum Tₘₐₓ of about 1.25 to about 2.25 h on post-dosing day 1. In some embodiments, a 150 mg grapiprant unit dosage form of the invention, upon a 14-day oral administration in healthy adult subjects as described herein, provides a serum Tₘₐₓ of about 1.375 to about 1.875 h on post-dosing day 1.

In some embodiments, a 150 mg grapiprant unit dosage form of the invention, upon a 14-day oral administration in healthy adult subjects as described herein, provides one or more pharmacokinetics result on post-dosing day 14 selected from:

a serum Cₘₐₓ of about 2670±about 1160 ng/mL;

a serum AUC_{τ} of about 10300±about 3350 ng*h/mL;

a serum Rac of about 1.20±about 0.165;

a serum Tₘₐₓ of about 0.50 to about 4.00 h;

a serum t_{1/2} of about 9.71±about 1.18 h;

a urine A_{E} of about 33.3±about 11.2 mg;

a urine A_{E}/Dose of about 22.2%±about 7.48%;

a urine CL_{R} of about 55.2±about 12.1 mL/min; and

a urine CL_{R}/Fu of about 6004±about 1313 mL/min.

In some embodiments, a 150 mg grapiprant unit dosage form of the invention, upon a 14-day oral administration in healthy adult subjects as described herein, provides a serum Cₘₐₓ of about 2670±about 1160 ng/mL on post-dosing day 14. In some embodiments, a 150 mg grapiprant unit dosage form of the invention, upon a 14-day oral administration in healthy adult subjects as described herein, provides a serum Cₘₐₓ of about 2670±about 580 ng/mL on post-dosing day 14. In some embodiments, a 150 mg grapiprant unit dosage form of the invention, upon a 14-day oral administration in healthy adult subjects as described herein, provides a serum Cₘₐₓ of about 2670±about 290 ng/mL on post-dosing day 14.

In some embodiments, a 150 mg grapiprant unit dosage form of the invention, upon a 14-day oral administration in healthy adult subjects as described herein, provides a serum AUC_{τ} of about 10300±about 3350 ng*h/mL on post-dosing day 14. In some embodiments, a 150 mg grapiprant unit dosage form of the invention, upon a 14-day oral administration in healthy adult subjects as described herein, provides a serum AUC_{τ} of about 10300±about 1675 ng*h/mL on post-dosing day 14. In some embodiments, a 150 mg grapiprant unit dosage form of the invention, upon a 14-day oral administration in healthy adult subjects as described herein, provides a serum AUC_{τ} of about 10300±about 837 ng*h/mL on post-dosing day 14.

In some embodiments, a 150 mg grapiprant unit dosage form of the invention, upon a 14-day oral administration in healthy adult subjects as described herein, provides a serum Rac of about 1.20±about 0.165 on post-dosing day 14. In some embodiments, a 150 mg grapiprant unit dosage form of the invention, upon a 14-day oral administration in healthy adult subjects as described herein, provides a serum Rac of about 1.20±about 0.0825 on post-dosing day 14. In some embodiments, a 150 mg grapiprant unit dosage form of the invention, upon a 14-day oral administration in healthy adult subjects as described herein, provides a serum Rac of about 1.20±about 0.041 on post-dosing day 14.

In some embodiments, a 150 mg grapiprant unit dosage form of the invention, upon a 14-day oral administration in healthy adult subjects as described herein, provides a serum Tₘₐₓ of about 0.50 to about 4.00 h on post-dosing day 14. In some embodiments, a 150 mg grapiprant unit dosage form of the invention, upon a 14-day oral administration in healthy adult subjects as described herein, provides a serum Tₘₐₓ of about 1.0 to about 2.75 h on post-dosing day 14. In some embodiments, a 150 mg grapiprant unit dosage form of the invention, upon a 14-day oral administration in healthy adult subjects as described herein, provides a serum Tₘₐₓ of about 1.25 to about 2.125 h on post-dosing day 14.

In some embodiments, a 150 mg grapiprant unit dosage form of the invention, upon a 14-day oral administration in healthy adult subjects as described herein, provides a serum t_{1/2} of about 9.71±about 1.18 hon post-dosing day 14. In some embodiments, a 150 mg grapiprant unit dosage form of the invention, upon a 14-day oral administration in healthy adult subjects as described herein, provides a serum t_{1/2} of about 9.71±about 0.59 h on post-dosing day 14. In some embodiments, a 150 mg grapiprant unit dosage form of the invention, upon a 14-day oral administration in healthy adult subjects as described herein, provides a serum t_{1/2} of about 9.71±about 0.29 h on post-dosing day 14.

In some embodiments, a 150 mg grapiprant unit dosage form of the invention, upon a 14-day oral administration in healthy adult subjects as described herein, provides a urine A_{E} of about 33.3±about 11.2 mg on post-dosing day 14. In some embodiments, a 150 mg grapiprant unit dosage form of the invention, upon a 14-day oral administration in healthy adult subjects as described herein, provides a urine A_{E} of about 33.3±about 5.6 mg on post-dosing day 14. In some embodiments, a 150 mg grapiprant unit dosage form of the invention, upon a 14-day oral administration in healthy adult subjects as described herein, provides a urine A_{E} of about 33.3±about 2.8 mg on post-dosing day 14.

In some embodiments, a 150 mg grapiprant unit dosage form of the invention, upon a 14-day oral administration in healthy adult subjects as described herein, provides a urine A_{E}/Dose of about 22.2%±about 7.48% on post-dosing day 14. In some embodiments, a 150 mg grapiprant unit dosage form of the invention, upon a 14-day oral administration in healthy adult subjects as described herein, provides a urine A_{E}/Dose of about 22.2%±about 3.74% on post-dosing day 14. In some embodiments, a 150 mg grapiprant unit dosage form of the invention, upon a 14-day oral administration in healthy adult subjects as described herein, provides a urine A_{E}/Dose of about 22.2%±about 1.87% on post-dosing day 14.

In some embodiments, a 150 mg grapiprant unit dosage form of the invention, upon a 14-day oral administration in healthy adult subjects as described herein, provides a urine CL_{R} of about 55.2±about 12.1 mL/min on post-dosing day 14. In some embodiments, a 150 mg grapiprant unit dosage form of the invention, upon a 14-day oral administration in healthy adult subjects as described herein, provides a urine CL_{R} of about 55.2±about 6.0 mL/min on post-dosing day 14. In some embodiments, a 150 mg grapiprant unit dosage form of the invention, upon a 14-day oral administration in healthy adult subjects as described herein, provides a urine CL_{R} of about 55.2±about 3.1 mL/min on post-dosing day 14.

In some embodiments, a 150 mg grapiprant unit dosage form of the invention, upon a 14-day oral administration in healthy adult subjects as described herein, provides a urine CL_{R}/Fu of about 6004±about 1313 mL/min on post-dosing day 14. In some embodiments, a 150 mg grapiprant unit dosage form of the invention, upon a 14-day oral administration in healthy adult subjects as described herein, provides a urine CL_{R}/Fu of about 6004±about 656 mL/min on post-dosing day 14. In some embodiments, a 150 mg grapiprant unit dosage form of the invention, upon a 14-day oral administration in healthy adult subjects as described herein, provides a urine CL_{R}/Fu of about 6004±about 329 mL/min on post-dosing day 14.

In some embodiments, a 150 mg grapiprant unit dosage form of the invention, upon a 14-day oral administration in healthy adult subjects as described herein, provides one or more bone marker result on post-dosing days 7 and 14 selected from:

a LS Mean Difference of about 4.76% in bone-specific alkaline phosphatase on post-dosing day 7;

a LS Mean Difference of about 1.23% in bone-specific alkaline phosphatase on post-dosing day 14;

a LS Mean change of about −31.02% in blood osteocalcin on post-dosing day 7;

a LS Mean change of about −10.29% in blood osteocalcin on post-dosing day 14;

a LS Mean change of about −9.78% in N-telopeptide of type I collagen on post-dosing day 7; and

a LS Mean change of about −38.72% in N-telopeptide of type I collagen on post-dosing day 14.

In some embodiments, a 150 mg grapiprant unit dosage form of the invention, upon a 14-day oral administration in healthy adult subjects as described herein, provides a LS Mean Difference of about 4.76% in bone-specific alkaline phosphatase on post-dosing day 7. In some embodiments, a 150 mg grapiprant unit dosage form of the invention, upon a 14-day oral administration in healthy adult subjects as described herein, provides a LS Mean Difference of about 1.23% in bone-specific alkaline phosphatase on post-dosing day 14. In some embodiments, a 150 mg grapiprant unit dosage form of the invention, upon a 14-day oral administration in healthy adult subjects as described herein, provides a LS Mean change of about −31.02% in blood osteocalcin on post-dosing day 7. In some embodiments, a 150 mg grapiprant unit dosage form of the invention, upon a 14-day oral administration in healthy adult subjects as described herein, provides a LS Mean change of about −10.29% in blood osteocalcin on post-dosing day 14. In some embodiments, a 150 mg grapiprant unit dosage form of the invention, upon a 14-day oral administration in healthy adult subjects as described herein, provides a LS Mean change of about −9.78% in N-telopeptide of type I collagen on post-dosing day 7. In some embodiments, a 150 mg grapiprant unit dosage form of the invention, upon a 14-day oral administration in healthy adult subjects as described herein, provides a LS Mean change of about −38.72% in N-telopeptide of type I collagen on post-dosing day 14.

In some embodiments, a 150 mg grapiprant unit dosage form of the invention, upon a 14-day oral administration in healthy adult subjects as described herein, provides one or more bone markers on post-dosing days 7 and 14 selected from:

a LS Mean Difference of about −9.66% to about 19.19% in bone-specific alkaline phosphatase on post-dosing day 7;

a LS Mean Difference of about −13.35% to about 15.80% in bone-specific alkaline phosphatase on post-dosing day 14;

a LS Mean change of about −63.31% to about 1.26% in blood osteocalcin on post-dosing day 7;

a LS Mean change of about −43.42% to about 22.84% in blood osteocalcin on post-dosing day 14;

a LS Mean change of about −47.36% to about 27.79% in N-telopeptide of type I collagen on post-dosing day 7; and

a LS Mean change of about −77.39% to about −0.04% in N-telopeptide of type I collagen on post-dosing day 14.

In some embodiments, a 150 mg grapiprant unit dosage form of the invention, upon a 14-day oral administration in healthy adult subjects as described herein, provides a LS Mean Difference of about −9.66% to about 19.19% in bone-specific alkaline phosphatase on post-dosing day 7. In some embodiments, a 150 mg grapiprant unit dosage form of the invention, upon a 14-day oral administration in healthy adult subjects as described herein, provides a LS Mean Difference of about −13.35% to about 15.80% in bone-specific alkaline phosphatase on post-dosing day 14. In some embodiments, a 150 mg grapiprant unit dosage form of the invention, upon a 14-day oral administration in healthy adult subjects as described herein, provides a LS Mean change of about −63.31% to about 1.26% in blood osteocalcin on post-dosing day 7. In some embodiments, a 150 mg grapiprant unit dosage form of the invention, upon a 14-day oral administration in healthy adult subjects as described herein, provides a LS Mean change of about −43.42% to about 22.84% in blood osteocalcin on post-dosing day 14. In some embodiments, a 150 mg grapiprant unit dosage form of the invention, upon a 14-day oral administration in healthy adult subjects as described herein, provides a LS Mean change of about −47.36% to about 27.79% in N-telopeptide of type I collagen on post-dosing day 7. In some embodiments, a 150 mg grapiprant unit dosage form of the invention, upon a 14-day oral administration in healthy adult subjects as described herein, provides a LS Mean change of about −77.39% to about −0.04% in N-telopeptide of type I collagen on post-dosing day 14.

In some embodiments, a unit dosage form of a pharmaceutical composition comprises about 250 mg of grapiprant, or a pharmaceutically acceptable salt thereof, and one or more pharmaceutically acceptable excipient or carrier (referred to herein as “a 250 mg grapiprant unit dosage form”).

In some embodiments, a 250 mg grapiprant unit dosage form of the invention, upon a 17-day oral administration in elderly subjects with minor renal impairment as described herein, provides one or more pharmacokinetics result on post-dosing day 1 selected from:

a mean serum Cₘₐₓ of about 10300 ng/mL;

a mean serum AUC_{τ} of about 30100 ng*h/mL;

a mean serum Tₘₐₓ of about 1.00 h; and

a mean serum T1/2 of about 8.63 h.

In some embodiments, a 250 mg grapiprant unit dosage form of the invention, upon a 17-day oral administration in elderly subjects with minor renal impairment as described herein, provides a mean serum Cₘₐₓ of about 10300 ng/mL on post-dosing day 1. In some embodiments, a 250 mg grapiprant unit dosage form of the invention, upon a 17-day oral administration in elderly subjects with minor renal impairment as described herein, provides a mean serum AUC_{τ} of about 30100 ng*h/mL on post-dosing day 1. In some embodiments, a 250 mg grapiprant unit dosage form of the invention, upon a 17-day oral administration in elderly subjects with minor renal impairment as described herein, provides a mean serum Tₘₐₓ of about 1.00 h on post-dosing day 1. In some embodiments, a 250 mg grapiprant unit dosage form of the invention, upon a 17-day oral administration in elderly subjects with minor renal impairment as described herein, provides a mean serum T1/2 of about 8.63 h on post-dosing day 1.

In some embodiments, a 250 mg grapiprant unit dosage form of the invention, upon a 17-day oral administration in elderly subjects with minor renal impairment as described herein, provides one or more pharmacokinetics result on post-dosing day 17 selected from:

a mean serum Cₘₐₓ of about 12300 ng/mL;

a mean serum AUC_{τ} of about 40200 ng*h/mL;

a mean serum Rac of about 1.54;

a mean serum Tₘₐₓ of about 1.00 h;

a mean urine A_{E} of about 57.9 mg;

a mean urine A_{E}/Dose of about 23.2%;

a mean urine CL_{R} of about 25.0 mL/min; and

a mean urine CL_{R}/Fu of about 2719 mL/min.

In some embodiments, a 250 mg grapiprant unit dosage form of the invention, upon a 17-day oral administration in elderly subjects with minor renal impairment as described herein, provides a mean serum Cₘₐₓ of about 12300 ng/mL on post-dosing day 17. In some embodiments, a 250 mg grapiprant unit dosage form of the invention, upon a 17-day oral administration in elderly subjects with minor renal impairment as described herein, provides a mean serum AUC_{τ} of about 40200 ng*h/mL on post-dosing day 17. In some embodiments, a 250 mg grapiprant unit dosage form of the invention, upon a 17-day oral administration in elderly subjects with minor renal impairment as described herein, provides a mean serum Rac of about 1.54 on post-dosing day 17. In some embodiments, a 250 mg grapiprant unit dosage form of the invention, upon a 17-day oral administration in elderly subjects with minor renal impairment as described herein, provides a mean serum Tₘₐₓ of about 1.00 h on post-dosing day 17. In some embodiments, a 250 mg grapiprant unit dosage form of the invention, upon a 17-day oral administration in elderly subjects with minor renal impairment as described herein, provides a mean urine A_{E} of about 57.9 mg on post-dosing day 17. In some embodiments, a 250 mg grapiprant unit dosage form of the invention, upon a 17-day oral administration in elderly subjects with minor renal impairment as described herein, provides a mean urine A_{E}/Dose of about 23.2% on post-dosing day 17. In some embodiments, a 250 mg grapiprant unit dosage form of the invention, upon a 17-day oral administration in elderly subjects with minor renal impairment as described herein, provides a mean urine CL_{R} of about 25.0 mL/min on post-dosing day 17. In some embodiments, a 250 mg grapiprant unit dosage form of the invention, upon a 17-day oral administration in elderly subjects with minor renal impairment as described herein, provides a mean urine CL_{R}/Fu of about 2719 mL/min on post-dosing day 17.

In some embodiments, a 250 mg grapiprant unit dosage form of the invention, upon a 17-day oral administration in elderly subjects with minor renal impairment as described herein, provides one or more pharmacokinetics result on post-dosing day 1 selected from:

a serum Cₘₐₓ of about 10300±about 5710 ng/mL;

an AUC_{τ} of about 30100±about 12800 ng*h/mL;

a Tₘₐₓ of about 0.50 to about 1.50 h; and

a serum T1/2 of about 8.63±about 2.22 h.

In some embodiments, a 250 mg grapiprant unit dosage form of the invention, upon a 17-day oral administration in elderly subjects with minor renal impairment as described herein, provides a serum Cₘₐₓ of about 10300±about 5710 ng/mL on post-dosing day 1. In some embodiments, a 250 mg grapiprant unit dosage form of the invention, upon a 17-day oral administration in elderly subjects with minor renal impairment as described herein, provides a serum Cₘₐₓ of about 10300±about 2855 ng/mL on post-dosing day 1. In some embodiments, a 250 mg grapiprant unit dosage form of the invention, upon a 17-day oral administration in elderly subjects with minor renal impairment as described herein, provides a serum Cₘₐₓ of about 10300±about 1427 ng/mL on post-dosing day 1.

In some embodiments, a 250 mg grapiprant unit dosage form of the invention, upon a 17-day oral administration in elderly subjects with minor renal impairment as described herein, provides an AUC_{τ} of about 30100±about 12800 ng*h/mL on post-dosing day 1. In some embodiments, a 250 mg grapiprant unit dosage form of the invention, upon a 17-day oral administration in elderly subjects with minor renal impairment as described herein, provides an AUC_{τ} of about 30100±about 6400 ng*h/mL on post-dosing day 1. In some embodiments, a 250 mg grapiprant unit dosage form of the invention, upon a 17-day oral administration in elderly subjects with minor renal impairment as described herein, provides an AUC_{τ} of about 30100±about 3200 ng*h/mL on post-dosing day 1.

In some embodiments, a 250 mg grapiprant unit dosage form of the invention, upon a 17-day oral administration in elderly subjects with minor renal impairment as described herein, provides a Tₘₐₓ of about 0.50 to about 1.50 hon post-dosing day 1. In some embodiments, a 250 mg grapiprant unit dosage form of the invention, upon a 17-day oral administration in elderly subjects with minor renal impairment as described herein, provides a Tₘₐₓ of about 0.75 to about 1.25 h on post-dosing day 1. In some embodiments, a 250 mg grapiprant unit dosage form of the invention, upon a 17-day oral administration in elderly subjects with minor renal impairment as described herein, provides a Tₘₐₓ of about 0.88 to about 1.12 h on post-dosing day 1.

In some embodiments, a 250 mg grapiprant unit dosage form of the invention, upon a 17-day oral administration in elderly subjects with minor renal impairment as described herein, provides a serum T_{1/2} of about 8.63±about 2.22 h on post-dosing day 1. In some embodiments, a 250 mg grapiprant unit dosage form of the invention, upon a 17-day oral administration in elderly subjects with minor renal impairment as described herein, provides a serum T_{1/2} of about 8.63±about 1.11 h on post-dosing day 1. In some embodiments, a 250 mg grapiprant unit dosage form of the invention, upon a 17-day oral administration in elderly subjects with minor renal impairment as described herein, provides a serum T1/2 of about 8.63±about 0.55 h on post-dosing day 1.

In some embodiments, a 250 mg grapiprant unit dosage form of the invention, upon a 17-day oral administration in elderly subjects with minor renal impairment as described herein, provides one or more pharmacokinetics result on post-dosing day 17 selected from:

a serum Cₘₐₓ of about 12300±about 4020 ng/mL;

a serum AUC_{τ} of about 40200±about 17300 ng*h/mL;

a serum R_{ac} of about 1.54±about 0.841;

a serum Tₘₐₓ of about 0.50 to about 1.50 h;

a urine A_{E} of about 57.9±about 18.7 mg;

a urine A_{E}/Dose of about 23.2%±about 7.48%;

a urine CL_{R} of about 25.0±about 4.69 mL/min; and

a urine CL_{R}/Fu of 2719±about 510 mL/min.

In some embodiments, a 250 mg grapiprant unit dosage form of the invention, upon a 17-day oral administration in elderly subjects with minor renal impairment as described herein, provides a serum Cₘₐₓ of about 12300±about 4020 ng/mL on post-dosing day 17. In some embodiments, a 250 mg grapiprant unit dosage form of the invention, upon a 17-day oral administration in elderly subjects with minor renal impairment as described herein, provides a serum Cₘₐₓ of about 12300±about 2010 ng/mL on post-dosing day 17. In some embodiments, a 250 mg grapiprant unit dosage form of the invention, upon a 17-day oral administration in elderly subjects with minor renal impairment as described herein, provides a serum Cₘₐₓ of about 12300±about 1005 ng/mL on post-dosing day 17.

In some embodiments, a 250 mg grapiprant unit dosage form of the invention, upon a 17-day oral administration in elderly subjects with minor renal impairment as described herein, provides a serum AUC_{τ} of about 40200±about 17300 ng*h/mL on post-dosing day 17. In some embodiments, a 250 mg grapiprant unit dosage form of the invention, upon a 17-day oral administration in elderly subjects with minor renal impairment as described herein, provides a serum AUC_{τ} of about 40200±about 8650 ng*h/mL on post-dosing day 17. In some embodiments, a 250 mg grapiprant unit dosage form of the invention, upon a 17-day oral administration in elderly subjects with minor renal impairment as described herein, provides a serum AUC_{τ} of about 40200±about 4325 ng*h/mL on post-dosing day 17.

In some embodiments, a 250 mg grapiprant unit dosage form of the invention, upon a 17-day oral administration in elderly subjects with minor renal impairment as described herein, provides a serum R_{ac} of about 1.54±about 0.841 on post-dosing day 17. In some embodiments, a 250 mg grapiprant unit dosage form of the invention, upon a 17-day oral administration in elderly subjects with minor renal impairment as described herein, provides a serum Rac of about 1.54±about 0.42 on post-dosing day 17. In some embodiments, a 250 mg grapiprant unit dosage form of the invention, upon a 17-day oral administration in elderly subjects with minor renal impairment as described herein, provides a serum Rac of about 1.54±about 0.21 on post-dosing day 17.

In some embodiments, a 250 mg grapiprant unit dosage form of the invention, upon a 17-day oral administration in elderly subjects with minor renal impairment as described herein, provides a serum Tₘₐₓ of about 0.50 to about 1.50 hon post-dosing day 17. In some embodiments, a 250 mg grapiprant unit dosage form of the invention, upon a 17-day oral administration in elderly subjects with minor renal impairment as described herein, provides a serum Tₘₐₓ of about 0.75 to about 1.25 h on post-dosing day 17. In some embodiments, a 250 mg grapiprant unit dosage form of the invention, upon a 17-day oral administration in elderly subjects with minor renal impairment as described herein, provides a serum Tₘₐₓ of about 0.875 to about 1.125 hon post-dosing day 17.

In some embodiments, a 250 mg grapiprant unit dosage form of the invention, upon a 17-day oral administration in elderly subjects with minor renal impairment as described herein, provides a urine A_{E} of about 57.9±about 18.7 mg on post-dosing day 17. In some embodiments, a 250 mg grapiprant unit dosage form of the invention, upon a 17-day oral administration in elderly subjects with minor renal impairment as described herein, provides a urine A_{E} of about 57.9±about 9.3 mg on post-dosing day 17. In some embodiments, a 250 mg grapiprant unit dosage form of the invention, upon a 17-day oral administration in elderly subjects with minor renal impairment as described herein, provides a urine A_{E} of about 57.9±about 4.7 mg on post-dosing day 17.

In some embodiments, a 250 mg grapiprant unit dosage form of the invention, upon a 17-day oral administration in elderly subjects with minor renal impairment as described herein, provides a urine A_{E}/Dose of about 23.2%±about 7.48% on post-dosing day 17. In some embodiments, a 250 mg grapiprant unit dosage form of the invention, upon a 17-day oral administration in elderly subjects with minor renal impairment as described herein, provides a urine A_{E}/Dose of about 23.2%±about 3.74% on post-dosing day 17. In some embodiments, a 250 mg grapiprant unit dosage form of the invention, upon a 17-day oral administration in elderly subjects with minor renal impairment as described herein, provides a urine A_{E}/Dose of about 23.2%±about 1.87% on post-dosing day 17.

In some embodiments, a 250 mg grapiprant unit dosage form of the invention, upon a 17-day oral administration in elderly subjects with minor renal impairment as described herein, provides a urine CL_{R} of about 25.0±about 4.69 mL/min on post-dosing day 17. In some embodiments, a 250 mg grapiprant unit dosage form of the invention, upon a 17-day oral administration in elderly subjects with minor renal impairment as described herein, provides a urine CL_{R} of about 25.0±about 2.34 mL/min on post-dosing day 17. In some embodiments, a 250 mg grapiprant unit dosage form of the invention, upon a 17-day oral administration in elderly subjects with minor renal impairment as described herein, provides a urine CL_{R} of about 25.0±about 1.17 mL/min on post-dosing day 17.

In some embodiments, a 250 mg grapiprant unit dosage form of the invention, upon a 17-day oral administration in elderly subjects with minor renal impairment as described herein, provides a urine CL_{R}/Fu of about 2719±about 510 mL/min on post-dosing day 17. In some embodiments, a 250 mg grapiprant unit dosage form of the invention, upon a 17-day oral administration in elderly subjects with minor renal impairment as described herein, provides a urine CL_{R}/Fu of about 2719±about 255 mL/min on post-dosing day 17. In some embodiments, a 250 mg grapiprant unit dosage form of the invention, upon a 17-day oral administration in elderly subjects with minor renal impairment as described herein, provides a urine CL_{R}/Fu of about 2719±about 127 mL/min on post-dosing day 17.

In some embodiments, a 250 mg grapiprant unit dosage form of the invention, upon a 17-day oral administration in elderly subjects with minor renal impairment as described herein, provides one or more bone marker result on post-dosing days 10 and 17 selected from:

a LS Mean Difference of about −1.75% in bone-specific alkaline phosphatase on post-dosing day 10;

a LS Mean Difference of about 3.88% in bone-specific alkaline phosphatase on post-dosing day 17;

a LS Mean change of about −5.91% in blood osteocalcin on post-dosing day 10;

a LS Mean change of about −3.96% in blood osteocalcin on post-dosing day 17;

a LS Mean change of about −9.62% in N-telopeptide of type I collagen on post-dosing day 10; and

a LS Mean change of about −16.76% in N-telopeptide of type I collagen on post-dosing day 17.

In some embodiments, a 250 mg grapiprant unit dosage form of the invention, upon a 17-day oral administration in elderly subjects with minor renal impairment as described herein, provides a LS Mean Difference of about −1.75% in bone-specific alkaline phosphatase on post-dosing day 10. In some embodiments, a 250 mg grapiprant unit dosage form of the invention, upon a 17-day oral administration in elderly subjects with minor renal impairment as described herein, provides a LS Mean Difference of about 3.88% in bone-specific alkaline phosphatase on post-dosing day 17. In some embodiments, a 250 mg grapiprant unit dosage form of the invention, upon a 17-day oral administration in elderly subjects with minor renal impairment as described herein, provides a LS Mean change of about −5.91% in blood osteocalcin on post-dosing day 10. In some embodiments, a 250 mg grapiprant unit dosage form of the invention, upon a 17-day oral administration in elderly subjects with minor renal impairment as described herein, provides a LS Mean change of about −3.96% in blood osteocalcin on post-dosing day 17. In some embodiments, a 250 mg grapiprant unit dosage form of the invention, upon a 17-day oral administration in elderly subjects with minor renal impairment as described herein, provides a LS Mean change of about −9.62% in N-telopeptide of type I collagen on post-dosing day 10. In some embodiments, a 250 mg grapiprant unit dosage form of the invention, upon a 17-day oral administration in elderly subjects with minor renal impairment as described herein, provides a LS Mean change of about −16.76% in N-telopeptide of type I collagen on post-dosing day 17.

In some embodiments, a 250 mg grapiprant unit dosage form of the invention, upon a 17-day oral administration in elderly subjects with minor renal impairment as described herein, provides one or more bone marker result on post-dosing days 10 and 17 selected from:

a LS Mean Difference of about −18.60% to about 15.11% in bone-specific alkaline phosphatase on post-dosing day 10;

a LS Mean Difference of about −12.98% to about 20.73% in bone-specific alkaline phosphatase on post-dosing day 17;

a LS Mean change of about −31.72% to about 19.90% in blood osteocalcin on post-dosing day 10;

a LS Mean change of about −29.77% to about 21.85% in blood osteocalcin on post-dosing day 17;

a LS Mean change of about −39.25% to about 20.01% in N-telopeptide of type I collagen on post-dosing day 10; and

a LS Mean change of about −46.39% to about 12.87% in N-telopeptide of type I collagen on post-dosing day 17.

In some embodiments, a 250 mg grapiprant unit dosage form of the invention, upon a 17-day oral administration in elderly subjects with minor renal impairment as described herein, provides a LS Mean Difference of about −18.60% to about 15.11% in bone-specific alkaline phosphatase on post-dosing day 10. In some embodiments, a 250 mg grapiprant unit dosage form of the invention, upon a 17-day oral administration in elderly subjects with minor renal impairment as described herein, provides a LS Mean Difference of about −12.98% to about 20.73% in bone-specific alkaline phosphatase on post-dosing day 17. In some embodiments, a 250 mg grapiprant unit dosage form of the invention, upon a 17-day oral administration in elderly subjects with minor renal impairment as described herein, provides a LS Mean change of about −31.72% to about 19.90% in blood osteocalcin on post-dosing day 10. In some embodiments, a 250 mg grapiprant unit dosage form of the invention, upon a 17-day oral administration in elderly subjects with minor renal impairment as described herein, provides a LS Mean change of about −29.77% to about 21.85% in blood osteocalcin on post-dosing day 17. In some embodiments, a 250 mg grapiprant unit dosage form of the invention, upon a 17-day oral administration in elderly subjects with minor renal impairment as described herein, provides a LS Mean change of about −39.25% to about 20.01% in N-telopeptide of type I collagen on post-dosing day 10. In some embodiments, a 250 mg grapiprant unit dosage form of the invention, upon a 17-day oral administration in elderly subjects with minor renal impairment as described herein, provides a LS Mean change of about −46.39% to about 12.87% in N-telopeptide of type I collagen on post-dosing day 17.

In some embodiments, a unit dosage form of a pharmaceutical composition comprises about 300 mg of grapiprant, or a pharmaceutically acceptable salt thereof, and one or more pharmaceutically acceptable excipient or carrier (referred to herein as “a 300 mg grapiprant unit dosage form.”).

In some embodiments, a 300 mg grapiprant unit dosage form of the invention, upon a 14-day oral administration in healthy adult subjects as described herein, provides one or more pharmacokinetics result on post-dosing day 1 selected from:

a mean serum Cₘₐₓ of about 8240 ng/mL;

a mean serum AUC_{τ} of about 24900 ng*h/mL; and

a mean serum Tₘₐₓ of about 1.5 h.

In some embodiments, a 300 mg grapiprant unit dosage form of the invention, upon a 14-day oral administration in healthy adult subjects as described herein, provides a mean serum Cₘₐₓ of about 8240 ng/mL on post-dosing day 1. In some embodiments, a 300 mg grapiprant unit dosage form of the invention, upon a 14-day oral administration in healthy adult subjects as described herein, provides a mean serum AUC_{τ} of about 24900 ng*h/mL on post-dosing day 1. In some embodiments, a 300 mg grapiprant unit dosage form of the invention, upon a 14-day oral administration in healthy adult subjects as described herein, provides a mean serum Tₘₐₓ of about 1.5 h on post-dosing day 1.

In some embodiments, a 300 mg grapiprant unit dosage form of the invention, upon a 14-day oral administration in healthy adult subjects as described herein, provides one or more pharmacokinetics result on post-dosing day 14 selected from:

a mean serum Cₘₐₓ of about 10400 ng/mL;

a mean serum AUC_{τ} of about 32300 ng*h/mL;

a mean serum R_{ac} of about 1.33;

a mean serum Tₘₐₓ of about 1.00 h;

a mean serum t_{1/2} of about 8.76 h;

a mean urine A_{E} of about 82.4 mg;

a mean urine A_{E}/Dose of about 27.5%;

a mean urine CL_{R} of about 44.5 mL/min; and

a mean urine CL_{R}/Fu of about 4838 mL/min.

In some embodiments, a 300 mg grapiprant unit dosage form of the invention, upon a 14-day oral administration in healthy adult subjects as described herein, provides a mean serum Cₘₐₓ of about 10400 ng/mL on post-dosing day 14. In some embodiments, a 300 mg grapiprant unit dosage form of the invention, upon a 14-day oral administration in healthy adult subjects as described herein, provides a mean serum AUC_{τ} of about 32300 ng*h/mL on post-dosing day 14. In some embodiments, a 300 mg grapiprant unit dosage form of the invention, upon a 14-day oral administration in healthy adult subjects as described herein, provides a mean serum Rac of about 1.33 on post-dosing day 14. In some embodiments, a 300 mg grapiprant unit dosage form of the invention, upon a 14-day oral administration in healthy adult subjects as described herein, provides a mean serum Tₘₐₓ of about 1.00 h on post-dosing day 14. In some embodiments, a 300 mg grapiprant unit dosage form of the invention, upon a 14-day oral administration in healthy adult subjects as described herein, provides a mean serum t_{1/2} of about 8.76 h on post-dosing day 14. In some embodiments, a 300 mg grapiprant unit dosage form of the invention, upon a 14-day oral administration in healthy adult subjects as described herein, provides a mean urine A_{E} of about 82.4 mg on post-dosing day 14. In some embodiments, a 300 mg grapiprant unit dosage form of the invention, upon a 14-day oral administration in healthy adult subjects as described herein, provides a mean urine A_{E}/Dose of about 27.5% on post-dosing day 14. In some embodiments, a 300 mg grapiprant unit dosage form of the invention, upon a 14-day oral administration in healthy adult subjects as described herein, provides a mean urine CL_{R} of about 44.5 mL/min on post-dosing day 14. In some embodiments, a 300 mg grapiprant unit dosage form of the invention, upon a 14-day oral administration in healthy adult subjects as described herein, provides a mean urine CL_{R}/Fu of about 4838 mL/min on post-dosing day 14.

In some embodiments, a 300 mg grapiprant unit dosage form of the invention, upon a 14-day oral administration in healthy adult subjects as described herein, provides one or more pharmacokinetics result on post-dosing day 1 selected from:

a serum Cₘₐₓ of about 8240±about 5390 ng/mL;

a serum AUC_{τ} of about 24900±about 10300 ng*h/mL; and

a serum Tₘₐₓ of about 0.50 to about 4.00 h.

In some embodiments, a 300 mg grapiprant unit dosage form of the invention, upon a 14-day oral administration in healthy adult subjects as described herein, provides a serum Cₘₐₓ of about 8240±about 5390 ng/mL on post-dosing day 1. In some embodiments, a 300 mg grapiprant unit dosage form of the invention, upon a 14-day oral administration in healthy adult subjects as described herein, provides a serum Cₘₐₓ of about 8240±about 2695 ng/mL on post-dosing day 1. In some embodiments, a 300 mg grapiprant unit dosage form of the invention, upon a 14-day oral administration in healthy adult subjects as described herein, provides a serum Cₘₐₓ of about 8240±about 1348 ng/mL on post-dosing day 1.

In some embodiments, a 300 mg grapiprant unit dosage form of the invention, upon a 14-day oral administration in healthy adult subjects as described herein, provides a serum AUC_{τ} of about 24900±about 10300 ng*h/mL on post-dosing day 1. In some embodiments, a 300 mg grapiprant unit dosage form of the invention, upon a 14-day oral administration in healthy adult subjects as described herein, provides a serum AUC_{τ} of about 24900±about 5150 ng*h/mL on post-dosing day 1. In some embodiments, a 300 mg grapiprant unit dosage form of the invention, upon a 14-day oral administration in healthy adult subjects as described herein, provides a serum AUC_{τ} of about 24900±about 2575 ng*h/mL on post-dosing day 1.

In some embodiments, a 300 mg grapiprant unit dosage form of the invention, upon a 14-day oral administration in healthy adult subjects as described herein, provides a serum Tₘₐₓ of about 0.50 to about 4.00 h on post-dosing day 1. In some embodiments, a 300 mg grapiprant unit dosage form of the invention, upon a 14-day oral administration in healthy adult subjects as described herein, provides a serum Tₘₐₓ of about 1.00 to about 2.75 h on post-dosing day 1. In some embodiments, a 300 mg grapiprant unit dosage form of the invention, upon a 14-day oral administration in healthy adult subjects as described herein, provides a serum Tₘₐₓ of about 1.25 to about 2.125 h on post-dosing day 1.

In some embodiments, a 300 mg grapiprant unit dosage form of the invention, upon a 14-day oral administration in healthy adult subjects as described herein, provides one or more pharmacokinetics result on post-dosing day 14 selected from:

a serum Cₘₐₓ of about 10400±about 5000 ng/mL;

a serum AUC_{τ} of about 32300±about 11900 ng*h/mL;

a serum Rac of about 1.33±about 0.334;

a serum Tₘₐₓ of about 0.50 to about 4.00 h;

a serum t_{1/2} of about 8.76±about 2.35 h;

a urine A_{E} of about 82.4±about 27.1 mg;

a urine A_{E}/Dose of about 27.5±about 9.05%;

a urine CL_{R} of about 44.5±about 10.0 mL/min; and

a urine CL_{R}/Fu of about 4838±about 1085 mL/min.

In some embodiments, a 300 mg grapiprant unit dosage form of the invention, upon a 14-day oral administration in healthy adult subjects as described herein, provides a serum Cₘₐₓ of about 10400±about 5000 ng/mL on post-dosing day 14. In some embodiments, a 300 mg grapiprant unit dosage form of the invention, upon a 14-day oral administration in healthy adult subjects as described herein, provides a serum Cₘₐₓ of about 10400±about 2500 ng/mL on post-dosing day 14. In some embodiments, a 300 mg grapiprant unit dosage form of the invention, upon a 14-day oral administration in healthy adult subjects as described herein, provides a serum Cₘₐₓ of about 10400±about 1250 ng/mL on post-dosing day 14.

In some embodiments, a 300 mg grapiprant unit dosage form of the invention, upon a 14-day oral administration in healthy adult subjects as described herein, provides a serum AUC_{τ} of about 32300±about 11900 ng*h/mL on post-dosing day 14. In some embodiments, a 300 mg grapiprant unit dosage form of the invention, upon a 14-day oral administration in healthy adult subjects as described herein, provides a serum AUC_{τ} of about 32300±about 5950 ng*h/mL on post-dosing day 14. In some embodiments, a 300 mg grapiprant unit dosage form of the invention, upon a 14-day oral administration in healthy adult subjects as described herein, provides a serum AUC_{τ} of about 32300±about 2975 ng*h/mL on post-dosing day 14.

In some embodiments, a 300 mg grapiprant unit dosage form of the invention, upon a 14-day oral administration in healthy adult subjects as described herein, provides a serum Rac of about 1.33±about 0.334 on post-dosing day 14. In some embodiments, a 300 mg grapiprant unit dosage form of the invention, upon a 14-day oral administration in healthy adult subjects as described herein, provides a serum Rac of about 1.33±about 0.167 on post-dosing day 14. In some embodiments, a 300 mg grapiprant unit dosage form of the invention, upon a 14-day oral administration in healthy adult subjects as described herein, provides a serum Rac of about 1.33±about 0.084 on post-dosing day 14.

In some embodiments, a 300 mg grapiprant unit dosage form of the invention, upon a 14-day oral administration in healthy adult subjects as described herein, provides a serum Tₘₐₓ of about 0.50 to about 4.00 h on post-dosing day 14. In some embodiments, a 300 mg grapiprant unit dosage form of the invention, upon a 14-day oral administration in healthy adult subjects as described herein, provides a serum Tₘₐₓ of about 1.00 to about 2.75 h on post-dosing day 14. In some embodiments, a 300 mg grapiprant unit dosage form of the invention, upon a 14-day oral administration in healthy adult subjects as described herein, provides a serum Tₘₐₓ of about 1.25 to about 2.125 h on post-dosing day 14.

In some embodiments, a 300 mg grapiprant unit dosage form of the invention, upon a 14-day oral administration in healthy adult subjects as described herein, provides a serum t_{1/2} of about 8.76±about 2.35 h on post-dosing day 14. In some embodiments, a 300 mg grapiprant unit dosage form of the invention, upon a 14-day oral administration in healthy adult subjects as described herein, provides a serum t_{1/2} of about 8.76±about 1.18 h on post-dosing day 14. In some embodiments, a 300 mg grapiprant unit dosage form of the invention, upon a 14-day oral administration in healthy adult subjects as described herein, provides a serum t_{1/2} of about 8.76±about 0.59 h on post-dosing day 14.

In some embodiments, a 300 mg grapiprant unit dosage form of the invention, upon a 14-day oral administration in healthy adult subjects as described herein, provides a urine A_{E} of about 82.4±about 27.1 mg on post-dosing day 14. In some embodiments, a 300 mg grapiprant unit dosage form of the invention, upon a 14-day oral administration in healthy adult subjects as described herein, provides a urine A_{E} of about 82.4±about 13.6 mg on post-dosing day 14. In some embodiments, a 300 mg grapiprant unit dosage form of the invention, upon a 14-day oral administration in healthy adult subjects as described herein, provides a urine A_{E} of about 82.4±about 6.8 mg on post-dosing day 14.

In some embodiments, a 300 mg grapiprant unit dosage form of the invention, upon a 14-day oral administration in healthy adult subjects as described herein, provides a urine A_{E}/Dose of about 27.5±about 9.05% on post-dosing day 14. In some embodiments, a 300 mg grapiprant unit dosage form of the invention, upon a 14-day oral administration in healthy adult subjects as described herein, provides a urine A_{E}/Dose of about 27.5±about 4.53% on post-dosing day 14. In some embodiments, a 300 mg grapiprant unit dosage form of the invention, upon a 14-day oral administration in healthy adult subjects as described herein, provides a urine A_{E}/Dose of about 27.5±about 2.26% on post-dosing day 14.

In some embodiments, a 300 mg grapiprant unit dosage form of the invention, upon a 14-day oral administration in healthy adult subjects as described herein, provides a urine CL_{R} of about 44.5±about 10.0 mL/min on post-dosing day 14. In some embodiments, a 300 mg grapiprant unit dosage form of the invention, upon a 14-day oral administration in healthy adult subjects as described herein, provides a urine CL_{R} of about 44.5±about 5.0 mL/min on post-dosing day 14. In some embodiments, a 300 mg grapiprant unit dosage form of the invention, upon a 14-day oral administration in healthy adult subjects as described herein, provides a urine CL_{R} of about 44.5±about 2.5 mL/min on post-dosing day 14.

In some embodiments, a 300 mg grapiprant unit dosage form of the invention, upon a 14-day oral administration in healthy adult subjects as described herein, provides a urine CL_{R}/Fu of about 4838±about 1085 mL/min on post-dosing day 14. In some embodiments, a 300 mg grapiprant unit dosage form of the invention, upon a 14-day oral administration in healthy adult subjects as described herein, provides a urine CL_{R}/Fu of about 4838±about 543 mL/min on post-dosing day 14. In some embodiments, a 300 mg grapiprant unit dosage form of the invention, upon a 14-day oral administration in healthy adult subjects as described herein, provides a urine CL_{R}/Fu of about 4838±about 271 mL/min on post-dosing day 14.

In some embodiments, a 300 mg grapiprant unit dosage form of the invention, upon a 14-day oral administration in healthy adult subjects as described herein, provides one or more pharmacokinetics result on post-dosing days 7 and 14 selected from:
a LS Mean Difference (Least Squares Mean difference of percent change from baseline) of about 16.10% in bone-specific alkaline phosphatase on post-dosing day 7;
a LS Mean Difference of about 19.72% in bone-specific alkaline phosphatase on post-dosing day 14;
a LS Mean change of about −10.30% in blood osteocalcin on post-dosing day 7;
a LS Mean change of about −33.47% in blood osteocalcin on post-dosing day 14;
a LS Mean change of about 12.69% in N-telopeptide of type I collagen on post-dosing day 7; and
a LS Mean change of about −21.33% in N-telopeptide of type I collagen on post-dosing day 14.

In some embodiments, a 300 mg grapiprant unit dosage form of the invention, upon a 14-day oral administration in healthy adult subjects as described herein, provides a LS Mean Difference (Least Squares Mean difference of percent change from baseline) of about 16.10% in bone-specific alkaline phosphatase on post-dosing day 7. In some embodiments, a 300 mg grapiprant unit dosage form of the invention, upon a 14-day oral administration in healthy adult subjects as described herein, provides a LS Mean Difference of about 19.72% in bone-specific alkaline phosphatase on post-dosing day 14. In some embodiments, a 300 mg grapiprant unit dosage form of the invention, upon a 14-day oral administration in healthy adult subjects as described herein, provides a LS Mean change of about −10.30% in blood osteocalcin on post-dosing day 7. In some embodiments, a 300 mg grapiprant unit dosage form of the invention, upon a 14-day oral administration in healthy adult subjects as described herein, provides a LS Mean change of about −33.47% in blood osteocalcin on post-dosing day 14. In some embodiments, a 300 mg grapiprant unit dosage form of the invention, upon a 14-day oral administration in healthy adult subjects as described herein, provides a LS Mean change of about 12.69% in N-telopeptide of type I collagen on post-dosing day 7. In some embodiments, a 300 mg grapiprant unit dosage form of the invention, upon a 14-day oral administration in healthy adult subjects as described herein, provides a LS Mean change of about −21.33% in N-telopeptide of type I collagen on post-dosing day 14.

In some embodiments, a 300 mg grapiprant unit dosage form of the invention, upon a 14-day oral administration in healthy adult subjects as described herein, provides one or more pharmacokinetics result on post-dosing days 7 and 14 selected from:
a LS Mean Difference of about 2.67% to about 29.53% in bone-specific alkaline phosphatase on post-dosing day 7;
a LS Mean Difference of about 6.17% to about 33.28% in bone-specific alkaline phosphatase on post-dosing day 14;
a LS Mean change of about −42.27% to about 21.67% in blood osteocalcin on post-dosing day 7;
a LS Mean change of about −66.18% to about −0.76% in blood osteocalcin on post-dosing day 14;
a LS Mean change of about −25.58% to about 50.96% in N-telopeptide of type I collagen on post-dosing day 7; and
a LS Mean change of about −60.53% to about 17.88% in N-telopeptide of type I collagen on post-dosing day 14.

In some embodiments, a 300 mg grapiprant unit dosage form of the invention, upon a 14-day oral administration in healthy adult subjects as described herein, provides a LS Mean Difference of about 2.67% to about 29.53% in bone-specific alkaline phosphatase on post-dosing day 7. In some embodiments, a 300 mg grapiprant unit dosage form of the invention, upon a 14-day oral administration in healthy adult subjects as described herein, provides a LS Mean Difference of about 6.17% to about 33.28% in bone-specific alkaline phosphatase on post-dosing day 14. In some embodiments, a 300 mg grapiprant unit dosage form of the invention, upon a 14-day oral administration in healthy adult subjects as described herein, provides a LS Mean change of about −42.27% to about 21.67% in blood osteocalcin on post-dosing day 7. In some embodiments, a 300 mg grapiprant unit dosage form of the invention, upon a 14-day oral administration in healthy adult subjects as described herein, provides a LS Mean change of about −66.18% to about −0.76% in blood osteocalcin on post-dosing day 14. In some embodiments, a 300 mg grapiprant unit dosage form of the invention, upon a 14-day oral administration in healthy adult subjects as described herein, provides a LS Mean change of about −25.58% to about 50.96% in N-telopeptide of type I collagen on post-dosing day 7. In some embodiments, a 300 mg grapiprant unit dosage form of the invention, upon a 14-day oral administration in healthy adult subjects as described herein, provides a LS Mean change of about −60.53% to about 17.88% in N-telopeptide of type I collagen on post-dosing day 14.

In some embodiments, a unit dosage form of a pharmaceutical composition comprises about 375 mg of grapiprant, or a pharmaceutically acceptable salt thereof, and one or more pharmaceutically acceptable excipient or carrier (referred to herein as “a 375 mg grapiprant unit dosage form”).

In some embodiments, a 375 mg grapiprant unit dosage form of the invention, upon a single oral administration in a fasted state in healthy adult subjects, provides one or more pharmacokinetics result selected from:

a mean serum AUC (0-tlast) of about 28900 ng*h/mL;

a mean serum AUC (0-inf) of about 29100 ng*h/mL;

a mean serum Cₘₐₓ of about 8990 ng/mL;

a mean serum Tₘₐₓ of about 1 h; and

a mean serum T_{1/2} of about 9.48 h.

In some embodiments, a 375 mg grapiprant unit dosage form of the invention, upon a single oral administration in a fasted state in healthy adult subjects, provides a mean serum AUC (0-tlast) of about 28900 ng*h/mL. In some embodiments, a 375 mg grapiprant unit dosage form of the invention, upon a single oral administration in a fasted state in healthy adult subjects, provides a mean serum AUC (0-inf) of about 29100 ng*h/mL. In some embodiments, a 375 mg grapiprant unit dosage form of the invention, upon a single oral administration in a fasted state in healthy adult subjects, provides a mean serum Cₘₐₓ of about 8990 ng/mL. In some embodiments, a 375 mg grapiprant unit dosage form of the invention, upon a single oral administration in a fasted state in healthy adult subjects, provides a mean serum Tₘₐₓ of about 1 h. In some embodiments, a 375 mg grapiprant unit dosage form of the invention, upon a single oral administration in a fasted state in healthy adult subjects, provides a mean serum T1/2 of about 9.48 h.

In some embodiments, a 375 mg grapiprant unit dosage form of the invention, upon a single oral administration in a fasted state in healthy adult subjects, provides one or more pharmacokinetics result selected from:

a serum AUC (0-tlast) of about 28900±about 13000 ng*h/mL;

a serum AUC (0-inf) of about 29100±about 13000 ng*h/mL;

a serum Cₘₐₓ of about 8990±about 4270 ng/mL;

a serum Tₘₐₓ of about 0.5 to about 3 h; and

a serum T_{1/2} of about 9.48±about 1.87 h.

In some embodiments, a 375 mg grapiprant unit dosage form of the invention, upon a single oral administration in a fasted state in healthy adult subjects, provides a serum AUC (0-tlast) of about 28900±about 13000 ng*h/mL. In some embodiments, a 375 mg grapiprant unit dosage form of the invention, upon a single oral administration in a fasted state in healthy adult subjects, provides a serum AUC (0-tlast) of about 28900±about 6500 ng*h/mL. In some embodiments, a 375 mg grapiprant unit dosage form of the invention, upon a single oral administration in a fasted state in healthy adult subjects, provides a serum AUC (0-tlast) of about 3250±about 13000 ng*h/mL.

In some embodiments, a 375 mg grapiprant unit dosage form of the invention, upon a single oral administration in a fasted state in healthy adult subjects, provides a serum AUC (0-inf) of about 29100±about 13000 ng*h/mL. In some embodiments, a 375 mg grapiprant unit dosage form of the invention, upon a single oral administration in a fasted state in healthy adult subjects, provides a serum AUC (0-inf) of about 29100±about 6500 ng*h/mL. In some embodiments, a 375 mg grapiprant unit dosage form of the invention, upon a single oral administration in a fasted state in healthy adult subjects, provides a serum AUC (0-inf) of about 29100±about 3250 ng*h/mL.

In some embodiments, a 375 mg grapiprant unit dosage form of the invention, upon a single oral administration in a fasted state in healthy adult subjects, provides a serum Cₘₐₓ of about 8990±about 4270 ng/mL. In some embodiments, a 375 mg grapiprant unit dosage form of the invention, upon a single oral administration in a fasted state in healthy adult subjects, provides a serum Cₘₐₓ of about 8990±about 2135 ng/mL. In some embodiments, a 375 mg grapiprant unit dosage form of the invention, upon a single oral administration in a fasted state in healthy adult subjects, provides a serum Cₘₐₓ of about 8990±about 1068 ng/mL.

In some embodiments, a 375 mg grapiprant unit dosage form of the invention, upon a single oral administration in a fasted state in healthy adult subjects, provides a serum Tₘₐₓ of about 0.5 to about 3 h. In some embodiments, a 375 mg grapiprant unit dosage form of the invention, upon a single oral administration in a fasted state in healthy adult subjects, provides a serum Tₘₐₓ of about 0.75 to about 2 h. In some embodiments, a 375 mg grapiprant unit dosage form of the invention, upon a single oral administration in a fasted state in healthy adult subjects, provides a serum Tₘₐₓ of about 0.875 to about 1.5 h.

In some embodiments, a 375 mg grapiprant unit dosage form of the invention, upon a single oral administration in a fasted state in healthy adult subjects, provides a serum T1/2 of about 9.48±about 1.87 h. In some embodiments, a 375 mg grapiprant unit dosage form of the invention, upon a single oral administration in a fasted state in healthy adult subjects, provides a serum T1/2 of about 9.48±about 0.935 h. In some embodiments, a 375 mg grapiprant unit dosage form of the invention, upon a single oral administration in a fasted state in healthy adult subjects, provides a serum T1/2 of about 9.48±about 0.47 h.

In some embodiments, a 375 mg grapiprant unit dosage form of the invention, upon a single oral administration in a fed state in healthy adult subjects, provides one or more pharmacokinetics result selected from:

a mean serum AUC (0-tlast) of about 24600 ng*h/mL;

a mean serum AUC (0-inf) of about 24800 ng*h/mL;

a mean serum Cₘₐₓ of about 5840 ng/mL;

a mean serum Tₘₐₓ of about 4 h; and

a mean serum T_{1/2} of about 8.52 h.

In some embodiments, a 375 mg grapiprant unit dosage form of the invention, upon a single oral administration in a fed state in healthy adult subjects, provides a mean serum AUC (0-tlast) of about 24600 ng*h/mL. In some embodiments, a 375 mg grapiprant unit dosage form of the invention, upon a single oral administration in a fed state in healthy adult subjects, provides a mean serum AUC (0-inf) of about 24800 ng*h/mL. In some embodiments, a 375 mg grapiprant unit dosage form of the invention, upon a single oral administration in a fed state in healthy adult subjects, provides a mean serum Cₘₐₓ of about 5840 ng/mL. In some embodiments, a 375 mg grapiprant unit dosage form of the invention, upon a single oral administration in a fed state in healthy adult subjects, provides a mean serum Tₘₐₓ of about 4 h. In some embodiments, a 375 mg grapiprant unit dosage form of the invention, upon a single oral administration in a fed state in healthy adult subjects, provides a mean serum T_{1/2} of about 8.52 h.

In some embodiments, a 375 mg grapiprant unit dosage form of the invention, upon a single oral administration in a fed state in healthy adult subjects, provides one or more pharmacokinetics result selected from:

a serum AUC (0-tlast) of about 24600±about 8660 ng*h/mL;

a serum AUC (0-inf) of about 24800±about 8720 ng*h/mL;

a serum Cₘₐₓ of about 5840±about 2890 ng/mL;

a serum Tₘₐₓ of about 1.5 to about 4 h; and

a serum T_{1/2} of about 8.52±about 1.29 h.

In some embodiments, a 375 mg grapiprant unit dosage form of the invention, upon a single oral administration in a fed state in healthy adult subjects, provides a serum AUC (0-tlast) of about 24600±about 8660 ng*h/mL. In some embodiments, a 375 mg grapiprant unit dosage form of the invention, upon a single oral administration in a fed state in healthy adult subjects, provides a serum AUC (0-tlast) of about 24600±about 4330 ng*h/mL. In some embodiments, a 375 mg grapiprant unit dosage form of the invention, upon a single oral administration in a fed state in healthy adult subjects, provides a serum AUC (0-tlast) of about 24600±about 2165 ng*h/mL.

In some embodiments, a 375 mg grapiprant unit dosage form of the invention, upon a single oral administration in a fed state in healthy adult subjects, provides a serum AUC (0-inf) of about 24800±about 8720 ng*h/mL. In some embodiments, a 375 mg grapiprant unit dosage form of the invention, upon a single oral administration in a fed state in healthy adult subjects, provides a serum AUC (0-inf) of about 24800±about 4360 ng*h/mL. In some embodiments, a 375 mg grapiprant unit dosage form of the invention, upon a single oral administration in a fed state in healthy adult subjects, provides a serum AUC (0-inf) of about 24800±about 2180 ng*h/mL.

In some embodiments, a 375 mg grapiprant unit dosage form of the invention, upon a single oral administration in a fed state in healthy adult subjects, provides a serum Cₘₐₓ of about 5840±about 2890 ng/mL. In some embodiments, a 375 mg grapiprant unit dosage form of the invention, upon a single oral administration in a fed state in healthy adult subjects, provides a serum Cₘₐₓ of about 5840±about 1445 ng/mL. In some embodiments, a 375 mg grapiprant unit dosage form of the invention, upon a single oral administration in a fed state in healthy adult subjects, provides a serum Cₘₐₓ of about 5840±about 723 ng/mL.

In some embodiments, a 375 mg grapiprant unit dosage form of the invention, upon a single oral administration in a fed state in healthy adult subjects, provides a serum Tₘₐₓ of about 1.5 to about 4 h. In some embodiments, a 375 mg grapiprant unit dosage form of the invention, upon a single oral administration in a fed state in healthy adult subjects, provides a serum Tₘₐₓ of about 2.75 to about 4 h. In some embodiments, a 375 mg grapiprant unit dosage form of the invention, upon a single oral administration in a fed state in healthy adult subjects, provides a serum Tₘₐₓ of about 3.375 to about 4 h.

In some embodiments, a 375 mg grapiprant unit dosage form of the invention, upon a single oral administration in a fed state in healthy adult subjects, provides a serum T1/2 of about 8.52±about 1.29 h. In some embodiments, a 375 mg grapiprant unit dosage form of the invention, upon a single oral administration in a fed state in healthy adult subjects, provides a serum T1/2 of about 8.52±about 0.645 h. In some embodiments, a 375 mg grapiprant unit dosage form of the invention, upon a single oral administration in a fed state in healthy adult subjects, provides a serum T1/2 of about 8.52±about 0.32 h.

### 4. Description of Exemplary Dosage Forms

A unit dosage form of the invention can be formulated for oral administration. Pharmaceutical compositions/formulations that are suitable for oral administration can be provided as discrete dosage forms, such as, but not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In some embodiments, such dosage forms contain predetermined amounts of active ingredients, and may be prepared by methods of pharmacy known to those skilled in the art. See generally, Remington's Pharmaceutical Sciences, 18th ed., Mack Publishing, Easton Pa. (1990). As used herein, oral administration also includes buccal, lingual, and sublingual administration.

In some embodiments, a unit dosage form of the invention is a tablet. In some embodiments, a unit dosage form of the invention is a capsule. In some embodiments, a unit dosage form of the invention is a caplet.

In some embodiments, a unit dosage form provided herein are prepared by combining the active ingredients in an intimate admixture with one or more pharmaceutically acceptable excipient or carrier, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide, according to conventional pharmaceutical compounding techniques. Excipients or carriers can take a wide variety of forms depending on the form of preparation desired for administration. For example, excipients or carriers suitable for use in oral liquid or aerosol dosage forms include, but are not limited to, water, glycols, oils, alcohols, flavoring agents, preservatives, and coloring agents. Examples of excipients or carriers suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

In certain embodiments, the dosage form is a tablet, wherein the tablet is manufactured using standard, art-recognized tablet processing procedures and equipment. In certain embodiments, the method for forming the tablets is direct compression of a powdered, crystalline and/or granular composition comprising a solid form provided herein, alone or in combination with one or more excipients or carriers, such as, for example, carriers, additives, polymers, or the like. In certain embodiments, as an alternative to direct compression, the tablets may be prepared using wet granulation or dry granulation processes. In certain embodiments, the tablets are molded rather than compressed, starting with a moist or otherwise tractable material. In certain embodiments, compression and granulation techniques are used.

In certain embodiments, the dosage form is a capsule, wherein the capsules may be manufactured using standard, art-recognized capsule processing procedures and equipments. In certain embodiments, soft gelatin capsules may be prepared in which the capsules contain a mixture comprising a solid form provided herein and vegetable oil or non-aqueous, water miscible materials, such as, for example, polyethylene glycol and the like. In certain embodiments, hard gelatin capsules may be prepared containing granules of solid forms provided herein in combination with a solid pulverulent carrier, such as, for example, lactose, saccharose, sorbitol, mannitol, potato starch, corn starch, amylopectin, cellulose derivatives, or gelatin. In certain embodiments, a hard gelatin capsule shell may be prepared from a capsule composition comprising gelatin and a small amount of plasticizer such as glycerol. In certain embodiments, as an alternative to gelatin, the capsule shell may be made of a carbohydrate material. In certain embodiments, the capsule composition may additionally include polymers, colorings, flavorings and opacifiers as required. In certain embodiments, the capsule comprises HPMC.

Examples of excipients or carriers that can be used in oral dosage forms provided herein include, but are not limited to, diluents (bulking agents), lubricants, disintegrants, fillers, stabilizers, surfactants, preservatives, coloring agents, flavoring agents, binding agents (binders), excipient supports, glidants, permeation enhancement excipients, plasticizers and the like, e.g., as known in the art. It will be understood by those in the art that some substances serve more than one purpose in a pharmaceutical composition. For instance, some substances are binders that help hold a tablet together after compression, yet are also disintegrants that help break the tablet apart once it reaches the target delivery site. Selection of excipients and amounts to use may be readily determined by the formulation scientist based upon experience and consideration of standard procedures and reference works available in the art.

In certain embodiments, dosage forms provided herein comprise one or more binders. Binders may be used, e.g., to impart cohesive qualities to a tablet or a capsule, and thus ensure that the formulation remains intact after compression. Suitable binders include, but are not limited to, starch (including potato starch, corn starch, and pregelatinized starch), gelatin, sugars (including sucrose, glucose, dextrose and lactose), polyethylene glycol, propylene glycol, waxes, and natural and synthetic gums, e.g., acacia sodium alginate, polyvinylpyrrolidone (PVP), cellulosic polymers (including hydroxypropyl cellulose (HPC), hydroxypropylmethylcellulose (HPMC), methyl cellulose, ethyl cellulose, hydroxyethyl cellulose (HEC), carboxymethyl cellulose and the like), veegum, carbomer (e.g., carbopol), sodium, dextrin, guar gum, hydrogenated vegetable oil, magnesium aluminum silicate, maltodextrin, polymethacrylates, povidone (e.g., KOLLIDON, PLASDONE), microcrystalline cellulose, among others. Binding agents also include, e.g., acacia, agar, alginic acid, cabomers, carrageenan, cellulose acetate phthalate, ceratonia, chitosan, confectioner's sugar, copovidone, dextrates, dextrin, dextrose, ethylcellulose, gelatin, glyceryl behenate, guar gum, hydroxyethyl cellulose, hydroxyethylmethyl cellulose, hydroxypropyl cellulose, hydroxypropyl starch, hypromellose, inulin, lactose, magnesium aluminum silicate, maltodextrin, maltose, methylcellulose, poloxamer, polycarbophil, polydextrose, polyethylene oxide, polymethylacrylates, povidone, sodium alginate, sodium carboxymethylcellulose, starch, pregelatinized starch, stearic acid, sucrose, and zein.

Suitable forms of microcrystalline cellulose include, but are not limited to, the materials sold as AVICEL-PH-101, AVICEL-PH-103 AVICEL RC-581, AVICEL-PH-105 (FMC Corporation, Marcus Hook, Pa.), and mixtures thereof. In some embodiment, a specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

In certain embodiments, dosage forms provided herein comprise one or more diluents. Diluents may be used, e.g., to increase bulk so that a practical size tablet or capsule is ultimately provided. Suitable diluents include dicalcium phosphate, calcium sulfate, lactose, cellulose, kaolin, mannitol, sodium chloride, dry starch, microcrystalline cellulose (e.g., AVICEL), microfine cellulose, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT), potassium chloride, sodium chloride, sorbitol and talc, among others. Diluents also include, e.g., ammonium alginate, calcium carbonate, calcium phosphate, calcium sulfate, cellulose acetate, compressible sugar, confectioner's sugar, dextrates, dextrin, dextrose, erythritol, ethylcellulose, fructose, fumaric acid, glyceryl palmitostearate, isomalt, kaolin, lacitol, lactose, mannitol, magnesium carbonate, magnesium oxide, maltodextrin, maltose, medium-chain triglycerides, microcrystalline cellulose, microcrystalline silicified cellulose, powered cellulose, polydextrose, polymethylacrylates, simethicone, sodium alginate, sodium chloride, sorbitol, starch, pregelatinized starch, sucrose, sulfobutylether-.beta.-cyclodextrin, talc, tragacanth, trehalose, and xylitol.

Disintegrants may be used in the compositions to provide tablets or capsules that disintegrate when exposed to an aqueous environment. Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof.

Lubricants that can be used in pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof.

Suitable glidants include, but are not limited to, colloidal silicon dioxide, CAB-O-SIL® (Cabot Co. of Boston, Mass.), and asbestos-free talc.

Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye.

Suitable flavoring agents include, but are not limited to, natural flavors extracted from plants, such as fruits, and synthetic blends of compounds which produce a pleasant taste sensation, such as peppermint and methyl salicylate.

Suitable sweetening agents include, but are not limited to, sucrose, lactose, mannitol, syrups, glycerin, and artificial sweeteners, such as saccharin and aspartame.

Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate.

Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone.

Suitable preservatives include, but are not limited to, glycerin, methyl and propylparaben, benzoic add, sodium benzoate and alcohol.

Suitable wetting agents include, but are not limited to, propylene glycol monostearate, sorbitan monooleate, diethylene glycol monolaurate, and polyoxyethylene lauryl ether.

Suitable solvents include, but are not limited to, glycerin, sorbitol, ethyl alcohol, and syrup.

Suitable non-aqueous liquids utilized in emulsions include, but are not limited to, mineral oil and cottonseed oil.

Suitable organic acids include, but are not limited to, citric and tartaric acid.

Suitable sources of carbon dioxide include, but are not limited to, sodium bicarbonate and sodium carbonate.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

A tablet dosage form can be prepared from the active ingredient in powdered, crystalline, or granular forms, alone or in combination with one or more carriers or excipients described herein, including binders, disintegrants, controlled-release polymers, lubricants, diluents, and/or colorants.

In some embodiments, a unit dosage form of the invention comprises one or more pharmaceutically acceptable excipient selected from microcrystalline cellulose, lactose monohydrate (modified), croscarmellose sodium, hydroxypropyl cellulose, and magnesium stearate.

In some embodiments, a unit dosage form of the invention comprises microcrystalline cellulose at the amount of about 1.82 mg per mg of grapiprant.

In some embodiments, a unit dosage form of the invention comprises lactose monohydrate (modified) at the amount of about 0.88 mg per mg of grapiprant.

In some embodiments, a unit dosage form of the invention comprises croscarmellose sodium at the amount of about 0.2 mg per mg of grapiprant.

In some embodiments, a unit dosage form of the invention comprises hydroxypropyl cellulose at the amount of about 0.08 mg per mg of grapiprant.

In some embodiments, a unit dosage form of the invention comprises magnesium stearate at the amount of about 0.02 mg per mg of grapiprant.

In some embodiments, a unit dosage form of the invention comprises about 50 mg of grapiprant, about 91 mg of microcrystalline cellulose, about 44.0 mg of lactose monohydrate (modified), about 10.0 mg of croscarmellose sodium, about 4.0 mg of hydroxypropyl cellulose, and about 1 mg of magnesium stearate.

In some embodiments, a unit dosage form of the invention comprises about 150 mg of grapiprant, about 273 mg of microcrystalline cellulose, about 132.0 mg of lactose monohydrate (modified), about 30.0 mg of croscarmellose sodium, about 12.0 mg of hydroxypropyl cellulose, and about 3 mg of magnesium stearate.

In some embodiments, a unit dosage form of the invention comprises about 250 mg of grapiprant, about 455 mg of microcrystalline cellulose, about 220.0 mg of lactose monohydrate (modified), about 50.0 mg of croscarmellose sodium, 20.0 mg of hydroxypropyl cellulose, and about 5 mg of magnesium stearate.

In some embodiments, a unit dosage form of the invention comprises about 300 mg of grapiprant, about 546 mg of microcrystalline cellulose, about 264.0 mg of lactose monohydrate (modified), about 60.0 mg of croscarmellose sodium, about 24.0 mg of hydroxypropyl cellulose, and about 6 mg of magnesium stearate.

In some embodiments, a unit dosage form of the invention comprises about 375 mg of grapiprant, about 682.5 mg of microcrystalline cellulose, about 330.0 mg of lactose monohydrate (modified), about 75.0 mg of croscarmellose sodium, about 30.0 mg of hydroxypropyl cellulose, and about 7.5 mg of magnesium stearate.

In some embodiments, a unit dosage form of the invention comprises one or more tablets. In some embodiments, one or more tablet comprises about 125 mg of grapiprant, about 227.5 mg of microcrystalline cellulose, about 110.0 mg of lactose monohydrate (modified), about 25.0 mg of croscarmellose sodium, 10.0 mg of hydroxypropyl cellulose, and about 2.5 mg of magnesium stearate. In some embodiments, one or more tablet comprises about 25 mg of grapiprant, about 45.5 mg of microcrystalline cellulose, about 22.0 mg of lactose monohydrate (modified), about 5.0 mg of croscarmellose sodium, about 2.0 mg of hydroxypropyl cellulose, and about 0.5 mg of magnesium stearate.

### 5. Description of Exemplary Unit Dosage Form OPC and Liquid Unit Dosage Forms

In some embodiments, the present invention provides a unit dosage form of a pharmaceutical composition, in OPC formulation, comprising about 1 mg to about 2000 mg grapiprant, or a pharmaceutically acceptable salt thereof. In some embodiments, the present invention provides a unit dosage form of a pharmaceutical composition, in OPC formulation, comprising about 10 mg to about 1500 mg grapiprant, or a pharmaceutically acceptable salt thereof. In some embodiments, the present invention provides a unit dosage form of a pharmaceutical composition, in OPC formulation, comprising about 30 mg to about 1000 mg grapiprant, or a pharmaceutically acceptable salt thereof. In some embodiments, the present invention provides a unit dosage form of a pharmaceutical composition, in OPC formulation, comprising about 100 mg to about 600 mg grapiprant, or a pharmaceutically acceptable salt thereof.

In some embodiments, a unit dosage form of the invention in OPC formulation comprises about 1 mg grapiprant, or a pharmaceutically acceptable salt thereof (referred to herein as “a 1 mg grapiprant unit dosage form OPC”). In some embodiments, a unit dosage form of the invention in OPC formulation comprises about 3 mg grapiprant, or a pharmaceutically acceptable salt thereof (referred to herein as “a 3 mg grapiprant unit dosage form OPC”). In some embodiments, a unit dosage form of the invention in OPC formulation comprises about 10 mg grapiprant, or a pharmaceutically acceptable salt thereof (referred to herein as “a 10 mg grapiprant unit dosage form OPC”). In some embodiments, a unit dosage form of the invention in OPC formulation comprises about 30 mg grapiprant, or a pharmaceutically acceptable salt thereof (referred to herein as “a 30 mg grapiprant unit dosage form OPC”). In some embodiments, a unit dosage form of the invention in OPC formulation comprises about 60 mg grapiprant, or a pharmaceutically acceptable salt thereof (referred to herein as “a 60 mg grapiprant unit dosage form OPC”). In some embodiments, a unit dosage form of the invention in OPC formulation comprises about 100 mg grapiprant, or a pharmaceutically acceptable salt thereof (referred to herein as “a 100 mg grapiprant unit dosage form OPC”). In some embodiments, a unit dosage form of the invention in OPC formulation comprises about 200 mg grapiprant, or a pharmaceutically acceptable salt thereof (referred to herein as “a 200 mg grapiprant unit dosage form OPC”). In some embodiments, a unit dosage form of the invention in OPC formulation comprises about 300 mg grapiprant, or a pharmaceutically acceptable salt thereof (referred to herein as “a 300 mg grapiprant unit dosage form OPC”). In some embodiments, a unit dosage form of the invention in OPC formulation comprises about 600 mg grapiprant, or a pharmaceutically acceptable salt thereof (referred to herein as “a 600 mg grapiprant unit dosage form OPC”). In some embodiments, a unit dosage form of the invention in OPC formulation comprises about 1000 mg grapiprant, or a pharmaceutically acceptable salt thereof (referred to herein as “a 1000 mg grapiprant unit dosage form OPC”). In some embodiments, a unit dosage form of the invention in OPC formulation comprises about 1500 mg grapiprant, or a pharmaceutically acceptable salt thereof (referred to herein as “a 1500 mg grapiprant unit dosage form OPC”). In some embodiments, a unit dosage form of the invention in OPC formulation comprises about 2000 mg grapiprant, or a pharmaceutically acceptable salt thereof (referred to herein as “a 2000 mg grapiprant unit dosage form OPC”).

In some embodiments, the present invention provides a unit dosage form of a pharmaceutical composition, in liquid form, comprising about 1 mg to about 2000 mg grapiprant, or a pharmaceutically acceptable salt thereof, and water. In some embodiments, the present invention provides a unit dosage form of a pharmaceutical composition, in liquid form, comprising about 10 mg to about 1500 mg grapiprant, or a pharmaceutically acceptable salt thereof, and water. In some embodiments, the present invention provides a unit dosage form of a pharmaceutical composition, in liquid form, comprising about 30 mg to about 1000 mg grapiprant, or a pharmaceutically acceptable salt thereof, and water. In some embodiments, the present invention provides a unit dosage form of a pharmaceutical composition, in liquid form, comprising about 100 mg to about 600 mg grapiprant, or a pharmaceutically acceptable salt thereof, and water.

In some embodiments, a unit dosage form of the invention in liquid form comprises about 1 mg grapiprant, or a pharmaceutically acceptable salt thereof, and water (referred to herein as “a 1 mg grapiprant liquid unit dosage form”). In some embodiments, a unit dosage form of the invention in liquid form comprises about 3 mg grapiprant, or a pharmaceutically acceptable salt thereof, and water (referred to herein as “a 3 mg grapiprant liquid unit dosage form”). In some embodiments, a unit dosage form of the invention in liquid form comprises about 10 mg grapiprant, or a pharmaceutically acceptable salt thereof, and water (referred to herein as “a 10 mg grapiprant liquid unit dosage form”). In some embodiments, a unit dosage form of the invention in liquid form comprises about 30 mg grapiprant, or a pharmaceutically acceptable salt thereof, and water (referred to herein as “a 30 mg grapiprant liquid unit dosage form”). In some embodiments, a unit dosage form of the invention in liquid form comprises about 60 mg grapiprant, or a pharmaceutically acceptable salt thereof, and water (referred to herein as “a 60 mg grapiprant liquid unit dosage form”). In some embodiments, a unit dosage form of the invention in liquid form comprises about 100 mg grapiprant, or a pharmaceutically acceptable salt thereof, and water (referred to herein as “a 100 mg grapiprant liquid unit dosage form”). In some embodiments, a unit dosage form of the invention in liquid form comprises about 200 mg grapiprant, or a pharmaceutically acceptable salt thereof, and water (referred to herein as “a 200 mg grapiprant liquid unit dosage form”). In some embodiments, a unit dosage form of the invention in liquid form comprises about 300 mg grapiprant, or a pharmaceutically acceptable salt thereof, and water (referred to herein as “a 300 mg grapiprant liquid unit dosage form”). In some embodiments, a unit dosage form of the invention in liquid form comprises about 600 mg grapiprant, or a pharmaceutically acceptable salt thereof, and water (referred to herein as “a 600 mg grapiprant liquid unit dosage form”). In some embodiments, a unit dosage form of the invention in liquid form comprises about 1000 mg grapiprant, or a pharmaceutically acceptable salt thereof, and water (referred to herein as “a 1000 mg grapiprant liquid unit dosage form”). In some embodiments, a unit dosage form of the invention in liquid form comprises about 1500 mg grapiprant, or a pharmaceutically acceptable salt thereof, and water (referred to herein as “a 1500 mg grapiprant liquid unit dosage form”). In some embodiments, a unit dosage form of the invention in liquid form comprises about 2000 mg grapiprant, or a pharmaceutically acceptable salt thereof, and water (referred to herein as “a 2000 mg grapiprant liquid unit dosage form”).

In some embodiments, a grapiprant liquid unit dosage form of the invention comprises about 50-150 mL, 62.5-137.5 mL, 75-125 mL, or 87.5-112.5 mL water. In some embodiments, a grapiprant liquid unit dosage form of the invention comprises about 100 mL water.

In some embodiments, a 1 mg grapiprant liquid unit dosage form of the invention, upon a single oral administration in a fasted state in healthy adult subjects, provides one or more pharmacokinetics result selected from:

a mean serum Cₘₐₓ of about 6.59 ng/mL;

a mean serum Tₘₐₓ of about 2.5 h; and

a mean serum AUC(Tlast) of about 59.1 ng*h/mL.

In some embodiments, a 1 mg grapiprant liquid unit dosage form of the invention, upon a single oral administration in a fasted state in healthy adult subjects, provides a mean serum Cmax of about 6.59 ng/mL. In some embodiments, a 1 mg grapiprant liquid unit dosage form of the invention, upon a single oral administration in a fasted state in healthy adult subjects, provides a mean serum Tmax of about 2.5 h. In some embodiments, a 1 mg grapiprant liquid unit dosage form of the invention, upon a single oral administration in a fasted state in healthy adult subjects, provides a mean serum AUC(Tlast) of about 59.1 ng*h/mL.

In some embodiments, a 1 mg grapiprant liquid unit dosage form of the invention, upon a single oral administration in a fasted state in healthy adult subjects, provides one or more pharmacokinetics result selected from:

a serum Cₘₐₓ of about 6.59±about 1.86 ng/mL;

a serum Tₘₐₓ of about 1.5 h to about 8.00 h; and

a serum AUC(Tlast) of about 59.1±about 18.8 ng*h/mL.

In some embodiments, a 1 mg grapiprant liquid unit dosage form of the invention, upon a single oral administration in a fasted state in healthy adult subjects, provides a serum Cₘₐₓ of about 6.59±about 1.86 ng/mL. In some embodiments, a 1 mg grapiprant liquid unit dosage form of the invention, upon a single oral administration in a fasted state in healthy adult subjects, provides a serum Cₘₐₓ of about 6.59±about 0.93 ng/mL. In some embodiments, a 1 mg grapiprant liquid unit dosage form of the invention, upon a single oral administration in a fasted state in healthy adult subjects, provides a serum Cₘₐₓ of about 6.59±about 0.47 ng/mL.

In some embodiments, a 1 mg grapiprant liquid unit dosage form of the invention, upon a single oral administration in a fasted state in healthy adult subjects, provides a serum Tₘₐₓ of about 1.5 h to about 8.00 h. In some embodiments, a 1 mg grapiprant liquid unit dosage form of the invention, upon a single oral administration in a fasted state in healthy adult subjects, provides a serum Tₘₐₓ of about 2.0 h to about 5.25 h. In some embodiments, a 1 mg grapiprant liquid unit dosage form of the invention, upon a single oral administration in a fasted state in healthy adult subjects, provides a serum Tₘₐₓ of about 1.75 h to about 3.875 h.

In some embodiments, a 1 mg grapiprant liquid unit dosage form of the invention, upon a single oral administration in a fasted state in healthy adult subjects, provides a serum AUC(Tlast) of about 59.1±about 18.8 ng*h/mL. In some embodiments, a 1 mg grapiprant liquid unit dosage form of the invention, upon a single oral administration in a fasted state in healthy adult subjects, provides a serum AUC(Tlast) of about 59.1±about 9.4 ng*h/mL. In some embodiments, a 1 mg grapiprant liquid unit dosage form of the invention, upon a single oral administration in a fasted state in healthy adult subjects, provides a serum AUC(Tlast) of about 59.1±about 4.7 ng*h/mL.

In some embodiments, a 3 mg grapiprant liquid unit dosage form of the invention, upon a single oral administration in a fasted state in healthy adult subjects, provides one or more pharmacokinetics result selected from:

a mean serum Cₘₐₓ of about 23.1 ng/mL;

a mean serum Tₘₐₓ of about 3.00 h;

a mean serum AUC(Tlast) of about 246 ng*h/mL;

a mean serum AUC(inf) of about 260 ng*h/mL; and

a mean serum T_{1/2} of about 5.34 h.

In some embodiments, a 3 mg grapiprant liquid unit dosage form of the invention, upon a single oral administration in a fasted state in healthy adult subjects, provides a mean serum Cmax of about 23.1 ng/mL. In some embodiments, a 3 mg grapiprant liquid unit dosage form of the invention, upon a single oral administration in a fasted state in healthy adult subjects, provides a mean serum Tmax of about 3.00 h. In some embodiments, a 3 mg grapiprant liquid unit dosage form of the invention, upon a single oral administration in a fasted state in healthy adult subjects, provides a mean serum AUC(Tlast) of about 246 ng*h/mL. In some embodiments, a 3 mg grapiprant liquid unit dosage form of the invention, upon a single oral administration in a fasted state in healthy adult subjects, provides a mean serum AUC(inf) of about 260 ng*h/mL. In some embodiments, a 3 mg grapiprant liquid unit dosage form of the invention, upon a single oral administration in a fasted state in healthy adult subjects, provides a mean serum T1/2 of about 5.34 h.

In some embodiments, a 3 mg grapiprant liquid unit dosage form of the invention, upon a single oral administration in a fasted state in healthy adult subjects, provides one or more pharmacokinetics result selected from:

a serum Cₘₐₓ of about 23.1±about 6.16 ng/mL;

a serum Tₘₐₓ of about 2.50 h to about 6.00 h;

a serum AUC(Tlast) of about 246±about 76.7 ng*h/mL;

a serum AUC(inf) of about 260±about 82.7 ng*h/mL; and

a serum T1/2 of about 5.34±about 0.147 h.

In some embodiments, a 3 mg grapiprant liquid unit dosage form of the invention, upon a single oral administration in a fasted state in healthy adult subjects, provides a serum Cₘₐₓ of about 23.1±about 6.16 ng/mL. In some embodiments, a 3 mg grapiprant liquid unit dosage form of the invention, upon a single oral administration in a fasted state in healthy adult subjects, provides a serum Cₘₐₓ of about 23.1±about 3.08 ng/mL. In some embodiments, a 3 mg grapiprant liquid unit dosage form of the invention, upon a single oral administration in a fasted state in healthy adult subjects, provides a serum Cₘₐₓ of about 23.1±about 1.54 ng/mL.

In some embodiments, a 3 mg grapiprant liquid unit dosage form of the invention, upon a single oral administration in a fasted state in healthy adult subjects, provides a serum Tₘₐₓ of about 2.50 h to about 6.00 h. In some embodiments, a 3 mg grapiprant liquid unit dosage form of the invention, upon a single oral administration in a fasted state in healthy adult subjects, provides a serum Tₘₐₓ of about 2.75 h to about 4.50 h. In some embodiments, a 3 mg grapiprant liquid unit dosage form of the invention, upon a single oral administration in a fasted state in healthy adult subjects, provides a serum Tₘₐₓ of about 2.875 h to about 3.25 h.

In some embodiments, a 3 mg grapiprant liquid unit dosage form of the invention, upon a single oral administration in a fasted state in healthy adult subjects, provides a serum AUC(Tlast) of about 246±about 76.7 ng*h/mL. In some embodiments, a 3 mg grapiprant liquid unit dosage form of the invention, upon a single oral administration in a fasted state in healthy adult subjects, provides a serum AUC(Tlast) of about 246±about 38.35 ng*h/mL. In some embodiments, a 3 mg grapiprant liquid unit dosage form of the invention, upon a single oral administration in a fasted state in healthy adult subjects, provides a serum AUC(Tlast) of about 246±about 19.175 ng*h/mL.

In some embodiments, a 3 mg grapiprant liquid unit dosage form of the invention, upon a single oral administration in a fasted state in healthy adult subjects, provides a serum AUC(inf) of about 260±about 82.7 ng*h/mL. In some embodiments, a 3 mg grapiprant liquid unit dosage form of the invention, upon a single oral administration in a fasted state in healthy adult subjects, provides a serum AUC(inf) of about 260±about 41.35 ng*h/mL. In some embodiments, a 3 mg grapiprant liquid unit dosage form of the invention, upon a single oral administration in a fasted state in healthy adult subjects, provides a serum AUC(inf) of about 260±about 20.675 ng*h/mL.

In some embodiments, a 3 mg grapiprant liquid unit dosage form of the invention, upon a single oral administration in a fasted state in healthy adult subjects, provides a serum T1/2 of about 5.34±about 0.147 h. In some embodiments, a 3 mg grapiprant liquid unit dosage form of the invention, upon a single oral administration in a fasted state in healthy adult subjects, provides a serum T1/2 of about 5.34±about 0.074 h. In some embodiments, a 3 mg grapiprant liquid unit dosage form of the invention, upon a single oral administration in a fasted state in healthy adult subjects, provides a serum T1/2 of about 5.34±about 0.037 h.

In some embodiments, a 10 mg grapiprant liquid unit dosage form of the invention, upon a single oral administration in a fasted state in healthy adult subjects, provides one or more pharmacokinetics result selected from:

a mean serum Cₘₐₓ of about 110 ng/mL;

a mean serum Tₘₐₓ of about 2.00 h;

a mean serum AUC(Tlast) of about 901 ng*h/mL;

a mean serum AUC(inf) of about 917 ng*h/mL; and

a mean serum T_{1/2} of about 5.10 h.

In some embodiments, a 10 mg grapiprant liquid unit dosage form of the invention, upon a single oral administration in a fasted state in healthy adult subjects, provides a mean serum Cmax of about 110 ng/mL. In some embodiments, a 10 mg grapiprant liquid unit dosage form of the invention, upon a single oral administration in a fasted state in healthy adult subjects, provides a mean serum Tₘₐₓ of about 2.00 h. In some embodiments, a 10 mg grapiprant liquid unit dosage form of the invention, upon a single oral administration in a fasted state in healthy adult subjects, provides a mean serum AUC(Tlast) of about 901 ng*h/mL. In some embodiments, a 10 mg grapiprant liquid unit dosage form of the invention, upon a single oral administration in a fasted state in healthy adult subjects, provides a mean serum AUC(inf) of about 917 ng*h/mL. In some embodiments, a 10 mg grapiprant liquid unit dosage form of the invention, upon a single oral administration in a fasted state in healthy adult subjects, provides a mean serum T1/2 of about 5.10 h.

In some embodiments, a 10 mg grapiprant liquid unit dosage form of the invention, upon a single oral administration in a fasted state in healthy adult subjects, provides one or more pharmacokinetics result selected from:

a serum Cₘₐₓ of about 110±about 21.0 ng/mL;

a serum Tₘₐₓ of about 1.00 h to about 2.50 h;

a serum AUC(Tlast) of about 901±about 179 ng*h/mL;

a serum AUC(inf) of about 917±about 178 ng*h/mL; and

a serum T_{1/2} of about 5.10±about 1.90 h.

In some embodiments, a 10 mg grapiprant liquid unit dosage form of the invention, upon a single oral administration in a fasted state in healthy adult subjects, provides a serum Cₘₐₓ of about 110±about 21.0 ng/mL. In some embodiments, a 10 mg grapiprant liquid unit dosage form of the invention, upon a single oral administration in a fasted state in healthy adult subjects, provides a serum Cₘₐₓ of about 110±about 10.50 ng/mL. In some embodiments, a 10 mg grapiprant liquid unit dosage form of the invention, upon a single oral administration in a fasted state in healthy adult subjects, provides a serum Cₘₐₓ of about 110±about 5.25 ng/mL.

In some embodiments, a 10 mg grapiprant liquid unit dosage form of the invention, upon a single oral administration in a fasted state in healthy adult subjects, provides a serum Tₘₐₓ of about 1.00 h to about 2.50 h. In some embodiments, a 10 mg grapiprant liquid unit dosage form of the invention, upon a single oral administration in a fasted state in healthy adult subjects, provides a serum Tₘₐₓ of about 1.50 h to about 2.25 h. In some embodiments, a 10 mg grapiprant liquid unit dosage form of the invention, upon a single oral administration in a fasted state in healthy adult subjects, provides a serum Tₘₐₓ of about 1.75 h to about 2.125 h.

In some embodiments, a 10 mg grapiprant liquid unit dosage form of the invention, upon a single oral administration in a fasted state in healthy adult subjects, provides a serum AUC(Tlast) of about 901±about 179 ng*h/mL. In some embodiments, a 10 mg grapiprant liquid unit dosage form of the invention, upon a single oral administration in a fasted state in healthy adult subjects, provides a serum AUC(Tlast) of about 901±about 89.5 ng*h/mL. In some embodiments, a 10 mg grapiprant liquid unit dosage form of the invention, upon a single oral administration in a fasted state in healthy adult subjects, provides a serum AUC(Tlast) of about 901±about 44.75 ng*h/mL.

In some embodiments, a 10 mg grapiprant liquid unit dosage form of the invention, upon a single oral administration in a fasted state in healthy adult subjects, provides a serum AUC(inf) of about 917±about 178 ng*h/mL. In some embodiments, a 10 mg grapiprant liquid unit dosage form of the invention, upon a single oral administration in a fasted state in healthy adult subjects, provides a serum AUC(inf) of about 917±about 89 ng*h/mL. In some embodiments, a 10 mg grapiprant liquid unit dosage form of the invention, upon a single oral administration in a fasted state in healthy adult subjects, provides a serum AUC(inf) of about 917±about 44.5 ng*h/mL.

In some embodiments, a 10 mg grapiprant liquid unit dosage form of the invention, upon a single oral administration in a fasted state in healthy adult subjects, provides a serum T1/2 of about 5.10±about 1.90 h. In some embodiments, a 10 mg grapiprant liquid unit dosage form of the invention, upon a single oral administration in a fasted state in healthy adult subjects, provides a serum T1/2 of about 5.10±about 0.95 h. In some embodiments, a 10 mg grapiprant liquid unit dosage form of the invention, upon a single oral administration in a fasted state in healthy adult subjects, provides a serum T1/2 of about 5.10±about 0.475 h.

In some embodiments, a 30 mg grapiprant liquid unit dosage form of the invention, upon a single oral administration in a fasted state in healthy adult subjects, provides one or more pharmacokinetics result selected from:

a mean serum Cₘₐₓ of about 262 ng/mL;

a mean serum Tₘₐₓ of about 1.00 h;

a mean serum AUC(Tlast) of about 2120 ng*h/mL;

a mean serum AUC(inf) of about 2150 ng*h/mL; and

a mean serum T1/2 of about 6.14 h.

In some embodiments, a 30 mg grapiprant liquid unit dosage form of the invention, upon a single oral administration in a fasted state in healthy adult subjects, provides a mean serum Cₘₐₓ of about 262 ng/mL. In some embodiments, a 30 mg grapiprant liquid unit dosage form of the invention, upon a single oral administration in a fasted state in healthy adult subjects, provides a mean serum Tₘₐₓ of about 1.00 h. In some embodiments, a 30 mg grapiprant liquid unit dosage form of the invention, upon a single oral administration in a fasted state in healthy adult subjects, provides a mean serum AUC(Tlast) of about 2120 ng*h/mL. In some embodiments, a 30 mg grapiprant liquid unit dosage form of the invention, upon a single oral administration in a fasted state in healthy adult subjects, provides a mean serum AUC(inf) of about 2150 ng*h/mL. In some embodiments, a 30 mg grapiprant liquid unit dosage form of the invention, upon a single oral administration in a fasted state in healthy adult subjects, provides a mean serum T1/2 of about 6.14 h.

In some embodiments, a 30 mg grapiprant liquid unit dosage form of the invention, upon a single oral administration in a fasted state in healthy adult subjects, provides one or more pharmacokinetics result selected from:

a serum Cₘₐₓ of about 262±about 83.1 ng/mL;

a serum Tₘₐₓ of about 1.00 to about 2.00 h;

a serum AUC(Tlast) of about 2120±about 750 ng*h/mL;

a serum AUC(inf) of about 2150±about 740 ng*h/mL; and

a serum T1/2 of about 6.14±about 1.36 h.

In some embodiments, a 30 mg grapiprant liquid unit dosage form of the invention, upon a single oral administration in a fasted state in healthy adult subjects, provides a serum Cₘₐₓ of about 262±about 83.1 ng/mL. In some embodiments, a 30 mg grapiprant liquid unit dosage form of the invention, upon a single oral administration in a fasted state in healthy adult subjects, provides a serum Cₘₐₓ of about 262±about 41.55 ng/mL. In some embodiments, a 30 mg grapiprant liquid unit dosage form of the invention, upon a single oral administration in a fasted state in healthy adult subjects, provides a serum Cₘₐₓ of about 262±about 20.775 ng/mL.

In some embodiments, a 30 mg grapiprant liquid unit dosage form of the invention, upon a single oral administration in a fasted state in healthy adult subjects, provides a serum Tₘₐₓ of about 1.00 to about 2.00 h. In some embodiments, a 30 mg grapiprant liquid unit dosage form of the invention, upon a single oral administration in a fasted state in healthy adult subjects, provides a serum Tₘₐₓ of about 1.00 to about 1.50 h. In some embodiments, a 30 mg grapiprant liquid unit dosage form of the invention, upon a single oral administration in a fasted state in healthy adult subjects, provides a serum Tₘₐₓ of about 1.00 to about 1.25 h.

In some embodiments, a 30 mg grapiprant liquid unit dosage form of the invention, upon a single oral administration in a fasted state in healthy adult subjects, provides a serum AUC(Tlast) of about 2120±about 750 ng*h/mL. In some embodiments, a 30 mg grapiprant liquid unit dosage form of the invention, upon a single oral administration in a fasted state in healthy adult subjects, provides a serum AUC(Tlast) of about 2120±about 375 ng*h/mL. In some embodiments, a 30 mg grapiprant liquid unit dosage form of the invention, upon a single oral administration in a fasted state in healthy adult subjects, provides a serum AUC(Tlast) of about 2120±about 187.5 ng*h/mL.

In some embodiments, a 30 mg grapiprant liquid unit dosage form of the invention, upon a single oral administration in a fasted state in healthy adult subjects, provides a serum AUC(inf) of about 2150±about 740 ng*h/mL. In some embodiments, a 30 mg grapiprant liquid unit dosage form of the invention, upon a single oral administration in a fasted state in healthy adult subjects, provides a serum AUC(inf) of about 2150±about 370 ng*h/mL. In some embodiments, a 30 mg grapiprant liquid unit dosage form of the invention, upon a single oral administration in a fasted state in healthy adult subjects, provides a serum AUC(inf) of about 2150±about 185 ng*h/mL.

In some embodiments, a 30 mg grapiprant liquid unit dosage form of the invention, upon a single oral administration in a fasted state in healthy adult subjects, provides a serum T1/2 of about 6.14±about 1.36 h. In some embodiments, a 30 mg grapiprant liquid unit dosage form of the invention, upon a single oral administration in a fasted state in healthy adult subjects, provides a serum T1/2 of about 6.14±about 0.68 h. In some embodiments, a 30 mg grapiprant liquid unit dosage form of the invention, upon a single oral administration in a fasted state in healthy adult subjects, provides a serum T1/2 of about 6.14±about 0.34 h.

In some embodiments, a 100 mg grapiprant liquid unit dosage form of the invention, upon a single oral administration in a fasted state in healthy adult subjects, provides one or more pharmacokinetics result selected from:

a mean serum Cₘₐₓ of about 1440 ng/mL;

a mean serum Tₘₐₓ of about 0.75 h;

a mean serum AUC(Tlast) of about 7170 ng*h/mL;

a mean serum AUC(inf) of about 7220 ng*h/mL; and

a mean serum T1/2 of about 6.91 h.

In some embodiments, a 100 mg grapiprant liquid unit dosage form of the invention, upon a single oral administration in a fasted state in healthy adult subjects, provides a mean serum Cₘₐₓ of about 1440 ng/mL. In some embodiments, a 100 mg grapiprant liquid unit dosage form of the invention, upon a single oral administration in a fasted state in healthy adult subjects, provides a mean serum Tₘₐₓ of about 0.75 h. In some embodiments, a 100 mg grapiprant liquid unit dosage form of the invention, upon a single oral administration in a fasted state in healthy adult subjects, provides a mean serum AUC(Tlast) of about 7170 ng*h/mL. In some embodiments, a 100 mg grapiprant liquid unit dosage form of the invention, upon a single oral administration in a fasted state in healthy adult subjects, provides a mean serum AUC(inf) of about 7220 ng*h/mL. In some embodiments, a 100 mg grapiprant liquid unit dosage form of the invention, upon a single oral administration in a fasted state in healthy adult subjects, provides a mean serum T1/2 of about 6.91 h.

In some embodiments, a 100 mg grapiprant liquid unit dosage form of the invention, upon a single oral administration in a fasted state in healthy adult subjects, provides one or more pharmacokinetics result selected from:

a serum Cₘₐₓ of about 1440±about 489 ng/mL;

a serum Tₘₐₓ of about 0.50 to about 1.00 h;

a serum AUC(Tlast) of about 7170±about 1720 ng*h/mL;

a serum AUC(inf) of about 7220±about 1720 ng*h/mL; and

a serum T1/2 of about 6.91±about 2.57 h.

In some embodiments, a 100 mg grapiprant liquid unit dosage form of the invention, upon a single oral administration in a fasted state in healthy adult subjects, provides a serum Cₘₐₓ of about 1440±about 489 ng/mL. In some embodiments, a 100 mg grapiprant liquid unit dosage form of the invention, upon a single oral administration in a fasted state in healthy adult subjects, provides a serum Cₘₐₓ of about 1440±about 244.5 ng/mL. In some embodiments, a 100 mg grapiprant liquid unit dosage form of the invention, upon a single oral administration in a fasted state in healthy adult subjects, provides a serum Cₘₐₓ of about 1440±about 122.25 ng/mL.

In some embodiments, a 100 mg grapiprant liquid unit dosage form of the invention, upon a single oral administration in a fasted state in healthy adult subjects, provides a serum Tₘₐₓ of about 0.50 to about 1.00 h. In some embodiments, a 100 mg grapiprant liquid unit dosage form of the invention, upon a single oral administration in a fasted state in healthy adult subjects, provides a serum Tₘₐₓ of about 0.625 to about 0.875 h. In some embodiments, a 100 mg grapiprant liquid unit dosage form of the invention, upon a single oral administration in a fasted state in healthy adult subjects, provides a serum Tₘₐₓ of about 0.69 to about 0.81 h.

In some embodiments, a 100 mg grapiprant liquid unit dosage form of the invention, upon a single oral administration in a fasted state in healthy adult subjects, provides a serum AUC(Tlast) of about 7170±about 1720 ng*h/mL. In some embodiments, a 100 mg grapiprant liquid unit dosage form of the invention, upon a single oral administration in a fasted state in healthy adult subjects, provides a serum AUC(Tlast) of about 7170±about 860 ng*h/mL. In some embodiments, a 100 mg grapiprant liquid unit dosage form of the invention, upon a single oral administration in a fasted state in healthy adult subjects, provides a serum AUC(Tlast) of about 7170±about 430 ng*h/mL.

In some embodiments, a 100 mg grapiprant liquid unit dosage form of the invention, upon a single oral administration in a fasted state in healthy adult subjects, provides a serum AUC(inf) of about 7220±about 1720 ng*h/mL. In some embodiments, a 100 mg grapiprant liquid unit dosage form of the invention, upon a single oral administration in a fasted state in healthy adult subjects, provides a serum AUC(inf) of about 7220±about 860 ng*h/mL. In some embodiments, a 100 mg grapiprant liquid unit dosage form of the invention, upon a single oral administration in a fasted state in healthy adult subjects, provides a serum AUC(inf) of about 7220±about 430 ng*h/mL.

In some embodiments, a 100 mg grapiprant liquid unit dosage form of the invention, upon a single oral administration in a fasted state in healthy adult subjects, provides a serum T1/2 of about 6.91±about 2.57 h. In some embodiments, a 100 mg grapiprant liquid unit dosage form of the invention, upon a single oral administration in a fasted state in healthy adult subjects, provides a serum T1/2 of about 6.91±about 1.285 h. In some embodiments, a 100 mg grapiprant liquid unit dosage form of the invention, upon a single oral administration in a fasted state in healthy adult subjects, provides a serum T1/2 of about 6.91±about 0.64 h.

In some embodiments, a 300 mg grapiprant liquid unit dosage form of the invention, upon a single oral administration in a fasted state in healthy adult subjects, provides one or more pharmacokinetics result selected from:

a mean serum Cₘₐₓ of about 12000 ng/mL;

a mean serum Tₘₐₓ of about 0.75 h;

a mean serum AUC(Tlast) of about 31200 ng*h/mL;

a mean serum AUC(inf) of about 31300 ng*h/mL; and

a mean serum T1/2 of about 7.98 h.

In some embodiments, a 300 mg grapiprant liquid unit dosage form of the invention, upon a single oral administration in a fasted state in healthy adult subjects, provides a mean serum Cₘₐₓ of about 12000 ng/mL. In some embodiments, a 300 mg grapiprant liquid unit dosage form of the invention, upon a single oral administration in a fasted state in healthy adult subjects, provides a mean serum Tₘₐₓ of about 0.75 h. In some embodiments, a 300 mg grapiprant liquid unit dosage form of the invention, upon a single oral administration in a fasted state in healthy adult subjects, provides a mean serum AUC(Tlast) of about 31200 ng*h/mL. In some embodiments, a 300 mg grapiprant liquid unit dosage form of the invention, upon a single oral administration in a fasted state in healthy adult subjects, provides a mean serum AUC(inf) of about 31300 ng*h/mL. In some embodiments, a 300 mg grapiprant liquid unit dosage form of the invention, upon a single oral administration in a fasted state in healthy adult subjects, provides a mean serum T1/2 of about 7.98 h.

In some embodiments, a 300 mg grapiprant liquid unit dosage form of the invention, upon a single oral administration in a fasted state in healthy adult subjects, provides one or more pharmacokinetics result selected from:

a serum Cₘₐₓ of about 12000±about 3240 ng/mL;

a serum Tₘₐₓ of about 0.50 to about 1.00 h;

a serum AUC(Tlast) of about 31200±about 7270 ng*h/mL;

a serum AUC(inf) of about 31300±about 7260 ng*h/mL; and

a serum T1/2 of about 7.98±about 1.73 h.

In some embodiments, a 300 mg grapiprant liquid unit dosage form of the invention, upon a single oral administration in a fasted state in healthy adult subjects, provides a serum Cₘₐₓ of about 12000±about 3240 ng/mL. In some embodiments, a 300 mg grapiprant liquid unit dosage form of the invention, upon a single oral administration in a fasted state in healthy adult subjects, provides a serum Cₘₐₓ of about 12000±about 1620 ng/mL. In some embodiments, a 300 mg grapiprant liquid unit dosage form of the invention, upon a single oral administration in a fasted state in healthy adult subjects, provides a serum Cₘₐₓ of about 12000±about 810 ng/mL.

In some embodiments, a 300 mg grapiprant liquid unit dosage form of the invention, upon a single oral administration in a fasted state in healthy adult subjects, provides a serum Tₘₐₓ of about 0.50 to about 1.00 h. In some embodiments, a 300 mg grapiprant liquid unit dosage form of the invention, upon a single oral administration in a fasted state in healthy adult subjects, provides a serum Tₘₐₓ of about 0.625 to about 0.875 h. In some embodiments, a 300 mg grapiprant liquid unit dosage form of the invention, upon a single oral administration in a fasted state in healthy adult subjects, provides a serum Tₘₐₓ of about 0.69 to about 0.81 h.

In some embodiments, a 300 mg grapiprant liquid unit dosage form of the invention, upon a single oral administration in a fasted state in healthy adult subjects, provides a serum AUC(Tlast) of about 31200±about 7270 ng*h/mL. In some embodiments, a 300 mg grapiprant liquid unit dosage form of the invention, upon a single oral administration in a fasted state in healthy adult subjects, provides a serum AUC(Tlast) of about 31200±about 3635 ng*h/mL. In some embodiments, a 300 mg grapiprant liquid unit dosage form of the invention, upon a single oral administration in a fasted state in healthy adult subjects, provides a serum AUC(Tlast) of about 31200±about 1817.5 ng*h/mL.

In some embodiments, a 300 mg grapiprant liquid unit dosage form of the invention, upon a single oral administration in a fasted state in healthy adult subjects, provides a serum AUC(inf) of about 31300±about 7260 ng*h/mL. In some embodiments, a 300 mg grapiprant liquid unit dosage form of the invention, upon a single oral administration in a fasted state in healthy adult subjects, provides a serum AUC(inf) of about 31300±about 3630 ng*h/mL. In some embodiments, a 300 mg grapiprant liquid unit dosage form of the invention, upon a single oral administration in a fasted state in healthy adult subjects, provides a serum AUC(inf) of about 31300±about 1815 ng*h/mL.

In some embodiments, a 300 mg grapiprant liquid unit dosage form of the invention, upon a single oral administration in a fasted state in healthy adult subjects, provides a serum T1/2 of about 7.98±about 1.73 h. In some embodiments, a 300 mg grapiprant liquid unit dosage form of the invention, upon a single oral administration in a fasted state in healthy adult subjects, provides a serum T1/2 of about 7.98±about 0.865 h. In some embodiments, a 300 mg grapiprant liquid unit dosage form of the invention, upon a single oral administration in a fasted state in healthy adult subjects, provides a serum T1/2 of about 7.98±about 0.433 h.

In some embodiments, a 600 mg grapiprant liquid unit dosage form of the invention, upon a single oral administration in a fasted state in healthy adult subjects, provides one or more pharmacokinetics result selected from:

a mean serum Cₘₐₓ of about 45000 ng/mL;

a mean serum Tₘₐₓ of about 0.50 h;

a mean serum AUC(Tlast) of about 92200 ng*h/mL;

a mean serum AUC(inf) of about 92500 ng*h/mL; and

a mean serum T1/2 of about 8.03 h.

In some embodiments, a 600 mg grapiprant liquid unit dosage form of the invention, upon a single oral administration in a fasted state in healthy adult subjects, provides a mean serum Cₘₐₓ of about 45000 ng/mL. In some embodiments, a 600 mg grapiprant liquid unit dosage form of the invention, upon a single oral administration in a fasted state in healthy adult subjects, provides a mean serum Tₘₐₓ of about 0.50 h. In some embodiments, a 600 mg grapiprant liquid unit dosage form of the invention, upon a single oral administration in a fasted state in healthy adult subjects, provides a mean serum AUC(Tlast) of about 92200 ng*h/mL. In some embodiments, a 600 mg grapiprant liquid unit dosage form of the invention, upon a single oral administration in a fasted state in healthy adult subjects, provides a mean serum AUC(inf) of about 92500 ng*h/mL. In some embodiments, a 600 mg grapiprant liquid unit dosage form of the invention, upon a single oral administration in a fasted state in healthy adult subjects, provides a mean serum T1/2 of about 8.03 h.

In some embodiments, a 600 mg grapiprant liquid unit dosage form of the invention, upon a single oral administration in a fasted state in healthy adult subjects, provides one or more pharmacokinetics result selected from:

a serum Cₘₐₓ of about 45000±about 11500 ng/mL;

a serum Tₘₐₓ of about 0.50 to about 1.00 h;

a serum AUC(Tlast) of about 92200±about 20700 ng*h/mL;

a serum AUC(inf) of about 92500±about 20800 ng*h/mL; and

a serum T1/2 of about 8.03±about 1.46 h.

In some embodiments, a 600 mg grapiprant liquid unit dosage form of the invention, upon a single oral administration in a fasted state in healthy adult subjects, provides a serum Cₘₐₓ of about 45000±about 11500 ng/mL. In some embodiments, a 600 mg grapiprant liquid unit dosage form of the invention, upon a single oral administration in a fasted state in healthy adult subjects, provides a serum Cₘₐₓ of about 45000±about 5750 ng/mL. In some embodiments, a 600 mg grapiprant liquid unit dosage form of the invention, upon a single oral administration in a fasted state in healthy adult subjects, provides a serum Cₘₐₓ of about 45000±about 2875 ng/mL.

In some embodiments, a 600 mg grapiprant liquid unit dosage form of the invention, upon a single oral administration in a fasted state in healthy adult subjects, provides a serum Tₘₐₓ of about 0.50 to about 1.00 h. In some embodiments, a 600 mg grapiprant liquid unit dosage form of the invention, upon a single oral administration in a fasted state in healthy adult subjects, provides a serum Tₘₐₓ of about 0.50 to about 0.75 h. In some embodiments, a 600 mg grapiprant liquid unit dosage form of the invention, upon a single oral administration in a fasted state in healthy adult subjects, provides a serum Tₘₐₓ of about 0.50 to about 0.625 h.

In some embodiments, a 600 mg grapiprant liquid unit dosage form of the invention, upon a single oral administration in a fasted state in healthy adult subjects, provides a serum AUC(Tlast) of about 92200±about 20700 ng*h/mL. In some embodiments, a 600 mg grapiprant liquid unit dosage form of the invention, upon a single oral administration in a fasted state in healthy adult subjects, provides a serum AUC(Tlast) of about 92200±about 10350 ng*h/mL. In some embodiments, a 600 mg grapiprant liquid unit dosage form of the invention, upon a single oral administration in a fasted state in healthy adult subjects, provides a serum AUC(Tlast) of about 92200±about 5175 ng*h/mL.

In some embodiments, a 600 mg grapiprant liquid unit dosage form of the invention, upon a single oral administration in a fasted state in healthy adult subjects, provides a serum AUC(inf) of about 92500±about 20800 ng*h/mL. In some embodiments, a 600 mg grapiprant liquid unit dosage form of the invention, upon a single oral administration in a fasted state in healthy adult subjects, provides a serum AUC(inf) of about 92500±about 10400 ng*h/mL. In some embodiments, a 600 mg grapiprant liquid unit dosage form of the invention, upon a single oral administration in a fasted state in healthy adult subjects, provides a serum AUC(inf) of about 92500±about 5200 ng*h/mL.

In some embodiments, a 600 mg grapiprant liquid unit dosage form of the invention, upon a single oral administration in a fasted state in healthy adult subjects, provides a serum T1/2 of about 8.03±about 1.46 h. In some embodiments, a 600 mg grapiprant liquid unit dosage form of the invention, upon a single oral administration in a fasted state in healthy adult subjects, provides a serum T1/2 of about 8.03±about 0.73 h. In some embodiments, a 600 mg grapiprant liquid unit dosage form of the invention, upon a single oral administration in a fasted state in healthy adult subjects, provides a serum T1/2 of about 8.03±about 0.365 h.

In some embodiments, a 1000 mg grapiprant liquid unit dosage form of the invention, upon a single oral administration in a fasted state in healthy adult subjects, provides one or more pharmacokinetics result selected from:

a mean serum Cₘₐₓ of about 64300 ng/mL;

a mean serum Tₘₐₓ of about 1.00 h;

a mean serum AUC(Tlast) of about 174000 ng*h/mL;

a mean serum AUC(inf) of about 175000 ng*h/mL; and

a mean serum T1/2 of about 9.41 h.

In some embodiments, a 1000 mg grapiprant liquid unit dosage form of the invention, upon a single oral administration in a fasted state in healthy adult subjects, provides a mean serum Cₘₐₓ of about 64300 ng/mL. In some embodiments, a 1000 mg grapiprant liquid unit dosage form of the invention, upon a single oral administration in a fasted state in healthy adult subjects, provides a mean serum Tₘₐₓ of about 1.00 h. In some embodiments, a 1000 mg grapiprant liquid unit dosage form of the invention, upon a single oral administration in a fasted state in healthy adult subjects, provides a mean serum AUC(Tlast) of about 174000 ng*h/mL. In some embodiments, a 1000 mg grapiprant liquid unit dosage form of the invention, upon a single oral administration in a fasted state in healthy adult subjects, provides a mean serum AUC(inf) of about 175000 ng*h/mL. In some embodiments, a 1000 mg grapiprant liquid unit dosage form of the invention, upon a single oral administration in a fasted state in healthy adult subjects, provides a mean serum T1/2 of about 9.41 h.

In some embodiments, a 1000 mg grapiprant liquid unit dosage form of the invention, upon a single oral administration in a fasted state in healthy adult subjects, provides one or more pharmacokinetics result selected from:

a serum Cₘₐₓ of about 64300±about 17100 ng/mL;

a serum Tₘₐₓ of about 0.50 to about 1.00 h;

a serum AUC(Tlast) of about 174000±about 56700 ng*h/mL;

a serum AUC(inf) of about 175000±about 56700 ng*h/mL; and

a serum T1/2 of about 9.41±about 1.49 h.

In some embodiments, a 1000 mg grapiprant liquid unit dosage form of the invention, upon a single oral administration in a fasted state in healthy adult subjects, provides a serum Cₘₐₓ of about 64300±about 17100 ng/mL. In some embodiments, a 1000 mg grapiprant liquid unit dosage form of the invention, upon a single oral administration in a fasted state in healthy adult subjects, provides a serum Cₘₐₓ of about 64300±about 8550 ng/mL. In some embodiments, a 1000 mg grapiprant liquid unit dosage form of the invention, upon a single oral administration in a fasted state in healthy adult subjects, provides a serum Cₘₐₓ of about 64300±about 4275 ng/mL.

In some embodiments, a 1000 mg grapiprant liquid unit dosage form of the invention, upon a single oral administration in a fasted state in healthy adult subjects, provides a serum Tₘₐₓ of about 0.50 to about 1.00 h. In some embodiments, a 1000 mg grapiprant liquid unit dosage form of the invention, upon a single oral administration in a fasted state in healthy adult subjects, provides a serum Tₘₐₓ of about 0.75 to about 1.00 h. In some embodiments, a 1000 mg grapiprant liquid unit dosage form of the invention, upon a single oral administration in a fasted state in healthy adult subjects, provides a serum Tₘₐₓ of about 0.875 to about 1.00 h.

In some embodiments, a 1000 mg grapiprant liquid unit dosage form of the invention, upon a single oral administration in a fasted state in healthy adult subjects, provides a serum AUC(Tlast) of about 174000±about 56700 ng*h/mL. In some embodiments, a 1000 mg grapiprant liquid unit dosage form of the invention, upon a single oral administration in a fasted state in healthy adult subjects, provides a serum AUC(Tlast) of about 174000±about 28350 ng*h/mL. In some embodiments, a 1000 mg grapiprant liquid unit dosage form of the invention, upon a single oral administration in a fasted state in healthy adult subjects, provides a serum AUC(Tlast) of about 174000±about 14175 ng*h/mL.

In some embodiments, a 1000 mg grapiprant liquid unit dosage form of the invention, upon a single oral administration in a fasted state in healthy adult subjects, provides a serum AUC(inf) of about 175000±about 56700 ng*h/mL. In some embodiments, a 1000 mg grapiprant liquid unit dosage form of the invention, upon a single oral administration in a fasted state in healthy adult subjects, provides a serum AUC(inf) of about 175000±about 28350 ng*h/mL. In some embodiments, a 1000 mg grapiprant liquid unit dosage form of the invention, upon a single oral administration in a fasted state in healthy adult subjects, provides a serum AUC(inf) of about 175000±about 14175 ng*h/mL.

In some embodiments, a 1000 mg grapiprant liquid unit dosage form of the invention, upon a single oral administration in a fasted state in healthy adult subjects, provides a serum T1/2 of about 9.41±about 1.49 h. In some embodiments, a 1000 mg grapiprant liquid unit dosage form of the invention, upon a single oral administration in a fasted state in healthy adult subjects, provides a serum T1/2 of about 9.41±about 0.745 h. In some embodiments, a 1000 mg grapiprant liquid unit dosage form of the invention, upon a single oral administration in a fasted state in healthy adult subjects, provides a serum T1/2 of about 9.41±about 0.3725 h.

In some embodiments, a 1500 mg grapiprant liquid unit dosage form of the invention, upon a single oral administration in a fasted state in healthy adult subjects, provides one or more pharmacokinetics result selected from:

a mean serum Cₘₐₓ of about 83800 ng/mL;

a mean serum Tₘₐₓ of about 0.75 h;

a mean serum AUC(Tlast) of about 242000 ng*h/mL;

a mean serum AUC(inf) of about 242000 ng*h/mL; and

a mean serum T1/2 of about 8.13 h.

In some embodiments, a 1500 mg grapiprant liquid unit dosage form of the invention, upon a single oral administration in a fasted state in healthy adult subjects, provides a mean serum Cₘₐₓ of about 83800 ng/mL. In some embodiments, a 1500 mg grapiprant liquid unit dosage form of the invention, upon a single oral administration in a fasted state in healthy adult subjects, provides a mean serum Tₘₐₓ of about 0.75 h. In some embodiments, a 1500 mg grapiprant liquid unit dosage form of the invention, upon a single oral administration in a fasted state in healthy adult subjects, provides a mean serum AUC(Tlast) of about 242000 ng*h/mL. In some embodiments, a 1500 mg grapiprant liquid unit dosage form of the invention, upon a single oral administration in a fasted state in healthy adult subjects, provides a mean serum AUC(inf) of about 242000 ng*h/mL. In some embodiments, a 1500 mg grapiprant liquid unit dosage form of the invention, upon a single oral administration in a fasted state in healthy adult subjects, provides a mean serum T1/2 of about 8.13 h.

In some embodiments, a 1500 mg grapiprant liquid unit dosage form of the invention, upon a single oral administration in a fasted state in healthy adult subjects, provides one or more pharmacokinetics result selected from:

a serum Cₘₐₓ of about 83800±about 21800 ng/mL;

a serum Tₘₐₓ of about 0.50 to about 1.00 h;

a serum AUC(Tlast) of about 242000±about 64000 ng*h/mL;

a serum AUC(inf) of about 242000±about 63900 ng*h/mL; and

a serum T1/2 of about 8.13±about 2.18 h.

In some embodiments, a 1500 mg grapiprant liquid unit dosage form of the invention, upon a single oral administration in a fasted state in healthy adult subjects, provides a serum Cₘₐₓ of about 83800±about 21800 ng/mL. In some embodiments, a 1500 mg grapiprant liquid unit dosage form of the invention, upon a single oral administration in a fasted state in healthy adult subjects, provides a serum Cₘₐₓ of about 83800±about 10900 ng/mL. In some embodiments, a 1500 mg grapiprant liquid unit dosage form of the invention, upon a single oral administration in a fasted state in healthy adult subjects, provides a serum Cₘₐₓ of about 83800±about 5450 ng/mL.

In some embodiments, a 1500 mg grapiprant liquid unit dosage form of the invention, upon a single oral administration in a fasted state in healthy adult subjects, provides a serum Tₘₐₓ of about 0.50 to about 1.00 h. In some embodiments, a 1500 mg grapiprant liquid unit dosage form of the invention, upon a single oral administration in a fasted state in healthy adult subjects, provides a serum Tₘₐₓ of about 0.625 to about 0.875 h. In some embodiments, a 1500 mg grapiprant liquid unit dosage form of the invention, upon a single oral administration in a fasted state in healthy adult subjects, provides a serum Tₘₐₓ of about 0.69 to about 0.81 h.

In some embodiments, a 1500 mg grapiprant liquid unit dosage form of the invention, upon a single oral administration in a fasted state in healthy adult subjects, provides a serum AUC(Tlast) of about 242000±about 64000 ng*h/mL. In some embodiments, a 1500 mg grapiprant liquid unit dosage form of the invention, upon a single oral administration in a fasted state in healthy adult subjects, provides a serum AUC(Tlast) of about 242000±about 32000 ng*h/mL. In some embodiments, a 1500 mg grapiprant liquid unit dosage form of the invention, upon a single oral administration in a fasted state in healthy adult subjects, provides a serum AUC(Tlast) of about 242000±about 16000 ng*h/mL.

In some embodiments, a 1500 mg grapiprant liquid unit dosage form of the invention, upon a single oral administration in a fasted state in healthy adult subjects, provides a serum AUC(inf) of about 242000±about 63900 ng*h/mL. In some embodiments, a 1500 mg grapiprant liquid unit dosage form of the invention, upon a single oral administration in a fasted state in healthy adult subjects, provides a serum AUC(inf) of about 242000±about 31950 ng*h/mL. In some embodiments, a 1500 mg grapiprant liquid unit dosage form of the invention, upon a single oral administration in a fasted state in healthy adult subjects, provides a serum AUC(inf) of about 242000±about 15975 ng*h/mL.

In some embodiments, a 1500 mg grapiprant liquid unit dosage form of the invention, upon a single oral administration in a fasted state in healthy adult subjects, provides a serum T1/2 of about 8.13±about 2.18 h. In some embodiments, a 1500 mg grapiprant liquid unit dosage form of the invention, upon a single oral administration in a fasted state in healthy adult subjects, provides a serum T1/2 of about 8.13±about 1.09 h. In some embodiments, a 1500 mg grapiprant liquid unit dosage form of the invention, upon a single oral administration in a fasted state in healthy adult subjects, provides a serum T1/2 of about 8.13±about 0.545 h.

In some embodiments, a 2000 mg grapiprant liquid unit dosage form of the invention, upon a single oral administration in a fasted state in healthy adult subjects, provides one or more pharmacokinetics result selected from:

a mean serum Cₘₐₓ of about 134000 ng/mL;

a mean serum Tₘₐₓ of about 1.00 h;

a mean serum AUC(Tlast) of about 478000 ng*h/mL;

a mean serum AUC(inf) of about 479000 ng*h/mL; and

a mean serum T1/2 of about 8.25 h.

In some embodiments, a 2000 mg grapiprant liquid unit dosage form of the invention, upon a single oral administration in a fasted state in healthy adult subjects, provides a mean serum Cₘₐₓ of about 134000 ng/mL. In some embodiments, a 2000 mg grapiprant liquid unit dosage form of the invention, upon a single oral administration in a fasted state in healthy adult subjects, provides a mean serum Tₘₐₓ of about 1.00 h. In some embodiments, a 2000 mg grapiprant liquid unit dosage form of the invention, upon a single oral administration in a fasted state in healthy adult subjects, provides a mean serum AUC(Tlast) of about 478000 ng*h/mL. In some embodiments, a 2000 mg grapiprant liquid unit dosage form of the invention, upon a single oral administration in a fasted state in healthy adult subjects, provides a mean serum AUC(inf) of about 479000 ng*h/mL. In some embodiments, a 2000 mg grapiprant liquid unit dosage form of the invention, upon a single oral administration in a fasted state in healthy adult subjects, provides a mean serum T1/2 of about 8.25 h.

In some embodiments, a 2000 mg grapiprant liquid unit dosage form of the invention, upon a single oral administration in a fasted state in healthy adult subjects, provides one or more pharmacokinetics result selected from:

a serum Cₘₐₓ of about 134000±about 51600 ng/mL;

a serum Tₘₐₓ of about 0.50 to about 1.00 h;

a serum AUC(Tlast) of about 478000±about 228000 ng*h/mL;

a serum AUC(inf) of about 479000±about 229000 ng*h/mL; and

a serum T_{1/2} of about 8.25±about 1.38 h.

In some embodiments, a 2000 mg grapiprant liquid unit dosage form of the invention, upon a single oral administration in a fasted state in healthy adult subjects, provides a serum Cₘₐₓ of about 134000±about 51600 ng/mL. In some embodiments, a 2000 mg grapiprant liquid unit dosage form of the invention, upon a single oral administration in a fasted state in healthy adult subjects, provides a serum Cₘₐₓ of about 134000±about 25800 ng/mL. In some embodiments, a 2000 mg grapiprant liquid unit dosage form of the invention, upon a single oral administration in a fasted state in healthy adult subjects, provides a serum Cₘₐₓ of about 134000±about 12900 ng/mL.

In some embodiments, a 2000 mg grapiprant liquid unit dosage form of the invention, upon a single oral administration in a fasted state in healthy adult subjects, provides a serum Tₘₐₓ of about 0.50 to about 1.00 h. In some embodiments, a 2000 mg grapiprant liquid unit dosage form of the invention, upon a single oral administration in a fasted state in healthy adult subjects, provides a serum Tₘₐₓ of about 0.75 to about 1.00 h. In some embodiments, a 2000 mg grapiprant liquid unit dosage form of the invention, upon a single oral administration in a fasted state in healthy adult subjects, provides a serum Tₘₐₓ of about 0.875 to about 1.00 h.

In some embodiments, a 2000 mg grapiprant liquid unit dosage form of the invention, upon a single oral administration in a fasted state in healthy adult subjects, provides a serum AUC(Tlast) of about 478000±about 228000 ng*h/mL. In some embodiments, a 2000 mg grapiprant liquid unit dosage form of the invention, upon a single oral administration in a fasted state in healthy adult subjects, provides a serum AUC(Tlast) of about 478000±about 114000 ng*h/mL. In some embodiments, a 2000 mg grapiprant liquid unit dosage form of the invention, upon a single oral administration in a fasted state in healthy adult subjects, provides a serum AUC(Tlast) of about 478000±about 57000 ng*h/mL.

In some embodiments, a 2000 mg grapiprant liquid unit dosage form of the invention, upon a single oral administration in a fasted state in healthy adult subjects, provides a serum AUC(inf) of about 479000±about 229000 ng*h/mL. In some embodiments, a 2000 mg grapiprant liquid unit dosage form of the invention, upon a single oral administration in a fasted state in healthy adult subjects, provides a serum AUC(inf) of about 479000±about 114500 ng*h/mL. In some embodiments, a 2000 mg grapiprant liquid unit dosage form of the invention, upon a single oral administration in a fasted state in healthy adult subjects, provides a serum AUC(inf) of about 479000±about 57250 ng*h/mL.

In some embodiments, a 2000 mg grapiprant liquid unit dosage form of the invention, upon a single oral administration in a fasted state in healthy adult subjects, provides a serum T1/2 of about 8.25±about 1.38 h. In some embodiments, a 2000 mg grapiprant liquid unit dosage form of the invention, upon a single oral administration in a fasted state in healthy adult subjects, provides a serum T1/2 of about 8.25±about 0.69 h. In some embodiments, a 2000 mg grapiprant liquid unit dosage form of the invention, upon a single oral administration in a fasted state in healthy adult subjects, provides a serum T1/2 of about 8.25±about 0.345 h.

In some embodiments, a grapiprant unit dosage form OPC of the invention comprises grapirprant, or a pharmaceutically acceptable salt thereof, and one or more pharmaceutically acceptable excipient or carrier. In some embodiments, a grapiprant liquid unit dosage form of the invention comprises grapirprant, or a pharmaceutically acceptable salt thereof, one or more pharmaceutically acceptable excipient or carrier, and water. In some embodiments, one or more pharmaceutically acceptable excipient or carrier in a grapiprant unit dosage form OPC and/or a grapiprant liquid unit dosage form of the invention is as described herein.

In some embodiments, one or more pharmaceutically acceptable excipient or carrier in a grapiprant unit dosage form OPC and/or a grapiprant liquid unit dosage form of the invention comprises hydroxypropyl cellulose. In some embodiments, a grapiprant unit dosage form OPC and/or a grapiprant liquid unit dosage form of the invention comprises about 100-300 mg, 125-275 mg, 150-250 mg, or 175-225 mg hydroxypropyl cellulose. In some embodiments, a grapiprant unit dosage form OPC and/or a grapiprant liquid unit dosage form of the invention comprises about 200 mg hydroxypropyl cellulose.

In some embodiments, one or more pharmaceutically acceptable excipient or carrier in a grapiprant unit dosage form OPC and/or a grapiprant liquid unit dosage form of the invention comprises microcrystalline cellulose. In some embodiments, a grapiprant unit dosage form OPC and/or a grapiprant liquid unit dosage form of the invention comprises about 150-450 mg, 175-425 mg, 200-400 mg, 225-375 mg, 250-350 mg, or 275-325 mg microcrystalline cellulose. In some embodiments, a grapiprant unit dosage form OPC and/or a grapiprant liquid unit dosage form of the invention comprises about 300 mg microcrystalline cellulose.

In some embodiments, one or more pharmaceutically acceptable excipient or carrier in a grapiprant unit dosage form OPC and/or a grapiprant liquid unit dosage form of the invention comprises titanium dioxide. In some embodiments, a grapiprant unit dosage form OPC and/or a grapiprant liquid unit dosage form of the invention comprises about 25-75 mg, 31.25-68.75 mg, 37.5-62.5 mg, or 43.75-56.25 mg titanium dioxide. In some embodiments, a grapiprant unit dosage form OPC and/or a grapiprant liquid unit dosage form of the invention comprises about 50 mg titanium dioxide.

In some embodiments, one or more pharmaceutically acceptable excipient or carrier in a grapiprant unit dosage form OPC and/or a grapiprant liquid unit dosage form of the invention comprises xylitol. In some embodiments, a grapiprant unit dosage form OPC and/or a grapiprant liquid unit dosage form of the invention comprises about 200-600 mg, 250-550 mg, 300-500 mg, or 350-450 mg xylitol. In some embodiments, a grapiprant unit dosage form OPC and/or a grapiprant liquid unit dosage form of the invention comprises about 400 mg xylitol.

In some embodiments, one or more pharmaceutically acceptable excipient or carrier in a grapiprant unit dosage form OPC and/or a grapiprant liquid unit dosage form of the invention comprises simethicone. In some embodiments, simethicone is a simethicone emulsion. In some embodiments, simethicone is a 30% simethicone emulsion. In some embodiments, a grapiprant unit dosage form OPC and/or a grapiprant liquid unit dosage form of the invention comprises about 16.65-49.95 mg, 20.81-45.79 mg, 24.97-41.63 mg, or 29.13-37.47 mg 30% simethicone emulsion. In some embodiments, a grapiprant unit dosage form OPC and/or a grapiprant liquid unit dosage form of the invention comprises about 33.3 mg 30% simethicone emulsion.

In some embodiments, one or more pharmaceutically acceptable excipient or carrier in a grapiprant unit dosage form OPC and/or a grapiprant liquid unit dosage form of the invention comprises about 200 mg hydroxypropyl cellulose, about 300 mg microcrystalline cellulose, about 50 mg titanium dioxide, about 400 mg xylitol, and about 33.3 mg 30% simethicone emulsion.

In some embodiments, the present invention provides a unit dosage form of a pharmaceutical composition, in liquid form, comprising about 1 mg grapiprant, or a pharmaceutically acceptable salt thereof, and about 100 mL water.

In some embodiments, the present invention provides a unit dosage form of a pharmaceutical composition in liquid form comprising, comprising essentially of, or consisting of, about 3 mg grapiprant, or a pharmaceutically acceptable salt thereof, and about 100 mL water.

In some embodiments, the present invention provides a unit dosage form of a pharmaceutical composition in liquid form comprising, comprising essentially of, or consisting of, about 10 mg grapiprant, or a pharmaceutically acceptable salt thereof, and about 100 mL water.

In some embodiments, the present invention provides a unit dosage form of a pharmaceutical composition in liquid form comprising, comprising essentially of, or consisting of, about 30 mg grapiprant, or a pharmaceutically acceptable salt thereof, about 200 mg hydroxypropyl cellulose, about 300 mg microcrystalline cellulose, about 50 mg titanium dioxide, about 400 mg xylitol, about 33.3 mg 30% simethicone emulsion, and about 100 mL water.

In some embodiments, the present invention provides a unit dosage form of a pharmaceutical composition in liquid form comprising, comprising essentially of, or consisting of, about 60 mg grapiprant, or a pharmaceutically acceptable salt thereof, about 200 mg hydroxypropyl cellulose, about 300 mg microcrystalline cellulose, about 50 mg titanium dioxide, about 400 mg xylitol, about 33.3 mg 30% simethicone emulsion, and about 100 mL water.

In some embodiments, the present invention provides a unit dosage form of a pharmaceutical composition in liquid form comprising, comprising essentially of, or consisting of, about 100 mg grapiprant, or a pharmaceutically acceptable salt thereof, about 200 mg hydroxypropyl cellulose, about 300 mg microcrystalline cellulose, about 50 mg titanium dioxide, about 400 mg xylitol, about 33.3 mg 30% simethicone emulsion, and about 100 mL water.

In some embodiments, the present invention provides a unit dosage form of a pharmaceutical composition in liquid form comprising, comprising essentially of, or consisting of, about 200 mg grapiprant, or a pharmaceutically acceptable salt thereof, about 200 mg hydroxypropyl cellulose, about 300 mg microcrystalline cellulose, about 50 mg titanium dioxide, about 400 mg xylitol, about 33.3 mg 30% simethicone emulsion, and about 100 mL water.

In some embodiments, the present invention provides a unit dosage form of a pharmaceutical composition in liquid form comprising, comprising essentially of, or consisting of, about 300 mg grapiprant, or a pharmaceutically acceptable salt thereof, about 200 mg hydroxypropyl cellulose, about 300 mg microcrystalline cellulose, about 50 mg titanium dioxide, about 400 mg xylitol, about 33.3 mg 30% simethicone emulsion, and about 100 mL water.

In some embodiments, the present invention provides a unit dosage form of a pharmaceutical composition in liquid form comprising, comprising essentially of, or consisting of, about 600 mg grapiprant, or a pharmaceutically acceptable salt thereof, about 200 mg hydroxypropyl cellulose, about 300 mg microcrystalline cellulose, about 50 mg titanium dioxide, about 400 mg xylitol, about 33.3 mg 30% simethicone emulsion, and about 100 mL water.

In some embodiments, the present invention provides a unit dosage form of a pharmaceutical composition in liquid form comprising, comprising essentially of, or consisting of, about 1000 mg grapiprant, or a pharmaceutically acceptable salt thereof, about 200 mg hydroxypropyl cellulose, about 300 mg microcrystalline cellulose, about 50 mg titanium dioxide, about 400 mg xylitol, about 33.3 mg 30% simethicone emulsion, and about 100 mL water.

In some embodiments, the present invention provides a unit dosage form of a pharmaceutical composition in liquid form comprising, comprising essentially of, or consisting of, about 1500 mg grapiprant, or a pharmaceutically acceptable salt thereof, about 200 mg hydroxypropyl cellulose, about 300 mg microcrystalline cellulose, about 50 mg titanium dioxide, about 400 mg xylitol, about 33.3 mg 30% simethicone emulsion, and about 100 mL water.

In some embodiments, the present invention provides a unit dosage form of a pharmaceutical composition in liquid form comprising, comprising essentially of, or consisting of, about 2000 mg grapiprant, or a pharmaceutically acceptable salt thereof, about 200 mg hydroxypropyl cellulose, about 300 mg microcrystalline cellulose, about 50 mg titanium dioxide, about 400 mg xylitol, about 33.3 mg 30% simethicone emulsion, and about 100 mL water.

In some embodiments, a grapiprant unit dosage form OPC of the invention comprises essentially grapirprant, or a pharmaceutically acceptable salt thereof, at an amount as described herein. In some embodiments, a grapiprant unit dosage form OPC of the invention consists of grapirprant, or a pharmaceutically acceptable salt thereof, at an amount as described herein. In some embodiments, a grapiprant liquid unit dosage form of the invention comprises essentially grapirprant, or a pharmaceutically acceptable salt thereof, and water, wherein each of grapirprant, or a pharmaceutically acceptable salt thereof, and water is at an amount as described herein. In some embodiments, a grapiprant liquid unit dosage form of the invention consists of grapirprant, or a pharmaceutically acceptable salt thereof, and water, wherein each of grapirprant, or a pharmaceutically acceptable salt thereof, and water is at an amount as described herein.

### 6. Administration and Uses

According to another aspect, the invention provides a method for inhibiting EP4, or a mutant thereof, in a biological sample or in a patient, comprising administering a unit dosage form of the invention. In some embodiments, the invention provides a method for treating an EP4-mediated disease or disorder in a patient, comprising administering a unit dosage form of the invention. In some embodiments, a unit dosage form of the invention is administered vial oral administration.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, the terms “EP4-mediated” disorders, diseases, and/or conditions as used herein means any disease or other deleterious condition in which EP4, or a mutant thereof, is known to play a role, including, but is not limited to, a cellular proliferative disorder. In some embodiments, a cellular proliferative disorder is a cancer as described herein.

The term “patient,” as used herein, means an animal, preferably a mammal, and most preferably a human.

In some embodiments, a unit dosage form of the invention is administered in a single composition as a single dosage form.

6.1. Co-Administration with One or More Other Therapeutic Agent

Depending upon the particular condition, or disease, to be treated, additional therapeutic agents that are normally administered to treat that condition, may be administered in combination with a unit dosage form of the invention. As used herein, additional therapeutic agents that are normally administered to treat a particular disease, or condition, are known as “appropriate for the disease, or condition, being treated.”

In some embodiments, the present invention provides a method of treating a disclosed disease or condition comprising administering to a patient in need thereof a unit dosage form of the invention, and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

A unit dosage form of the invention may also be used in combination with known therapeutic processes, for example, the administration of hormones or radiation.

A unit dosage form of the invention can be administered alone or in combination with one or more other therapeutic compounds, possible combination therapy taking the form of fixed combinations or the administration of a unit dosage form of the invention and one or more other therapeutic compounds being staggered or given independently of one another, or the combined administration of fixed combinations and one or more other therapeutic compounds. A unit dosage form of the invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

One or more other therapeutic agent may be administered separately from a unit dosage form of the invention, as part of a multiple dosage regimen. Alternatively, one or more other therapeutic agents may be part of a single dosage form, mixed together with a unit dosage form of the invention in a single composition. If administered as a multiple dosage regime, one or more other therapeutic agent and a unit dosage form of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a unit dosage form of the invention are administered as a multiple dosage regimen within greater than 24 hours apart.

As used herein, the term “combination,” “combined,” and related terms refers to the simultaneous or sequential administration of therapeutic agents in accordance with this invention. For example, a unit dosage form of the invention may be administered with one or more other therapeutic agent simultaneously or sequentially in separate unit dosage forms or together in a single unit dosage form. Accordingly, the present invention provides a single unit dosage form comprising an active ingredient of the invention, one or more other therapeutic agent, and a pharmaceutically acceptable carrier, adjuvant, or vehicle.

The amount of a unit dosage form of the invention and one or more other therapeutic agent (in those compositions which comprise an additional therapeutic agent as described above) that may be combined with the carrier materials to produce a single dosage form will vary depending upon the host treated and the particular mode of administration.

In those compositions which comprise one or more other therapeutic agent, the one or more other therapeutic agent and the active ingredient of the invention may act synergistically. Therefore, the amount of the one or more other therapeutic agent in such compositions may be less than that required in a monotherapy utilizing only that therapeutic agent. In such compositions a dosage of between 0.01-1,000 μg/kg body weight/day of the one or more other therapeutic agent can be administered.

The amount of one or more other therapeutic agent present in the unit dosage form of this invention may be no more than the amount that would normally be administered in a composition comprising that therapeutic agent as the only active agent. Preferably the amount of one or more other therapeutic agent in the unit dosage form of the invention will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is approved for dosing per the FDA label insert.

A unit dosage form of this invention may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a unit dosage form of this invention are another embodiment of the present invention.

### 6.1.1. Exemplary Other Therapeutic Agents

In some embodiments, one or more other therapeutic agent is a Poly ADP ribose polymerase (PARP) inhibitor. In some embodiments, a PARP inhibitor is selected from olaparib (Lynparza®, AstraZeneca); rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®, Pfizer); ribociclib (Kisqali®, Novartis); abemaciclib (Ly2835219, Eli Lilly); and trilaciclib (G1T28, G1 Therapeutics).

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In some embodiments, one or more other therapeutic agent is a platinum-based therapeutic, also referred to as platins. Platins cause cross-linking of DNA, such that they inhibit DNA repair and/or DNA synthesis, mostly in rapidly reproducing cells, such as cancer cells. In some embodiments, a platinum-based therapeutic is selected from cisplatin (Platinol®, Bristol-Myers Squibb); carboplatin (Paraplatin®, Bristol-Myers Squibb; also, Teva; Pfizer); oxaliplatin (Eloxitin® Sanofi-Aventis); nedaplatin (Aqupla®, Shionogi), picoplatin (Poniard Pharmaceuticals); and satraplatin (JM-216, Agennix).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Envinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is a proteasome inhibitor. Approved proteasome inhibitors useful in the present invention include bortezomib (Velcade®, Takeda); carfilzomib (Kyprolis®, Amgen); and ixazomib (Ninlaro®, Takeda).

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femora®, Novartis).

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzalex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (Xtandi Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFβ). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGFβ trap compound (NCT02699515); and (NCT02517398). M7824 is comprised of a fully human IgG1 antibody against PD-L1 fused to the extracellular domain of human TGF-beta receptor II, which functions as a TGFβ “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd6244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™ Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™) daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, C1-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, Z STK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, Z STK-474, buparlisib, pictrelisib, PF-4691502, BYL-719, dactolisib, XL-147, XL-765, and idelalisib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; Zd6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™)

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g. Patents International (e.g. IMS World Publications).

### 6.1.2. Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a unit dosage form of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+ (Th17) and CD8+ (Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams ET. AL., “Big opportunities for small molecules in immuno-oncology,” Cancer Therapy 2015, Vol. 14, pages 603-622, the content of which is incorporated herein by reference in its entirety. In some embodiment, an immuno-oncology agent is selected from the examples described in Table 1 of Jerry L. Adams ET. AL. In some embodiments, an immuno-oncology agent is a small molecule targeting an immuno-oncoloby target selected from those listed in Table 2 of Jerry L. Adams ET. AL. In some embodiments, an immuno-oncology agent is a small molecule agent selected from those listed in Table 2 of Jerry L. Adams ET. AL.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺(αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### 6.2. Uses

In some embodiments, the present invention provides a method for treating a cellular proliferative disorder in a patient comprising administering to the patient a unit dosage form of the invention. In some embodiments, a cellular proliferative disorder is a cancer as described herein.

In some embodiments, the cancer is small cell lung cancer, non-small cell lung cancer, colorectal cancer, breast cancer, gastric cancer, multiple myeloma, acute myeloid leukemia (AML), acute lymphoblastic leukemia (ALL), pancreatic cancer, liver cancer, hepatocellular cancer, neuroblastoma, other solid tumors or other hematological cancers.

### NSCLC Patients

In some embodiments, a cancer patient is a NSCLC patient. In some embodiments, a NSCLC patient has been treated by immunotherapy. In some embodiments, a NSCLC patient has been treated by PD-1/L1 immunotherapy. In some embodiments, a NSCLC patient has been treated by PD-1/L1 immunotherapy for a minimum of 12 weeks. In some embodiments, a NSCLC patient has progressed on PD-1/L1 immunotherapy given for a minimum of 12 weeks (aka post-PD-1/L1 NSCLC adenocarcinoma patients).

In some embodiments, a NSCLC patient has pathologically diagnosed adenocarcinoma histology of NSCLC.

In some embodiments, a NSCLC patient is an advanced (stage IIIb) and metastatic (stage IV) patient who has progressed clinically and/or radiographically per RECIST 1.1 (Response Evaluation Criteria in Solid Tumors).

In some embodiments, a NSCLC patient is at least 18 years old.

In some embodiments, a NSCLC patient has known PD-L1 positive status (>1%). In some embodiments, a NSCLC patient has a measurable disease as per RECIST 1.1. In some embodiments, a NSCLC patient has progression from a prior immunotherapy treatment with a PD-1 or PD-L1 antagonist given for a minimum of 12 weeks. In some embodiments, a prior immunotherapy may have been given with or without chemotherapy and may have been used in any line. In some embodiments, a NSCLC patient has one additional line of intervening chemotherapy following progression.

In some embodiments, a NSCLC patient has performance status of ECOG 0-1. In some embodiments, a NSCLC patient has ECOG performance status grade 0. In some embodiments, a NSCLC patient has ECOG performance status grade 1. ECOG performance status is discussed in Oken M, Creech R, Tormey D, et al. “Toxicity and response criteria of the Eastern Cooperative Oncology Group” Am J Clin Oncol. 1982; 5:649-655. ECOG performance status grade 0 refers to patients who are fully active, and are able to carry on all pre-disease performance without restriction. ECOG performance status grade 1 refers to patients who are restricted in physically strenuous activity but ambulatory and able to carry out work of a light or sedentary nature, e.g., light house work, office work.

In some embodiments, a NSCLC patient has adequate bone marrow, renal, and hepatic function as follows (within 7 days of starting therapy):
Absolute neutrophil count (ANC)≥1000/μL; and/or
Hemoglobin>9 g/dl; and/or
Platelet Count>75,000/μL; and/or
Serum creatinine≤1.5× upper limit of normal (ULN) or glomerular filtration rate (GFR)≥40 mL/min for subject with creatinine levels>1.5× institutional ULN (using the Cockcroft-Gault formula); and/or
Serum total bilirubin≤1.5×ULN or direct bilirubin≤ULN for subjects with total bilirubin levels>1.5 ULN; and/or
Aspartate aminotransferase (AST) and alanine aminotransferase (ALT)≤2.5×ULN (or ≤5× if liver metastases are present).

In some embodiments, a NSCLC patient has recovered to grade 1 or baseline for all clinically significant on-going adverse events (AEs) from prior therapy.

In some embodiments, a NSCLC patient does not have recent (within the last 12 months) history of inflammatory bowel disease (IBD), including Crohn's disease and ulcerative colitis, or non-infectious interstitial lung disease.

In some embodiments, a NSCLC patient does not have current use of nonsteroidal anti-inflammatory drugs (NSAIDs) or cyclooxygenase-2 (COX-2) inhibitors within 3 days before treatment initiation or at any time during the study unless used for management of adverse events. In some embodiments, a NSCLC patient does not use an aspirin product, or only use it at prophylactic cardiovascular doses.

In some embodiments, a NSCLC patient does not have recent (within the last 12 months) or current gastrointestinal (GI) ulcer or colitis (other than IBD) or clinically significant autoimmune disease (i.e. severe) requiring continuous systemic immunosuppressive therapy.

In some embodiments, a NSCLC patient does not have a history of severe hypersensitivity reactions to PD-1 antibodies.

In some embodiments, a NSCLC patient has not received a live vaccine within 30 days prior to the planned first dose of the instant treatment.

In some embodiments, a NSCLC patient does not have any condition requiring continuous systemic treatment with either corticosteroids (>10 mg daily prednisone equivalents) or other immunosuppressive medications within 2 weeks prior to first dose of the instant treatment. In some embodiments, a NSCLC patient has inhaled or topical steroids and physiological replacement doses of up to 10 mg daily prednisone equivalent in the absence of active autoimmune disease.

In some embodiments, a NSCLC patient does not have a known EGFR, ALK, or ROS gene alteration.

In some embodiments, a NSCLC patient has a history of smoking.

In some embodiments, a NSCLC patient does not have uncontrolled or life-threatening symptomatic concomitant disease (including known symptomatic HIV, symptomatic Hepatitis B and C, or active tuberculosis [TB]).

In some embodiments, a NSCLC patient has not received chemotherapy or an investigational agent or device, or undergone a major surgery or systemic radiation within 3 weeks of starting the instant treatment, or had inadequate healing or recovery from complications of any of these prior to starting the instant treatment.

In some embodiments, a NSCLC patient has not had potentially life-threatening second malignancy within 3 years before starting the instant treatment.

In some embodiments, a NSCLC patient does not have clinically unstable central nervous system (CNS)/brain metastasis (treated or stable CNS metastases allowed).

In some embodiments, a NSCLC patient does not have any other concurrent antineoplastic treatment except for allowed local radiation of lesions for palliation (to be considered non-target lesions after treatment).

In some embodiments, a NSCLC patient does not have clinically significant (i.e., active) cardiovascular disease, including but not being limited to:
cerebral vascular accident/stroke (<6 months prior to enrollment); and/or
myocardial infarction (<6 months prior to enrollment); and/or
unstable angina; and/or
congestive heart failure (≥New York Heart Association Classification Class II); and/or
serious cardiac arrhythmia requiring medication.

In some embodiments, a NSCLC patient does not have medical conditions requiring concomitant administration of strong CYP3A4 or P-glycoprotein inhibitors or inducers.

In some embodiments, a NSCLC patient is not pregnant or breastfeeding, or expecting to conceive children during the instant treatment.

In some embodiments, a NSCLC patient is with advanced or metastatic Post-PD-1/L1 Non-Small Cell Lung Cancer (NSCLC) adenocarcinoma.

In some embodiments, a NSCLC patient is an adult patient diagnosed with NSCLC who has been previously treated for a minimum of 12 weeks with any PD-1 or PD-L1 checkpoint inhibitor.

In some embodiments, a NSCLC patient is treated with grapiprant at a starting dose level of 300 mg twice a day (BID). In some embodiments, a NSCLC patient is treated with grapiprant and pembrolizumab for up to 2 years.

In some embodiments, a NSCLC patient is an adult patient with a histologically confirmed non-small cell lung cancer (NSCLC) adenocarcinoma.

In some embodiments, a NSCLC patient has an advanced (stage Mb) disease that is not amenable to curative intent treatment with concurrent chemoradiation and metastatic (stage IV) patients.

In some embodiments, a NSCLC patient has progressed clinically and/or radiographically per RECIST v1.1 after receiving a PD-1 or PD-L1 antagonist for a minimum of 12 weeks. In some embodiments, a NSCLC patient has received Immunotherapy with chemotherapy. In some embodiments, a NSCLC patient has received Immunotherapy without chemotherapy. In some embodiments, a NSCLC patient has received Immunotherapy in any line. In some embodiments, a NSCLC patient has received no more than one prior regimen of immunotherapy.

In some embodiments, a NSCLC patient has measurable disease per RECIST v1.1 as assessed by the local site investigator/radiology. In some embodiments, lesions situated in a previously irradiated area are considered measurable if progression has been demonstrated in such lesions.

In some embodiments, a NSCLC patient has a disease that can be safely accessed via bronchoscopic, thoracoscopic or percutaneous biopsy for multiple core biopsies (minimum of 3 passes per biopsy).

In some embodiments, a NSCLC patient has an Eastern Cooperative Oncology Group (ECOG) performance status of 0 to 1.

In some embodiments, a NSCLC patient has adequate organ function as defined in Table A below.

In some embodiments, a NSCLC patient does not use NSAIDs (eg, ibuprophen, naproxen), COX-2 inhibitors (eg, celecoxib) within 3 days before treatment initiation or at any time during the treatment. In some embodiments, a NSCLC patient uses NSAIDs (eg, ibuprophen, naproxen), COX-2 inhibitors (eg, celecoxib) within 3 days before treatment initiation or at any time during the treatment for management of A_{E}. In some embodiments, a NSCLC patient uses Aspirin products that is limited to prophylactic cardiovascular doses.

In some embodiments, a NSCLC patient does not have a known epidermal growth factor receptor (EGFR), anaplastic lymphoma kinase (ALK), or ROS gene alteration.

In some embodiments, a NSCLC patient does not have a known BRAF gene mutation.

In some embodiments, a NSCLC patient has a history of smoking (>100 cigarettes lifetime).

In some embodiments, a NSCLC patient does not have a history of severe hypersensitivity reactions to a PD-1/L1 antibody.

In some embodiments, a NSCLC patient has not received prior systemic anti-cancer therapy including investigational agents within 4 weeks prior to treatment. In some embodiments, a NSCLC patient has recovered from all AEs due to previous therapies to ≤Grade 1 or baseline. In some embodiments, a NSCLC patient has ≤Grade 2 neuropathy.

In some embodiments, a NSCLC patient has not received prior radiotherapy within 2 weeks of start of a treatment of the invention. In some embodiments, a NSCLC patient has recovered from all radiation-related toxicities, not require corticosteroids, and not have had radiation pneumonitis. In some embodiments, a NSCLC patient has a 1-week washout for palliative radiation (≤2 weeks of radiotherapy) to non-central nervous system (CNS) disease. In some embodiments, a NSCLC patient does not receive any antineoplastic treatment during a treatment of the invention, except for allowed local radiation of lesions for palliation only (to be considered non-target lesions after treatment). In some embodiments, a NSCLC patient has received a surgery, and recovered fully from the toxicity and/or complications from the intervention prior to starting a treatment of the invention.

In some embodiments, a NSCLC patient has not received a live vaccine within 30 days prior to the first dose of study treatment.

In some embodiments, a NSCLC patient has not taken strong CYP3A4 or P-glycoprotein inhibitors or inducers prior to and during a treatment of the invention. In some embodiments, a NSCLC patient has taken strong CYP3A4 or P-glycoprotein inhibitors or inducers, but transferred to other medications within ≥5 half-lives prior to dosing of a treatment of the invention.

In some embodiments, a NSCLC patient does not participate in or has not participated in a study of an investigational agent within 4 weeks prior to the first dose of a treatment of the invention. In some embodiments, a NSCLC patient has not used an investigational device within 4 weeks prior to the first dose of a treatment of the invention.

In some embodiments, a NSCLC patient does not have a diagnosis of immunodeficiency. In some embodiments, a NSCLC patient is not receiving chronic systemic steroid therapy (in dosing exceeding 10 mg daily of prednisone equivalent), or any other form of immunosuppressive therapy, within 7 days prior the first dose of a treatment of the invention.

In some embodiments, a NSCLC patient does not have a known additional potentially life-threatening malignancy that is progressing or has required active treatment within 3 years prior to the first dose of a treatment of the invention. In some embodiments, a NSCLC patient has a basal cell carcinoma of the skin. In some embodiments, a NSCLC patient has squamous cell carcinoma of the skin. In some embodiments, a NSCLC patient has carcinoma in situ (eg, breast carcinoma, cervical cancer in situ) that have undergone potentially curative therapy.

In some embodiments, a NSCLC patient does not have known active CNS metastases and/or carcinomatous meningitis (clinically stable and/or previously treated inactive CNS metastases allowed).

In some embodiments, a NSCLC patient does not have an active autoimmune disease that has required systemic treatment in past 2 years (ie, with use of disease modifying agents, corticosteroids or immunosuppressive drugs). In some embodiments, a systemic treatment is not replacement therapy (eg, thyroxine, insulin, or physiologic corticosteroid replacement therapy for adrenal or pituitary insufficiency). In some embodiments, an autoimmune disease is inflammatory bowel disease (IBD) such as Crohn's disease and ulcerative colitis.

In some embodiments, a NSCLC patient does not have a history of (non-infectious) pneumonitis that required steroids or has current pneumonitis.

In some embodiments, a NSCLC patient does not have an active infection requiring systemic therapy.

In some embodiments, a NSCLC patient does not have recent (within the last 12 months) or current GI ulcer or colitis or non-immune colitis.

In some embodiments, a NSCLC patient does not have a known history of human immunodeficiency virus (HIV) infection.

In some embodiments, a NSCLC patient does not have a known history of Hepatitis B or known active Hepatitis C virus infection.

In some embodiments, a NSCLC patient does not have clinically significant (ie, active) cardiovascular disease: cerebral vascular accident/stroke (<6 months prior to enrollment), myocardial infarction (<6 months prior to enrollment), unstable angina, congestive heart failure (≥New York Heart Association Classification Class II), or uncontrolled cardiac arrhythmia.

In some embodiments, a NSCLC patient does not have a known psychiatric or substance abuse disorder that would interfere with cooperating with a treatment of the invention.

In some embodiments, a NSCLC patient is not a woman of childbearing potential (WOCBP) who has a positive pregnancy test prior to a treatment of the invention.

In some embodiments, a NSCLC patient is not breastfeeding or expecting to conceive or father children within the projected duration of a treatment of the invention.

### CRC Patients

In some embodiments, a CRC patient is histologically confirmed advanced, metastatic, or progressive colorectal cancer (CRC). In some embodiments, microsatellite stable disease (MSS) is based on prior PCR or immunohistochemistry results.

In some embodiments, a CRC patient is at least 18 years old.

In some embodiments, a CRC patient has progressed on first line 5-FU based therapy, refused therapy or is intolerable to 5-FU based therapy.

In some embodiments, a CRC patient has a measurable disease as per RECIST 1.1 (Response Evaluation Criteria in Solid Tumors).

In some embodiments, a CRC patient has a performance status of ECOG 0-1. In some embodiments, a CRC patient has ECOG performance status grade 0. In some embodiments, a CRC patient has ECOG performance status grade 1.

In some embodiments, a CRC patient has adequate bone marrow, renal, and hepatic function as follows (within 7 days of starting therapy):
Absolute neutrophil count (ANC)≥1000/μL; and/or
Hemoglobin>9 g/dl; and/or
Platelet Count>75,000/μl; and/or
Serum creatinine≤1.5× upper limit of normal (ULN) or glomerular filtration rate (GFR)≥40 mL/min for subject with creatinine levels>1.5× institutional ULN (using the Cockcroft-Gault formula); and/or
Serum total bilirubin≤1.5×ULN or direct bilirubin≤ULN for subjects with total bilirubin levels>1.5 ULN; and/or
Aspartate aminotransferase (AST) and alanine aminotransferase (ALT)≤2.5×ULN (or ≤5× if liver metastases are present).

In some embodiments, a CRC patient has recovered to Grade 1 or baseline for all clinically significant on-going adverse events (AEs) from prior therapy.

In some embodiments, a CRC patient has completed previous treatment (including other investigational therapy) at least 3 weeks before initiation of the instant treatment.

In some embodiments, a CRC patient has not been treated with an anti-PD-1, anti-PD-L1, or anti-PD-L2 therapeutic antibody.

In some embodiments, a CRC patient has not used nonsteroidal anti-inflammatory drugs (NSAIDs) or cyclooxygenase-2 (COX-2) inhibitors within 3 days before initiation of the instant treatment, or at any time during the instant treatment, unless used for management of A_{E}. In some embodiments, a CRC patient does not use any aspirin product, or only use it at prophylactic cardiovascular doses.

In some embodiments, a CRC patient does not have a recent (within the last 12 months) history of inflammatory bowel disease (IBD), including Crohn's disease and ulcerative colitis, or non-infectious interstitial lung disease.

In some embodiments, a CRC patient does not have recent (within the last 12 months) or current gastrointestinal (GI) ulcer or colitis (other than IBD) or clinically significant autoimmune disease (i.e. severe) requiring continuous systemic immunosuppressive therapy.

In some embodiments, a CRC patient does not have any condition requiring continuous systemic treatment with either corticosteroids (>10 mg daily prednisone equivalents) or other immunosuppressive medications within 2 weeks prior to first dose of the instant treatment. In some embodiments, a CRC patient takes inhaled or topical steroids and physiological replacement doses of up to 10 mg daily prednisone equivalent in the absence of active clinically significant (severe) autoimmune disease.

In some embodiments, a CRC patient does not have a history of severe hypersensitivity reactions to chimeric or humanized antibodies.

In some embodiments, a CRC patient has not received a live vaccine within 30 days prior to the first dose of the instant treatment.

In some embodiments, a CRC patient does not receive any other concurrent antineoplastic treatment except for allowed local radiation of lesions for palliation only (to be considered non-target lesions after treatment).

In some embodiments, a CRC patient does not have uncontrolled or life-threatening symptomatic concomitant disease (including known symptomatic HIV, symptomatic Hepatitis B and C, or active tuberculosis [TB]).

In some embodiments, a CRC patient has not undergone a major surgery or systemic radiation within 3 weeks of starting the instant treatment or has inadequate healing or recovery from complications of surgery or radiation prior to starting the instant treatment.

In some embodiments, a CRC patient has not had a potentially life-threatening second malignancy within the last 3 years.

In some embodiments, a CRC patient does not have clinically unstable central nervous system (CNS)/brain metastasis (treated or stable CNS metastases allowed).

In some embodiments, a CRC patient has not had a clinically significant (i.e., active) cardiovascular disease, including but not being limited to:
cerebral vascular accident/stroke (<6 months prior to enrollment); and/or
myocardial infarction (<6 months prior to enrollment); and/or
unstable angina; and/or
congestive heart failure (≥New York Heart Association Classification Class II); and/or
serious cardiac arrhythmia requiring medication.

In some embodiments, a CRC patient does not have medical conditions requiring concomitant administration of strong CYP3A4 or P-glycoprotein inhibitors or inducers.

In some embodiments, a CRC patient is with advanced or progressive MSS CRC.

In some embodiments, a CRC patient is treated at a starting dose of Grapiprant 300 mg administered orally twice a day (BID).

In some embodiments, a CRC patient is treated with grapiprant 300 mg administered orally BID, and pembrolizumab administered 200 mg IV every 3 weeks (Q3W).

In some embodiments, a CRC patient is an adult patient with a histologically confirmed advanced, metastatic, or progressive CRC that is MSS. In some embodiments, microsatellite stability is based on prior polymerase chain reaction (PCR), Next-Gen sequencing, or immunohistochemistry results per institutional standards.

In some embodiments, a CRC patient has received at least two prior lines of therapy for advanced or metastatic CRC, at least one of which included fluorouracil. In some embodiments, a CRC patient has received adjuvant therapy, and progression occurs within 6 months of its completion.

In some embodiments, a CRC patient has measurable disease per RECIST v1.1 as assessed by the local site investigator/radiology. In some embodiments, lesions situated in a previously irradiated area are considered measurable if progression has been demonstrated in such lesions.

In some embodiments, a CRC patient has an accessible tumor that can be safely accessed for multiple core biopsies.

In some embodiments, a CRC patient has an Eastern Cooperative Oncology Group (ECOG) performance status of 0 to 1.

In some embodiments, a CRC patient has adequate organ function as defined in Table A below.

In some embodiments, a CRC patient is able to swallow and absorb oral tablets.

In some embodiments, a CRC patient is a woman who is not postmenopausal and uses contraception, or a man.

In some embodiments, a CRC patient has not received prior therapy with an anti-PD-1, anti-PD-L1, or anti-PD-L2 agent or with an agent directed to another stimulatory or co-inhibitory T-cell receptor (eg, CTLA-4, OX 40, CD137).

In some embodiments, a CRC patient does not use NSAIDs (eg, ibuprophen, naproxen), COX-2 inhibitors (eg, celecoxib) within 3 days before initiation of a treatment of the invention, or at any time during a treatment of the invention. In some embodiments, a CRC patient uses NSAIDs (eg, ibuprophen, naproxen), COX-2 inhibitors (eg, celecoxib) for management of A_{E} of a treatment of the invention. In some embodiments, a CRC patient uses an aspirin product that is limited to prophylactic cardiovascular doses.

In some embodiments, a CRC patient does not have history of severe hypersensitivity reactions to chimeric or humanized antibodies.

In some embodiments, a CRC patient has not received prior systemic anti-cancer therapy including investigational agents within 4 weeks (or 5 half-lives, whichever is shorter) prior to a treatment of the invention. In some embodiments, a CRC patient has recovered from all AEs due to previous therapies to ≤Grade 1 or baseline. In some embodiments, a CRC patient is with ≤Grade 2 neuropathy. In some embodiments, a CRC patient has received major surgery, and has fully recovered from the toxicity and/or complications from the intervention prior to starting a treatment of the invention.

In some embodiments, a CRC patient has not received prior radiotherapy within 2 weeks of start of a treatment of the invention. In some embodiments, a CRC patient has recovered from all radiation-related toxicities, does not require corticosteroids, and has not had radiation pneumonitis. In some embodiments, a CRC patient has a 1-week washout for palliative radiation (≤2 weeks of radiotherapy) to non-central nervous system (CNS) disease. In some embodiments, a CRC patient does not receive antineoplastic treatment concurrently with a treatment of the invention. In some embodiments, a CRC patient receives antineoplastic treatment for local radiation of lesions for palliation only (to be considered non-target lesions after treatment).

In some embodiments, a CRC patient has not received a live vaccine within 30 days prior to the first dose of a treatment of the invention.

In some embodiments, a CRC patient does not take strong CYP3A4 or P-glycoprotein inhibitors or inducers. In some embodiments, a CRC patient has taken strong CYP3A4 or P-glycoprotein inhibitors or inducers, but transferred to other medications within ≥5 half-lives prior to dosing of a treatment of the invention.

In some embodiments, a CRC patient does not participate in, or has not participated in, a study of an investigational agent within 4 weeks prior to the first dose of a treatment of the invention. In some embodiments, a CRC patient has not used an investigational device within 4 weeks prior to the first dose of a treatment of the invention.

In some embodiments, a CRC patient does not have a diagnosis of immunodeficiency. In some embodiments, a CRC patient does not receive chronic systemic steroid therapy (in dosing exceeding 10 mg daily of prednisone equivalent), or any other form of immunosuppressive therapy, within 7 days prior to the first dose of a treatment of the invention.

In some embodiments, a CRC patient does not have a known additional potentially life-threatening malignancy that is progressing or has required active treatment within 3 years prior to start of a treatment of the invention. In some embodiments, a CRC patient has basal cell carcinoma of the skin. In some embodiments, a CRC patient has squamous cell carcinoma of the skin. In some embodiments, a CRC patient has carcinoma in situ (eg, breast carcinoma, cervical cancer in situ) that has undergone potentially curative therapy.

In some embodiments, a CRC patient does not have known active CNS metastases and/or carcinomatous meningitis. In some embodiments, a CRC patient is with previously treated brain metastases and is radiologically stable, ie, without evidence of progression for at least 4 weeks by repeat imaging (note that the repeat imaging should be performed during study screening), and/or clinically stable and without requirement of steroid treatment for at least 14 days prior to first dose of a treatment of the invention.

In some embodiments, a CRC patient does not have an active autoimmune disease that has required systemic treatment (ie, with use of disease modifying agents, corticosteroids or immunosuppressive drugs) in 2 years prior to start of a treatment of the invention. In some embodiments, a CRC patient has received replacement therapy (eg, thyroxine, insulin, or physiologic corticosteroid replacement therapy for adrenal or pituitary insufficiency) in 2 years prior to start of a treatment of the invention. In some embodiments, an autoimmune disease includes but is not limited to inflammatory bowel disease (IBD) such as Crohn's disease and ulcerative colitis.

In some embodiments, a CRC patient does not have a history of (non-infectious) pneumonitis that required steroids or has current pneumonitis.

In some embodiments, a CRC patient does not have an active infection requiring systemic therapy.

In some embodiments, a CRC patient does not have recent (within 12 months from start of a treatment of the invention) or current GI ulcer or non-immune colitis.

In some embodiments, a CRC patient does not have a known history of human immunodeficiency virus (HIV) infection.

In some embodiments, a CRC patient does not have a known history of Hepatitis B or known active Hepatitis C virus infection.

In some embodiments, a CRC patient does not have clinically significant (ie, active) cardiovascular disease: cerebral vascular accident/stroke (<6 months prior to enrollment), myocardial infarction (<6 months prior to enrollment), unstable angina, congestive heart failure (≥New York Heart Association Classification Class II), or uncontrolled cardiac arrhythmia.

In some embodiments, a CRC patient does not have a known psychiatric or substance abuse disorder that would interfere with cooperating with a treatment of the invention.

In some embodiments, a CRC patient is not a woman of childbearing potential (WOCBP) who has a positive pregnancy test prior to a treatment of the invention.

In some embodiments, a CRC patient does not breastfeed or expect to conceive or father children within a treatment of the invention.

### Cancer

Cancer includes, in some embodiments, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, the cancer is Acute Lymphoblastic Leukemia (ALL), Acute Myeloid Leukemia (AML), Adrenocortical Carcinoma, Anal Cancer, Appendix Cancer, Atypical Teratoid/Rhabdoid Tumor, Basal Cell Carcinoma, Bile Duct Cancer, Bladder Cancer, Bone Cancer, Brain Tumor, Astrocytoma, Brain and Spinal Cord Tumor, Brain Stem Glioma, Central Nervous System Atypical Teratoid/Rhabdoid Tumor, Central Nervous System Embryonal Tumors, Breast Cancer, Bronchial Tumors, Burkitt Lymphoma, Carcinoid Tumor, Carcinoma of Unknown Primary, Central Nervous System Cancer, Cervical Cancer, Childhood Cancers, Chordoma, Chronic Lymphocytic Leukemia (CLL), Chronic Myelogenous Leukemia (CML), Chronic Myeloproliferative Disorders, Colon Cancer, Colorectal Cancer, Craniopharyngioma, Cutaneous T-Cell Lymphoma, Ductal Carcinoma In Situ (DCIS), Embryonal Tumors, Endometrial Cancer, Ependymoblastoma, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Fibrous Histiocytoma of Bone, Gallbladder Cancer, Gastric Cancer, Gastrointestinal Carcinoid Tumor, Gastrointestinal Stromal Tumors (GIST), Germ Cell Tumor, Ovarian Germ Cell Tumor, Gestational Trophoblastic Tumor, Glioma, Hairy Cell Leukemia, Head and Neck Cancer, Heart Cancer, Hepatocellular Cancer, Histiocytosis, Langerhans Cell Cancer, Hodgkin Lymphoma, Hypopharyngeal Cancer, Intraocular Melanoma, Islet Cell Tumors, Kaposi Sarcoma, Kidney Cancer, Langerhans Cell Histiocytosis, Laryngeal Cancer, Leukemia, Lip and Oral Cavity Cancer, Liver Cancer, Lobular Carcinoma In Situ (LCIS), Lung Cancer, Lymphoma, AIDS-Related Lymphoma, Macroglobulinemia, Male Breast Cancer, Medulloblastoma, Medulloepithelioma, Melanoma, Merkel Cell Carcinoma, Malignant Mesothelioma, Metastatic Squamous Neck Cancer with Occult Primary, Midline Tract Carcinoma Involving NUT Gene, Mouth Cancer, Multiple Endocrine Neoplasia Syndrome, Multiple Myeloma/Plasma Cell Neoplasm, Mycosis Fungoides, Myelodysplastic Syndrome, Myelodysplastic/Myeloproliferative Neoplasm, Chronic Myelogenous Leukemia (CML), Acute Myeloid Leukemia (AML), Myeloma, Multiple Myeloma, Chronic Myeloproliferative Disorder, Nasal Cavity Cancer, Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip Cancer, Oropharyngeal Cancer, Osteosarcoma, Ovarian Cancer, Pancreatic Cancer, Papillomatosis, Paraganglioma, Paranasal Sinus Cancer, Nasal Cavity Cancer, Parathyroid Cancer, Penile Cancer, Pharyngeal Cancer, Pheochromocytoma, Pineal Parenchymal Tumors of Intermediate Differentiation, Pineoblastoma, Pituitary Tumor, Plasma Cell Neoplasm, Pleuropulmonary Blastoma, Breast Cancer, Primary Central Nervous System (CNS) Lymphoma, Prostate Cancer, Rectal Cancer, Renal Cell Cancer, Clear cell renal cell carcinoma, Renal Pelvis Cancer, Ureter Cancer, Transitional Cell Cancer, Retinoblastoma, Rhabdomyosarcoma, Salivary Gland Cancer, Sarcoma, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Squamous Cell Carcinoma of the Head and Neck (HNSCC), Stomach Cancer, Supratentorial Primitive Neuroectodermal Tumors, T-Cell Lymphoma, Testicular Cancer, Throat Cancer, Thymoma, Thymic Carcinoma, Thyroid Cancer, Transitional Cell Cancer of the Renal Pelvis and Ureter, Triple Negative Breast Cancer (TNBC), Gestational Trophoblastic Tumor, Unknown Primary, Unusual Cancer of Childhood, Urethral Cancer, Uterine Cancer, Uterine Sarcoma, Waldenstrom Macroglobulinemia, or Wilms Tumor.

In certain embodiments, the cancer is selected from bladder cancer, breast cancer (including TNBC), cervical cancer, colorectal cancer, chronic lymphocytic leukemia (CLL), diffuse large B-cell lymphoma (DLBCL), esophageal adenocarcinoma, glioblastoma, head and neck cancer, leukemia (acute and chronic), low-grade glioma, lung cancer (including adenocarcinoma, non-small cell lung cancer, and squamous cell carcinoma), Hodgkin's lymphoma, non-Hodgkin lymphoma (NHL), melanoma, multiple myeloma (MM), ovarian cancer, pancreatic cancer, prostate cancer, renal cancer (including renal clear cell carcinoma and kidney papillary cell carcinoma), and stomach cancer.

In some embodiments, the cancer is small cell lung cancer, non-small cell lung cancer, colorectal cancer, multiple myeloma, acute myeloid leukemia (AML), acute lymphoblastic leukemia (ALL), pancreatic cancer, liver cancer, hepatocellular cancer, neuroblastoma, other solid tumors or other hematological cancers.

In some embodiments, the cancer is small cell lung cancer, non-small cell lung cancer, colorectal cancer, multiple myeloma, or AML.

The present invention further features methods and compositions for the diagnosis, prognosis and treatment of viral-associated cancers, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892)

In some embodiments, the present invention provides a method for treating a tumor in a patient in need thereof, comprising administering to the patient compound II, or a pharmaceutical salt or composition thereof, and an immuno-oncology agent as described herein. In some embodiments, the tumor comprises any of the cancers described herein. In some embodiments, the tumor comprises melanoma cancer. In some embodiments, the tumor comprises breast cancer. In some embodiments, the tumor comprises lung cancer. In some embodiments the tumor comprises small cell lung cancer (SCLC). In some embodiments, the tumor comprises non-small cell lung cancer (NSCLC).

In some embodiments, the tumor is treated by arresting further growth of the tumor. In some embodiments, the tumor is treated by reducing the size (e.g., volume or mass) of the tumor by at least 5%, 10%, 25%, 50%, 75%, 90% or 99% relative to the size of the tumor prior to treatment. In some embodiments, tumors are treated by reducing the quantity of the tumors in the patient by at least 5%, 10%, 25%, 50%, 75%, 90% or 99% relative to the quantity of tumors prior to treatment.

The compounds and compositions, according to the method of the present invention, may be administered using any amount and any route of administration effective for treating or lessening the severity of a cancer. The exact amount required will vary from subject to subject, depending on the species, age, and general condition of the subject, the severity of the disease or condition, the particular agent, its mode of administration, and the like. The compounds and compositions, according to the method of the present invention, are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disease or disorder being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound as described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The following examples are provided for illustrative purposes only and are not to be construed as limiting this invention in any manner.

### Exemplification

The compound grapiprant can be prepared by methods known to one of ordinary skill in the art, for example, as described in WO 2002/032900, WO 2005/021508, and U.S. Pat. Nos. 6,710,054 and 7,960,407, the contents of which are incorporated herein by reference in their entireties. Exemplary protocols for preparing polymorph form A of grapiprant are described in U.S. Pat. Nos. 7,960,407 and 9,265,756, the contents of which are incorporated herein by reference in their entireties.

### List of Abbreviations and Definition of Terms

A_{E} Adverse event
ALT alanine transaminase
AST aspartate transaminase
AUC Area under the curve describing change from baseline in serum drug concentration versus time
AUC(inf) AUC from time 0 to infinity
AUC(Tlast) AUC from time 0 to the last time (Tlast) with a quantifiable concentration
AUC(Tlast-inf) AUC from time Tlast to infinity
AV atrioventricular
BMI Body mass index
BP Blood pressure
bpm Beats per minute
BUN Blood urea nitrogen

### CFR Code of Federal Regulations

Clp Plasma clearance
Cmax Maximum observed serum concentration of CJ-023,423

### COSTART Coding Symbol Thesaurus of Adverse Reaction Terms

COX-1, -2 cyclooxygenase 1, cyclooxygenase 2
Cpest Estimated concentration at time Tlast
CPK Creatine phosphokinase

### CRF Case Report Form

### CRU Clinical Research Unit

### CYP Cytochrome P

### ECG Electrocardiogram

### FDA Food and Drug Administration

FID Federal identification number
FOBT Fecal occult blood test

### GCP Good Clinical Practices

GGT Gamma glutamyl transferase
hERG Ether-a-go-go-related gene
HR Heart rate (bpm)

### IRB Institutional Review Board

IUD Intrauterine device
kel apparent terminal elimination rate constant
LC/MS/MS Liquid chromatography/mass spectrometry/mass spectrometry
MTD Maximum tolerated dose
NOAEL no-observed-adverse-effect levels
NSAIDs Non-steroidal anti-inflammatory drugs

### OA Osteoarthritis

OPC Oral powder for constitution
OTC Over-the-counter, non-prescription drug
PD pharmacodynamics

### PGE2 Prostaglandin E2

### P-gp P-glycoprotein

PK pharmacokinetics
PR interval Time from beginning of the P wave to the beginning of the QRS complex.
QRS Complex Time (msec) from the beginning to the end of the QRS complex. Represents the depolarization of the ventricles.
QT interval Time (msec) from the beginning of the QRS complex to the end of the T wave and represents both ventricular depolarisation and repolarization
QTc The QT interval (msec) corrected for heart rate
QTcB QT corrected using Bazett formula: QT/(square root (60/heart rate))
QTcF QT/(cube root (60/heart rate))
RBC Red blood cell(s)
SAE Serious adverse event
SD Standard deviation
T½ Apparent terminal half-life
TESS Treatment-emergent signs and symptoms
Tmax Time of the first occurrence of Cmax
ULN Upper limits of the normal range
Vdss Volume of distribution
WBC White blood cell

### Example 1: Protocols for Preparing Polymorph Form a of Grapiprant

1.1 Protocol 1 (as Described in U.S. Pat. No. 7,960,407)

Step 1: Crude Amorphous Product

In a 4-necked round bottom flask equipped with a mechanical stirrer, thermometer, and two dropping funnels is immersed in a water bath (water bath temperature 18° C.). In the flask, to a solution of 2-[4-(2-ethyl-4,6-dimethyl-1H-imidazo[4,5-c]pyridin-1-yl)phenyl]ethanamine and triethylamine in CH₂Cl₂ is added p-tosyl isocyanate dropwise slowly from one of the dropping funnel maintaining the internal temperature below 28° C. The resulting solution is stirred at room temperature, then aqueous citric acid solution is added dropwise maintaining the internal temperature below 22° C. The resulting mixture is stirred vigorously at room temperature, then aqueous NaOH solution is added dropwise. After the completion of the addition, pH value of the solution is confirmed to be 5-5.5. The layers are then separated, and the aqueous layer is re-extracted with CH₂Cl₂ and the organic layer is combined. The organic layer is washed with the mixture of aqueous solution of citric acid and aqueous NaOH solution. After layers are separated, the aqueous layer is re-extracted with CH₂Cl₂ and the organic layer is combined. The resulting organic layer is added Na₂SO₄ and of charcoal, and the mixture is stirred gently at room temperature. After the mixture is filtered through celite pad, it is concentrated to give the crude product.

Step 2: Conversion to, and Purification of Polymorph Form A

In a round bottom, 4-necked flask equipped with a mechanical stirrer, thermometer and reflux condenser is immersed in a water bath. In the flask, hot (40° C.) acetone is added to the crude N-[({2-[4-(2-ethyl-4,6-dimethyl-1H-imidazo[4,5-c]pyridin-1-yl)phenyl]ethyl}amino) carbonyl]-4-methylbenzenesulfonamide (Step 1). The mixture is stirred at 50° C. under nitrogen atmosphere, then cooled slowly to room temperature. Acetone is added and the mixture is stirred at room temperature under nitrogen atmosphere. The crystals are filtered through paper filter, washed with acetone and dried by flowing nitrogen gas to give crystals of the title compound, which are further purified by the following procedure.

In a stainless 3-necked reactor equipped with a mechanical stirrer, thermometer and reflux condenser is immersed in a water bath. In the flask, a mixture (suspension) of the above compound in acetone is stirred at 50° C., then cooled to room temperature. Aliquot is taken out and crystals are collected by suction to prepare a sample for the HPLC analysis to determine the purity of the crystal. The mixture is stirred at room temperature under nitrogen atmosphere. The crystals are filtered off using a paper filter, washed with acetone, dried by flowing nitrogen gas and dried under reduced pressure at 40° C. The product is further purified by the following procedure.

In a round bottom, 4-necked flask equipped with a mechanical stirrer, thermometer and reflux condenser is immersed in a water bath. In the flask, acetone is added to the aforementioned crystals. The mixture is stirred at 50° C. under nitrogen atmosphere, then, cooled slowly to room temperature. Aliquot is taken out and crystals are collected by suction to prepare a sample for the HPLC analysis to determine the purity of the crystal. The mixture is stirred at room temperature under nitrogen atmosphere. The crystals are filtered through paper filter, washed with acetone, dried by flowing nitrogen gas, and dried under reduced pressure at 40° C. to give the title compound, Polymorph Form A.

1.2 Protocol 2 (as Described in U.S. Pat. No. 7,960,407)

To a clean and dry 3-neck round-bottom flask are charged 2-[4-(2-ethyl-4,6-dimethyl-1H-imidazo[4,5-c]pyridin-1-yl)phenyl]ethanamine and CH₂Cl₂. Tosyl isocyanate dissolved in CH₂Cl₂ is added to the reaction keeping the temperature below 21° C. and is stirred. The reaction is deemed complete by HPLC, and activated charcoal is added. The resulting slurry is filtered through a 0.5-micron filter into a speck free 3-neck round-bottom flask and the filter washed with CH₂Cl₂. The reaction is atmospherically concentrated to a minimum stirable volume and displacement continued with speck freed acetone until an internal temperature of 58° C. to 62° C. is achieved. The reaction is cooled to at least 30° C. and seed of N-[({2-[4-(2-Ethyl-4,6-dimethyl-1H-imidazo[4,5-c]pyridin-1-yl)phenyl]ethyl}amino)carbonyl]-4-methylbenzenesulfonamide Polymorph Form A is added. The reaction is allowed to granulate between 20° C. and 25° C. After cooling reaction to 0° C. to 5° C. and granulating, the reaction is filtered on a speck free filter. The solids are washed two times with speck free acetone cooled to 0° C. to 5° C. The wet-cake is returned to a speck free 3-neck round-bottom flask and speck free ethyl acetate is added. The slurry is heated to at least 75° C. and held for some time. The reaction is cooled to at least 30° C. and the solids are filtered on a speck free filter. The solids are washed with speck free ethyl acetate. The wet-cake is returned to the same speck free 3-neck round-bottom flask and speck free ethyl acetate is added. The slurry is heated to at least 75° C. and held for some time. The reaction is cooled to at least 30° C. and the solids are filtered on a speck free filter. The solids are washed with speck free ethyl acetate. The product is dried at 45° C. to 50° C. to yield the title product, Polymorph Form A.

The particle size generated by the above methodology generates a particle size that does not require milling. A simple hand-sieving process removes any lumps. The product is hand sieved through a speck free #25 hand sieve with 0.0278-inch openings.

1.3 Protocol 3 (as Described in U.S. Pat. No. 9,265,756)

Polymorph form A of grapiprant is prepared by slurry of Form J of grapiprant in 1:2 dichloromethane/acetone (v/v) at 25° C. Form J of grapiprant is a dichloromethane (DCM) solvate, having an unidentified amount of water. Form J crystals are prepared by precipitating grapiprant in 2:1 dichloromethane/n-heptane (2:1).

### Example 2. Manufacture of Grapiprant Tablets

### 2.1. Grapiprant 25 mg Tablet Component/Composition Table

| | | UNIT |
|---|---|---|
| | COMPENDIAL | STRENGTH |
| COMPONENT | GRADE | 25 mg/tablet |
| Grapiprant | Pharm | 25.0 ⁽¹⁾ |
| Microcrystalline cellulose | NF | 45.5 ⁽²⁾ |
| Lactose monohydrate, | NF | 22.0 |
| modified | | |
| Croscarmellose sodium | NF | 5.0 |
| Hydroxypropyl cellulose | NF | 2.0 |
| Purified water | USP | (70.0) ⁽³⁾ |
| Magnesium stearate | NF | 0.5 |
| Total | | 100.0 |

⁽¹⁾ Based on a theoretical drug substance potency of 100.0%.
⁽²⁾ The microcrystalline cellulose weight is adjusted according to slight potency changes in Grapiprant in order to maintain a constant tablet weight.
⁽³⁾ The amount of purified water required to produce an acceptable granulation may vary. Volatile, evaporated during processing.

### 2.2. Grapiprant 125 mg Tablet Component/Composition Table

| | | UNIT |
|---|---|---|
| | | STRENGTH |
| | COMPENDIAL | 125 mg |
| COMPONENT | GRADE | mg/tablet |
| Grapiprant | Pharm | 125.0 ⁽¹⁾ |
| Microcrystalline cellulose | NF | 227.5 ⁽²⁾ |
| Lactose monohydrate, | NF | 110.0 |
| modified | | |
| Croscarmellose sodium | NF | 25.0 |
| Hydroxypropyl cellulose | NF | 10.0 |
| Purified water | USP | (350.0) ⁽³⁾ |
| Magnesium stearate | NF | 2.5 |
| Total | | 500.0 |

⁽¹⁾ Based on a theoretical drug substance potency of 100.0%.
⁽²⁾ The microcrystalline cellulose weight is adjusted according to slight potency changes in Grapiprant in order to maintain a constant tablet weight.
⁽³⁾ The amount of purified water required to produce an acceptable granulation may vary. Volatile, evaporated during processing.

### 2.3. Grapiprant 25 mg Tablet Batch Formula

| COMPONENT | AMOUNT(grams) |
|---|---|
| Grapiprant | 1,512.0 ⁽¹⁾ |
| Microcrystalline cellulose | 2,718.0 ⁽²⁾ |
| Lactose monohydrate, | 1,320.0 |
| modified | |
| Croscarmellose sodium | 300.0 |
| Hydroxypropyl cellulose | 120.0 |
| Purified water | (4,200.0) ⁽³⁾ |
| Magnesium stearate | 30.0 |
| Total Weight | 6,000.0 ⁽⁴⁾ |

⁽¹⁾ Based on an actual drug substance potency of 99.1%.
⁽²⁾ The microcrystalline cellulose weight is adjusted according to slight potency changes in the Grapiprant in order to maintain a constant tablet weight
⁽³⁾ Sterile Water for Injection, USP was substituted for Purified water, USP.
⁽⁴⁾ Based on a theoretical batch size of 60,000 tablets.

### 2.4. Grapiprant 125 mg Tablet Batch Formula

| COMPONENT | AMOUNT(grams) |
|---|---|
| Grapiprant | 1,008.8 ⁽¹⁾ |
| Microcrystalline cellulose | 1,811.2 ⁽²⁾ |
| Lactose monohydrate, | 880.0 |
| modified | |
| Croscarmellose sodium | 200.0 |
| Hydroxypropyl cellulose | 80.0 |
| Purified water | (2,800.0) ⁽³⁾ |
| Magnesium stearate | 20.0 |
| Total Weight | 4,000.0 ⁽⁴⁾ |

⁽¹⁾ Based on an actual drug substance potency of 99.1%.
⁽²⁾ The microcrystalline cellulose weight is adjusted according to slight potency changes in the Grapiprant in order to maintain a constant tablet weight.
⁽³⁾ Sterile Water for Injection, USP was substituted for Purified water, USP.
⁽⁴⁾ Based on a theoretical batch size of 8,000 tablets.

### 2.5. Method of Manufacture and Packaging Procedure

### 2.5.1. Production Operations

### 2.5.1.1. Grapiprant 25 mg Tablet

1 The wet granulation was prepared in three equal parts as follows: the appropriate amounts of microcrystalline cellulose, grapiprant, croscarmellose sodium, hydroxypropyl cellulose and lactose monohydrate are added to the appropriate sized High Shear Mixer/Granulator and mixed.
2 The appropriate amount of purified water is added to the blend from step 1 while mixing for each part.
3 The granulation from step 2 is discharged from the High Shear Mixer/Granulator and added to the appropriate forced hot air oven and dried.
4 The dried granulation from each part is passed through the appropriate mill.
5 The milled granulation from step 4 is added to an appropriately sized blender and blended.
6 The appropriate amount of magnesium stearate is added to the blend from step 5 and blended.
7 A portion of the lubricated blend is subdivided for the manufacture of the grapiprant 125 mg tablets, as described below.
8 The remaining lubricated blend from step 7 is compressed into ¼″ standard round convex plain tablets using an appropriate rotary tablet press.
9 The tablets from step 8 are dedusted using an appropriate tablet deduster.

### Sampling

A representative sample of blend from step 5 is submitted to the testing and release laboratory for testing. A representative sample of tablets from step 9 is submitted to the testing and release laboratory for approval testing.

### Specification

Identity by HPLC (Example 4.1 or 4.2): Sample chromatogram exhibits a major peak with the same retention time as that of the working standard of grapiprant.

Potency by HPLC (Example 4.1 or 4.2): Average of 10 tablets is within 90.0% to 110.0% of label claim.

Purity by HPLC (Example 4.1): Each of unspecified degradation products is no greater than 0.5%.

Total degradation products by HPLC (Example 4.1): no more than 5.0%.

### 2.5.1.2. Grapiprant 125 mg Tablet

1. The lubricated blend from the grapiprant 25 mg tablets, as described above, is compressed into 13/32″ standard round convex plain tablets using an appropriate rotary tablet press.
2. The tablets from step 1 are dedusted using an appropriate tablet deduster.

### Sampling

A representative sample of tablets from step 2 is submitted to the testing and release laboratory for approval testing.

### Specification

Identity by HPLC (Example 4.1 or 4.2): Sample chromatogram exhibits a major peak with the same retention time as that of the working standard of grapiprant.

Potency by HPLC (Example 4.1 or 4.2): Average of 10 tablets is within 90.0% to 110.0% of label claim.

Purity by HPLC (Example 4.1): Each of unspecified degradation products is no greater than 0.5%.

Total degradation products by HPLC (Example 4.1): no more than 5.0%.

### 2.5.2. Packaging Procedure

The grapiprant 25 mg and 125 mg tablets are filled into approved containers. The filled containers are sealed, labeled and cartoned on automatic, semi-automatic or hand operated equipment. Filling, labeling and packaging operations are performed under standard written procedures for this product type to assure product integrity, purity and strength.

### Example 3. Manufacture of Placebo Tablets

### 3.1. Placebo for 25 mg Grapiprant Tablet Component/Composition Table

| | | UNIT |
|---|---|---|
| | | STRENGTH |
| | COMPENDIAL | 0 mg |
| COMPONENT | GRADE | mg/tablet |
| Lactose monohydrate, | NF | 76.0 |
| modified | | |
| Microcrystalline | NF | 20.0 |
| cellulose | | |
| Sodium starch glycolate | NF | 3.0 |
| Magnesium stearate | NF | 1.0 |
| Total | | 100.0 ⁽¹⁾ |

⁽¹⁾ Slight adjustment may be made in tablet weight to match the thickness of the active tablet.

### 3.2. Placebo for 125 mg Grapiprant Tablet Component/Composition Table

| | | UNIT |
|---|---|---|
| | | STRENGTH |
| | COMPENDIAL | 0 mg |
| COMPONENT | GRADE | mg/tablet |
| Lactose monohydrate, | NF | 380.0 |
| modified | | |
| Microcrystalline | NF | 100.0 |
| cellulose | | |
| Sodium starch glycolate | NF | 15.0 |
| Magnesium stearate | NF | 5.0 |
| Total | | 500.0 ⁽¹⁾ |

⁽¹⁾ Slight adjustment may be made in tablet weight to match the thickness of the active tablet.

### 3.3. Placebo for 25 mg Grapiprant Tablet Batch Formula

| COMPONENT | AMOUNT(grams) |
|---|---|
| Lactose monohydrate, | 9,120.0 |
| modified | |
| Microcrystalline cellulose | 2,400.0 |
| Sodium starch glycolate | 360.0 |
| Magnesium stearate | 120.0 |
| Total Weight | 12,000.0 ⁽¹⁾ |

⁽¹⁾ Based on a theoretical batch size of 120,000 tablets.

### 3.4. Placebo for 125 mg Grapiprant Tablet Batch Formula

| COMPONENT | AMOUNT(grams) |
|---|---|
| Lactose monohydrate, | 4,560.0 |
| modified | |
| Microcrystalline cellulose | 1,200.0 |
| Sodium starch glycolate | 180.0 |
| Magnesium stearate | 60.0 |
| Total Weight | 6,000.0 ⁽¹⁾ |

⁽¹⁾ Based on a theoretical batch size of 12,000 tablets.

### 3.5. Method of Manufacture and Packaging Procedure

### 3.5.1. Production Operations

### 3.5.1.1. Placebo for 25 mg Grapiprant Tablet

1 The lactose monohydrate, microcrystalline cellulose and the sodium starch glycolate are added to an appropriate size blender and blended.
2 The blend from step 1 is passed through the appropriate mill, added to the appropriate size blender and blended.
3 The appropriate amount of magnesium stearate is added to the blend from step 2 and blended.
4 A portion of the lubricated blend is subdivided for the manufacture of the Placebo for 125 mg grapiprant tablets, as described below.
5 The remaining lubricated blend from step 4 is compressed into ¼″ standard round convex plain tablets using an appropriate rotary tablet press.
6 The tablets from step 5 are dedusted using an appropriate tablet deduster.

### Sampling

A representative sample of tablets from step 6 is submitted to the testing and release laboratory for approval testing.

### Specification

Absence of Drug by HPLC (Example 4.1 or 4.2): Injection of an aliquot of the placebo tablet extract does not show a peak at the retention time of the grapiprant working standard.

3.5.1.2 Placebo for 125 mg grapiprant Tablet
1 The lubricated blend from the Placebo for 25 mg grapiprant tablets, as described above, is compressed into 13/32″ standard round convex plain tablets using an appropriate rotary tablet press.
2 The tablets from step 2 are dedusted using an appropriate tablet deduster.

### Sampling

A representative sample of tablets from step 2 is submitted to the testing and release laboratory for approval testing.

### Specification

Absence of Drug by HPLC (Example 4.1 or 4.2): Injection of an aliquot of the placebo tablet extract does not show a peak at the retention time of the grapiprant working standard.

### 3.5.2. Packaging Procedure

The Placebo for 25 mg and 125 mg grapiprant tablets are filled into approved containers. The filled containers are sealed, labeled and cartoned on automatic, semi-automatic or hand operated equipment. Filling, labeling and packaging operations are performed under standard written procedures for this product type to assure product integrity, purity and strength.

### Example 4. Analytical Methods

### 4.1. Identity, Assay and Purity Evaluation of Grapiprant Tablets and Tablet Granulations/Blends by Reversed-Phase Liquid Chromatography

| Column: | Inertsil Phenyl-3, 3 μm, 250 x 4.6 mm I.D. | |
|---|---|---|
| Column Temperature: | 40° | C. |
| Mobile Phase: | A: 0.2% phosphoric acid and 100 mM sodium | |
| | perchlorate in water (pH 2.1 with NaOH) | |
| | B: Acetonitrile | |
| Detection: | UV @ 210 | nm |
| Flow Rate: | 1.0 | mL/min |
| Injection Volume: | 5 | μL |
| Dissolving Solvent or | 10 mM potassium phosphate buffer, | |
| Blank: | pH 7.0/Acetonitrile: 70/30 (v/v) | |
| Assay Concentration: | 0.25 | mg/mL |
| Acquisition Time: | 55 | minutes |
| Run Time: | 65 | minutes |

Gradient Conditions for purity evaluations:

| Time (min.) | % Solvent A | % Solvent B |
|---|---|---|
| 0 | 70 | 30 |
| 3 | 70 | 30 |
| 27 | 58 | 42 |
| 39 | 20 | 80 |
| 44 | 20 | 80 |
| 46 | 70 | 30 |
| 65 | 70 | 30 |

This method has been shown to be specific and stability indicating with suitable precision and accuracy to meet its intended purpose. This method is capable of monitoring the precursors, synthetic impurities, and degradation products at the 0.05% level. Grapiprant has been shown to be quantitatively extracted from tablets and tablet granulations.

### 4.2. Identity and Assay of Grapiprant Tablets and Tablet Granulations/Blends by Reversed-Phase Liquid Chromatography

| Column: | Inertsil C8, 5 μm, 150 × 4.6 mm I.D. | |
|---|---|---|
| Column Temperature: | 40° | C. |
| Mobile Phase: | A: 0.2% phosphoric acid and 100 mM sodium | |
| 65% A:35% B | perchlorate in water (pH 2.1 with NaOH) | |
| (isocratic) | B: Acetonitrile | |
| Detection: | UV @ 265 | nm |
| Flow Rate: | 1.0 | mL/min |
| Injection Volume: | 10 | μL |
| Dissolving Solvent or | 10 mM potassium phosphate buffer, | |
| Blank: | pH 7.0/Acetonitrile: 70/30 (v/v) | |
| Assay Concentration: | 0.25 | mg/mL |
| Acquisition Time: | 10 | minutes |
| Run Time: | 65 | minutes |

This method has been shown to be specific with suitable precision and accuracy to meet its intended purpose. Grapiprant has been shown to be quantitatively extracted from tablets and tablet granulations.

### Example 5. A Phase 1, Randomized, Placebo-Controlled, Sequential Parallel Groups, Multiple Ascending Dose Study to Evaluate the Safety, Tolerability and Pharmacokinetics in Healthy Adult Subjects and Elderly Subjects with Mild Renal Impairment

Study Objectives
To evaluate the safety and tolerability of multiple ascending doses of grapiprant tablets administered for 14 days in healthy adult subjects (Cohorts 1-4).
To evaluate the pharmacokinetics of multiple ascending doses of grapiprant tablets administered for 14 days in healthy adult subjects (Cohorts 1-4).
To evaluate the safety and tolerability of grapiprant tablets administered for 14 days in elderly subjects with mild renal impairment (Cohorts 5 and 6).
To evaluate the pharmacokinetics of grapiprant tablets administered for 14 days in elderly subjects with mild renal impairment (Cohorts 5 and 6).

Methodology

Study Design: This was a randomized, investigator-blind, subject-blind, sponsor-open, placebo controlled, sequential parallel-group, multiple ascending dose study of grapiprant tablets. Eligible healthy adult subjects in 3 sequential cohorts (Cohorts 1-3) were randomized to receive grapiprant at 50 mg, 150 mg, or 300 mg BID or placebo for 14 days. Elderly subjects (≥60 years in age) with mild renal impairment were randomized in Cohorts 5 and 6 to receive grapiprant 250 mg BID or placebo for 14 days.

Diagnosis and Main Criteria for Inclusion: Cohorts 1-3 included subjects 18 to 55 years of age who were healthy, defined as no clinically relevant abnormalities identified by a detailed medical history, full physical examination, including blood pressure and heart rate measurement, 12-lead ECG and clinical laboratory tests, and had a Body Mass Index (BMI) from 18 to 30 kg/m2, inclusive; and a total body weight>50 kg (110 lbs). Cohorts 5 and 6 included elderly subjects≥60 years of age who had mild renal impairment, defined as a calculated creatinine clearance of approximately 60-80 mL/min.

Exclusion Criteria: Subjects must have met none of the following criteria to have been eligible for this study:
1 Evidence or history of clinically significant hematological, renal, endocrine, pulmonary, gastrointestinal, cardiovascular, hepatic, psychiatric, neurologic, or allergic disease (including drug allergies, but excluding untreated, asymptomatic, seasonal allergies at time of dosing)
2 Any condition possibly affecting drug absorption, eg, gastrectomy
3 Active hemorrhoidal disease
4 A positive urine drug screen
5 History of regular alcohol consumption exceeding 7 drinks/week for women or 14 drinks/week for men (1 drink=5 ounces of wine or 12 ounces of beer or 1.5 ounces of hard liquor) within 6 months of screening
6 History or evidence of habitual tobacco- or nicotine-containing products within 3 months of screening or a positive urine or blood cotinine screen
7 Treatment with an investigational drug within 30 days or 5 half-lives (whichever was longer) preceding the first dose of study medication
8 Screening 12-lead ECG demonstrating at least 1 of the following: pulse rate>100 beats per minute (bpm), QRS>120 msec, QTc>430 msec (males) and QTc>450 msec (females), or PR>220 msec
9 Pregnant or nursing women; women of childbearing potential
10 Males who were unwilling to abstain from sexual intercourse or use a condom with pregnant or lactating women for the duration of the study, including follow-up
11 Non-vasectomized males unwilling to use a condom in addition to having their female partner use another form of contraception such as an intrauterine device, diaphragm with spermicide, oral contraceptive, injectable progesterone, sub-dermal implant or a tubal ligation if the woman could have become pregnant from the time of the first dose of study medication until completion of follow-up procedures
12 Use of prescription or nonprescription drugs, vitamins, and dietary supplements within 7 days or 5 half-lives (whichever was longer) prior to the first dose of study medication. Herbal supplements must have been discontinued 30 days prior to the first dose of study medication. Excluded from this list was acetaminophen at doses of ≤2 g/day, low dose aspirin (81 mg), thyroid and hormone replacement therapies except testosterone, beta blockers such as metoprolol, calcium channel blockers such as diltiazem, and hydroxymethyl glutaryl coenzyme A (HMG-CoA) reductase inhibitors such as atorvastatin.
13 Consumption of grapefruit or grapefruit-containing products within 7 days prior to the first dose of study medication
14 Donation of blood in excess of 500 mL within 56 days prior to dosing
15 History of sensitivity to heparin or heparin-induced thrombocytopenia, if heparin was to be used to keep in-dwelling catheters patent in the study
16 History of gastrointestinal bleeding, gastric or duodenal ulcers
17 Subjects who had not been in compliance with precautions and restrictions for fecal occult blood test
18 Positive fecal occult blood at screening
19 Positive urinalysis for blood or 1+protein at screening
20 History of diarrhea within 2 months prior to dosing.

Study Treatment: Grapiprant tablets and placebo tablets are comprises of 25 mg and 125 mg tablets.

Dosing and Duration: In Cohorts 1-3, study medication was administered once daily in the morning on Days 1 and 14, and twice daily (BID, approximately every 12 hours) on Days 2 through 13. In Cohorts 5 and 6, study medication was administered once daily in the morning on Day 1, BID on Days 4 through 16, and once in the morning on Day 17. In all cohorts dosing was to be in the fasting state for the morning dose and on an “empty stomach” for the evening dose.

Pharmacokinetic Evaluations: Serial blood samples for serum PK evaluations of grapiprant began prior to dosing on Day 1 and continued until 48 hours following Day 14 dosing (Cohorts 1-3) or until 24 hours following Day 17 dosing (Cohorts 5 and 6).

Serial urine samples for PK evaluations of grapiprant were collected on Day 14 for up to 48 hours postdose (Cohorts 1-3) or on Day 17 for up to 24 hours postdose (Cohorts 5 and 6).

Serum samples were assayed for grapiprant using 2 validated analytical methods. The first method involved on-column extraction, followed by liquid chromatography/tandem mass spectrometry (LC/MS/MS). It was discovered during the study that the system was prone to contamination under this extraction method. Therefore, the assay extraction method was revised to avoid system contaminants. The revised method consisted of extraction by protein precipitation and was followed by LC/MS/MS. Samples from Cohorts 1 and 2 were analyzed by the on-column extraction method. The remaining samples (Cohorts 3, 5, and 6) were assayed by the revised protein precipitation extraction method. The dynamic range for both assays was 1.00 to 1000 ng/mL. Pharmacokinetic parameters for grapiprant were determined from serum concentration-time data using standard noncompartmental methods.

Grapiprant was extracted from the urine by protein precipitation, and then analyzed by a validated LC/MS/MS method. The dynamic range of the assay was 1.00 to 600 μg/mL. The following parameters were calculated under the assumption that the amount of grapiprant excreted unchanged in the urine over the dosing interval was at steady state on the 14th day of dosing: amount of parent drug excreted (A_{E}), percent of dose renally excreted unchanged (A_{E}/dose), renal clearance (CL_{R}), and renal clearance of unbound drug (CL_{R}/Fu).

Safety Evaluations: Safety evaluations included clinical monitoring, adverse events (AEs), clinical laboratory tests including renal function tests, bone markers (serum bone-specific alkaline phosphatase and osteocalcin and urinary N-terminal telopeptide of type I collagen), vital signs (heart rate and blood pressure), and 12-lead ECGs.

Statistical Methods:

Serum and Urine Pharmacokinetics: Data collected on serum and urine concentrations of grapiprant were tabulated and/or plotted for each subject for each treatment regimen. Data listings and summary statistics of pharmacokinetic parameters were utilized.

Safety Analyses: Safety data are presented in tabular and/or graphical format and summarized descriptively.

For Cohorts 5 and 6, renal function was assessed by BUN, serum creatinine and creatinine clearance, evaluated as change from baseline and by predefined categorical analyses. Bone markers were analyzed as percentage change from baseline and the influence of potential outliers was assessed.

Results

Subject Disposition and Demography: In each cohort 12 subjects were randomized in a 3:1 ratio to grapiprant or placebo. Subject disposition and the number of subjects analyzed are shown in Table 5-1.

TABLE 5-1
Subject Disposition and Subjects Analyzed.

| | Healthy Adults, Cohorts 1-3 | | | | Elderly, Cohorts 5-6 | |
|---|---|---|---|---|---|---|
| | 50 mg | 150 mg | 300 mg | | 250 mg | |
| | BID | BID | BID | Placebo | BID | Placebo |
| Planned | 9 | 9 | 9 | 9 | 18 | 6 |
| Assigned to | 9 | 9 | 9 | 9 | 15 | 6 |
| Treatment | | | | | | |
| Treated | 9 | 9 | 9 | 9 | 15 | 6 |
| Completed | 8 | 9 | 9 | 8 | 14 | 6 |
| Discontinued | 1 | 0 | 0 | 1 | 1 | 0 |
| Evaluated | 9^{a} | 9 | 9^{b} | ND | 15^{a} | ND |
| for PK | | | | | | |
| Analyzed | | | | | | |
| for Safety: | | | | | | |
| Adverse | 9 | 9 | 9 | 9 | 15 | 6 |
| events | | | | | | |
| Laboratory | 9 | 9 | 9 | 9 | 15 | 6 |
| data | | | | | | |

Abbreviations:
ND = not done,
PK = pharmacokinetic
^{a}One subject in each of these cohorts withdrew front the study before Day 14 and is therefore included only in the Day 1 PK analyses.
^{b}One subject in this cohort did not have sufficient data to estimate t_{1/2} on Day 14; this subject was included in all other PK analyses.

The demographic characteristics of subjects are summarized by cohort in Table 5-2.

TABLE 5-2
Subject Disposition and Subjects Analyzed.

| | Healthy Adults, Cohorts 1-3 | | | | Elderly, Cohorts 5-6 | |
|---|---|---|---|---|---|---|
| | 50 mg | 150 mg | 300 mg | | 250 mg | |
| | BID | BID | BID | Placebo | BID | Placebo |
| No. of | 9 | 9 | 9 | 9 | 15 | 6 |
| Subjects | | | | | | |
| Sex: | | | | | | |
| Male | 8 | 9 | 9 | 9 | 5 | 1 |
| Female | 1 | 0 | 0 | 0 | 10 | 5 |
| Mean Age | 34.2 | 31.1 | 30.2 | 29.3 | 69.1 | 64.8 |
| (years) | | | | | | |
| Range | 23-54 | 18-52 | 22-42 | 19-41 | 60-77 | 61-68 |
| Race: | | | | | | |
| White | 4 | 7 | 5 | 7 | 15 | 6 |
| Black | 5 | 2 | 4 | 2 | 0 | 0 |
| Mean | 79.0 | 76.8 | 83.7 | 81.2 | 69.1 | 60.4 |
| Weight | | | | | | |
| (kg) | | | | | | |
| Range | 64.5- | 66.8- | 71.7- | 63.5- | 55.1- | 48.8- |
| | 90.8 | 92.2 | 100.3 | 105.4 | 92.0 | 69.8 |
| Mean Body | 25.3 | 24.1 | 25.6 | 24.9 | 25.3 | 23.1 |
| Mass | | | | | | |
| Index | | | | | | |
| (kg/m²) | | | | | | |
| Range | 20.6- | 20.1- | 22.9- | 20.7- | 21.3- | 19.1- |
| | 29.6 | 28.3 | 28.4 | 30.7 | 29.9 | 27.3 |
| Mean | 177.0 | 178.9 | 180.6 | 180.3 | 164.9 | 161.7 |
| Height | | | | | | |
| (cm) | | | | | | |
| Range | 165.1- | 170.2- | 175.3- | 170.2- | 149.9- | 160.0- |
| | 182.9 | 190.5 | 188.0 | 190.5 | 175.3 | 167.6 |

Pharmacokinetic Results: Following 14 days of multiple dosing with grapiprant, mean systemic exposure parameters (AUC and Cmax) of grapiprant in healthy adult subjects increased in an approximately dose proportional manner between 50 and 150 mg BID. When the dose was increased from 150 mg BID to 300 mg BID, a greater than dose proportional increase in exposure was observed. A dose of 250 mg BID administered to elderly subjects with mild renal impairment (creatinine clearance: 60-80 mL/min) resulted in a mean exposure level slightly greater than that of the 300 mg BID healthy adult group. In both healthy and elderly subjects, renal clearance, corrected for protein binding, was much greater than the glomerular filtration rate (GFR), suggesting considerable active renal secretion of grapiprant. Approximately 21% to 28% of the total daily dose administered was excreted in the urine unchanged.

The serum grapiprant PK parameters are summarized in Table 5-3.

TABLE 5-3
Mean (SD) Grapiprant Serum Pharmacokinetic Parameters Following a Single
Oral Dose on Day 1 and Following 14 Days of Multiple Oral Dosing.

| | 50 mg BID | | | | 150 mg BID | | | |
|---|---|---|---|---|---|---|---|---|
| | Day 1 (n = 9) | | Day 14 (n = 8) | | Day 1 (n = 9) | | Day 14 (n =9) | |
| Cₘₐₓ (ng/mL) | 578 | (381) | 611 | (316) | 2150 | (1390) | 2670 | (1160) |
| AUC_{T} (ng*h/mL) | 3150 | (1490) | 3370 | (1420) | 8970 | (3880) | 10300 | (3350) |
| R_{ac} (Day 14/Day 1)^{a} | N/A | | 1.13 | (0.153) | N/A | | 1.20 | (0.165) |
| Tₘₐₓ (h)^{b} | 1.50 | (0.50-3.00) | 1.25 | (0.50-3.00) | 1.50 | (1.00-3.00) | 1.50 | (0.50-4.00) |

| | | | | | | |
|---|---|---|---|---|---|---|
| t_{1/2} (h) | NR^{c} | 6.47 | (1.40) | NR^{c} | 9.71 | (1.18) |

| | 250 mg BID Elderly | | | | 300 mg BID | | | |
|---|---|---|---|---|---|---|---|---|
| | Day 1 (n = 15) | | Day 17 (n = 14) | | Day 1 (n = 9) | | Day 14 (n = 9) | |
| Cₘₐₓ (ng/mL) | 10300 | (5710) | 12300 | (4020) | 8240 | (5390) | 10400 | (5000) |
| AUC_{T} (ng*h/mL) | 30100 | (12800) | 40200 | (17300) | 24900 | (10300) | 32300 | (11900) |
| R_{ac} (Day 14/Day 1)^{a} | N/A | | 1.54 | (0.841) | N/A | | 1.33 | (0.334) |
| Tₘₐₓ (h)^{b} | 1.00 | (0.50-1.50) | 1.00 | (0.50-1.50) | 1.50 | (0.50-4.00) | 1.00 | (0.50-4.00) |

| | | | | | | |
|---|---|---|---|---|---|---|
| t_{1/2} (h) | 8.63 | (2.22) | NR^{c} | NR^{c} | 8.76 | (2.35) |

^{a}R_{ac} = Day 14 or Day 17 AUC_{T}/Day 1 AUC_{T}
^{b}Median (range)
^{c}t_{1/2} not reported (NR) due to insufficient duration of PK sampling

The urine grapiprant PK parameters are summarized in Table 5-4.

TABLE 5-4
Mean(SD) Grapiprant Urine Pharmacokinetic Parameters
Following 14 Days of Multiple Oral Dosing.

| Dose | | A_{E} (mg) | A_{E}/Dose (%) | CL_{R} (mL/min) | CL_{R}/Fu (mL/mm) | |
|---|---|---|---|---|---|---|
| 50 | mg BID (n = 8) | 10.6 (3.18) | 21.2 (6.37) | 56.3 (14.5) | 6118 | (1580) |
| 150 | mg BID (n = 9) | 33.3 (11.2) | 22.2 (7.48) | 55.2 (12.1) | 6004 | (1313) |
| 300 | mg BID (n = 9) | 82.4 (27.1) | 27.5 (9.05) | 44.5 (10.0) | 4838 | (1085) |
| 250 | mg BID (Elderly) (n = 14) | 57.9 (18.7) | 23.2 (7.48) | 25.0 (4.69) | 2719 | (510) |

Safety Results: No deaths were reported in this study. One healthy subject (150 mg BID) had serious AEs of decreased hemoglobin, decreased hematocrit, and gastrointestinal hemorrhage, which were considered to be related to the study medication. One elderly subject (250 mg BID) was withdrawn due to a non-drug related A_{E} of ventricular tachycardia. Among the healthy subjects, 4 subjects receiving 50 mg BID reported 7 AEs, 7 subjects receiving 150 mg BID reported 23 AEs, 6 subjects receiving 300 mg BID reported 13 AEs, and 4 subjects receiving placebo reported 10 AEs. Among the elderly subjects, 14 subjects receiving 250 mg BID reported 44 AEs and 4 subjects receiving placebo reported 18 AEs. Most treatment-emergent AEs were mild.

Results were similar for grapiprant and placebo and there were no clinically significant findings for laboratory safety tests, including renal function tests.

For the bone markers in healthy subjects, the following results were statistically significant. The change from baseline in bone-specific alkaline phosphatase was significantly greater in subjects who received 300 mg BID than in subjects who received placebo (20.80% versus 4.70% on Day 7, p=0.020, and 14.36% versus −5.36% on Day 14, p=0.006; expressed as least squares means). The change from baseline in osteocalcin was significantly less on Day 7 in subjects who received 50 mg BID compared with subjects who received placebo (1.40% versus 39.30%, p=0.023) and on Day 14 in subjects who received 300 mg BID compared with subjects who received placebo (14.37% versus 47.84%, p=0.045). The change from baseline in urine N-telopeptide was significantly less on Day 14 in subjects who received 150 mg BID than in subjects who received placebo (−3.97% versus 34.74%, p=0.050). There were no statistically significant differences between the treatment groups for elderly subjects.

Bone markers were evaluated in this study because of the possibility of an effect of an EP4 receptor antagonist on bone metabolism. Although there were some statistically significant differences between grapiprant and placebo groups in healthy subjects, the findings are difficult to interpret because they are of small magnitude and there was a concomitant reduction in N-telopeptide, which is a marker of bone destruction.

For systolic blood pressure measured using robust triplicate BP monitoring in-house, healthy subjects who received doses up to 300 mg BID showed no difference between changes from baseline compared with placebo while elderly subjects with mild renal impairment who received 250 mg BID showed a small increase from baseline compared with the corresponding placebo group. There were no clinically significant mean changes from baseline in vital signs and ECGs, and no other apparent differences between the grapiprant and placebo groups.

Conclusions:
Multiple-dose administration of grapiprant for 14 days was well tolerated at doses up to 300 mg BID in healthy adult subjects and at 250 mg BID in elderly subjects with mild renal impairment.
Following multiple-dose administration of grapiprant as a tablet in the fasted state, mean systemic exposures of grapiprant increased with dose in an approximately dose proportional manner between 50 mg BID and 150 mg BID and demonstrated a greater than dose proportional increase when the dose was further increased to 300 mg BID.
There was some accumulation of grapiprant at steady state after BID dosing (13% to 54% across all dose groups).
When normalized for dose, exposure in elderly subjects with mild renal impairment was slightly greater than that observed in healthy adults. Protein binding-adjusted renal clearance of grapiprant was greater than the estimated population GFR, which suggests that the unchanged compound is actively secreted in the urine.

### Example 6. A Phase 1, Randomized, Open-Label, Crossover Study to Evaluate the Effect of Food on the Pharmacokinetics of Grapiprant Tablets in Healthy Adult Subjects Study Objectives

1 To evaluate the effect of food on the pharmacokinetics of a single oral dose of grapiprant tablets in healthy adult subjects.
2 To evaluate the effect of food on the safety and tolerability of a single oral dose of grapiprant tablets in healthy adult subjects.

### Study Design:

This study was a randomized, open-label, single dose, 2-period crossover design. Each subject participated in 2 study periods. In each study period, subjects received a dose of 375 mg administered as 3×125 mg tablets of grapiprant under fasted or fed conditions in a crossover manner according to a randomized dosing sequence. The fed condition consisted of a standardized high-fat meal. Following an initial screening period of no longer than 28 days, 12 eligible subjects were to be randomized to the 2 treatment sequences and 2 treatment period sessions as shown in Table 6-1.

TABLE 6-1
Study Schematic.

| | Number of | | | |
|---|---|---|---|---|
| | Subjects | Period 1 | Washout | Period 2 |
| Sequence 1 | 6 | Fed | | Fasted |
| Sequence 2 | 6 | Fasted | | Fed |

Six subjects were randomized to each treatment sequence. Each treatment session day was separated by at least a 7-day washout period. The entire study duration following Period 1 dosing was 15 days for all subjects.

### Study Population and Criteria for Inclusion:

After giving written informed consent, healthy volunteers, men or women of non-childbearing potential, between 18 and 55 years of age, inclusive were to be enrolled in the study. Subjects were required to be in good health as determined by a detailed medical history, full physical examination, including blood pressure and pulse rate measurement, 12-lead electrocardiogram (ECG) and clinical laboratory evaluations. All subjects were to weigh>50 kg (>110 lb) and have a body mass index between 18-30 kg/m2.

Exclusion Criteria:
1 Evidence or history of clinically significant hematological, renal, endocrine, pulmonary, gastrointestinal, cardiovascular, hepatic, psychiatric, neurologic, or allergic disease (including drug allergies, but excluding untreated, asymptomatic, seasonal allergies at time of dosing)
2 Any condition possibly affecting drug absorption, eg, gastrectomy
3 Active hemorrhoidal disease
4 A positive urine drug screen
5 A history of regular alcohol consumption exceeding 7 drinks/week for women or 14 drinks/week for men (1 drink=5 ounces of wine or 12 ounces of beer or 1.5 ounces of hard liquor) within 6 months of screening
6 History or evidence of habitual tobacco- or nicotine-containing products within 3 months of screening or a positive urine or blood cotinine screen
7 Treatment with an investigational drug within 30 days or 5 half-lives (whichever was longer) preceding the first dose of study medication
8 Screening 12-lead ECG demonstrating at least one of the following: pulse rate>100 bpm, QRS>120 msec, QTc>430 msec (males) and QTc>450 msec (females) or PR>220 msec
9 Pregnant or nursing women; women of childbearing potential as defined in Section 11, Item Protocol Section 3.3.4
10 Males who were unwilling to abstain from sexual intercourse or use a condom with pregnant or lactating women for the duration of the study, including follow-up
11 Non-vasectomized males unwilling to use a condom in addition to having their female partner use another form of contraception such as an intrauterine contraceptive device (IUD), diaphragm with spermicide, oral contraceptive, injectable progesterone, sub-dermal implant or a tubal ligation if the woman could become pregnant, from the time of the first dose of study medication until completion of follow-up procedures
12 Use of prescription or nonprescription drugs, vitamins and dietary supplements, within 7 days or 5 half-lives (whichever was longer) prior to the first dose of study medication. Herbal supplements and hormone replacement therapy were to be discontinued 30 days prior to the first dose of study medication. Excluded from this list was acetaminophen at doses of ≤2 g/day.
13 Consumption of grapefruit or grapefruit juice within 7 days prior to dosing
14 Donation of blood in excess of 500 mL within 56 days prior to dosing
15 A history of sensitivity to heparin or heparin-induced thrombocytopenia, if heparin was to be used to keep in-dwelling catheters patent in the study
16 A history of gastrointestinal bleeding, gastric or duodenal ulcers
17 Had not been in compliance with precautions and restrictions for fecal occult blood test (Section 11, Item 1, Protocol Section 3.6.1.4)
18 Positive fecal occult blood at Screening
19 Positive urinalysis for blood or 1+protein at Screening
20 A history of diarrhea within 2 months prior to dosing
21 A history of intolerance of dairy products
22 A history of hypersensitivity to sulfonamides

### Treatments:

In each study period, subjects received 375 mg grapiprant (as 3×125 mg tablets) under either fed or fasted conditions. Each subject received 2 single doses: Day 1 and Day 8.

### Criteria for Evaluation and Methodology:

### Safety:

All subjects were evaluated for safety, which was assessed by adverse events either spontaneously reported or elicited by non-leading questions from study staff, clinical observations, safety laboratory tests, vital signs, 12-lead ECGs, concomitant medication assessment, physical examination, and fecal occult blood tests.

### Pharmacokinetics:

Blood samples were collected at the following times: 0 (just prior to dosing), 0.5, 1, 1.5, 2, 2.5, 3, 4, 6, 8, 12, 16, 24 and 48 hours following each dosing. The primary pharmacokinetic endpoints of the study were AUC(0-tlast) (area under the serum concentration-time curve from time 0 to the time of the last quantifiable concentration), AUC(0-inf) (AUC from time 0 to infinite time), and Cmax (maximum observed serum concentration) of serum grapiprant. The secondary pharmacokinetic endpoints of the study were Tmax (the time to Cmax) and t½ (terminal elimination half-life), as the data permitted.

### Statistical Methods:

Log-transformed AUC(0-inf), AUC(0-tlast) and Cmax were each analyzed using a mixed effects model containing sequence, treatment (fed and fasted), and period as the fixed effects and subject (within sequence) as the random effect. The SAS procedure PROC MIXED was used for the analysis. Compound symmetry was assumed, and Restricted Maximum Likelihood Estimates (REML) were utilized.

For AUC(0-inf), AUC(0-tlast) and Cmax, on the natural log scale, estimates of the adjusted mean difference between grapiprant tablet under fed condition (test) and grapiprant under fasted condition (reference) and their corresponding 90% confidence intervals were calculated.

The anti-logs (exponent) of the difference and confidence interval were taken to estimate the mean ratio (relative bioavailability) and its corresponding 90% confidence interval. Vital signs, adverse events, ECG parameters, and safety laboratory values were summarized with descriptive statistics.

### Results:

Subject Disposition: Twelve (12) healthy adults (8 males and 4 females) 33 to 54 years of age, inclusive, entered and completed the study. All 12 subjects were analyzed for pharmacokinetics and safety in each period.

Safety: There were no deaths, serious adverse events, or withdrawals due to adverse events in this study. After the 375 mg tablet-fasted treatment, a total of 7 treatment-emergent adverse events were reported in 5 subjects. Three of these events reported by 2 subjects were considered treatment-related. After the 375 mg tablet-fed treatment, a total of 5 treatment-emergent adverse events were reported in 4 subjects. Two of these events reported by 1 subject were considered treatment-related. All of the adverse events reported after both treatments were mild in intensity and resolved without intervention.

No subjects had clinically significant changes from baseline in clinical laboratory tests or vital sign measurements.

Most of the mean changes from baseline in QT or QTc intervals were negative (decreased duration). No subjects had QTc values that met the protocol-specified criteria for potential clinical significance (>500 msec), and no subjects had maximum QTc increases from baseline of ≥30 msec.

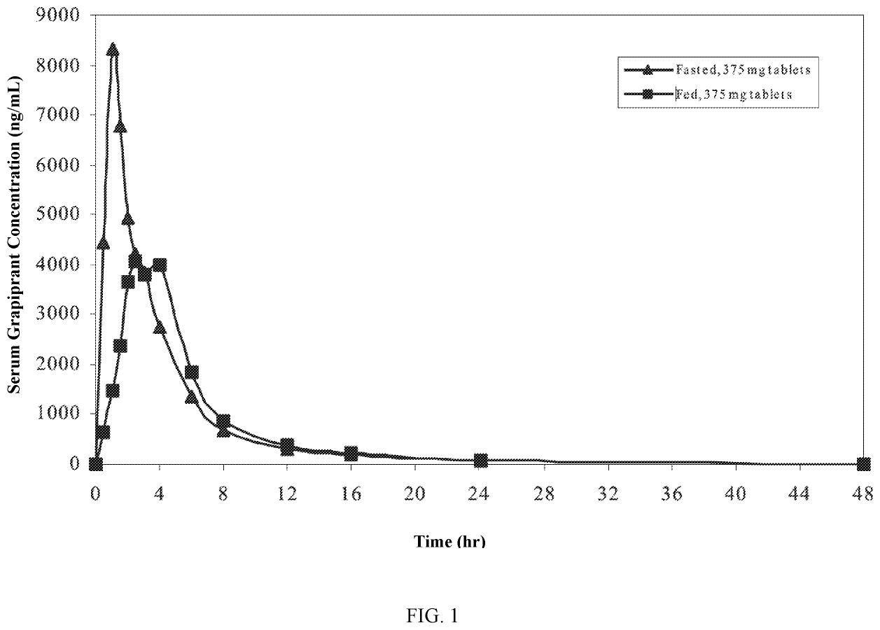

Pharmacokinetics: Compared to the fasted condition, a standard high-fat meal decreased the rate of grapiprant absorption as indicated by a 36% decrease in Cmax and a delay in Tmax (˜3 hours later). However, the extent of the absorption (as measured by AUC) was not affected by food. The serum concentration vs. time profiles of grapiprant under fasted and fed conditions are shown in FIG. 1.

The pharmacokinetic parameters of grapiprant following a single oral dose of 375 mg tablets under fasted and fed conditions are summarized in Table 6-1.

TABLE 6-1
Arithmetic Mean (SD) Pharmacokinetic Parameters of Grapiprant
Following Oral Administration of 375 mg Tablets of Grapiprant
in Healthy Subjects under Fasted and Fed Conditions (N = 12)

| | Treatment | | |
|---|---|---|---|
| Pharmacokinetic | | 375 mg tablet- | 375 mg tablet- |
| Parameter | | fasted | fed |

| | | | | |
|---|---|---|---|---|
| AUC(0-tlast) | 28900 | (13000) | 24600 | (8660) |
| (ng · h/mL) | | | | |
| AUC(0-inf) | 29100 | (13000) | 24800 | (8720) |
| (ng · h/mL) | | | | |
| Cmax (ng/mL) | 8990 | (4270) | 5840 | (2890) |
| Tmax (h)^{a} | 1 | (0.5-3) | 4 | (1.5-4) |
| t½ (h) | 9.48 | (1.87) | 8.52 | (1.29) |

AUC(0-tlast) = area under the concentration-time curve from 0 to the time of the last quantifiable concentration
AUC(0-inf) = area under the concentration-time curve from 0 to infinite time
Cmax = maximum observed concentration
Tmax = the time to Cmax
t½ = terminal elimination half-life
^{a}Median and (range) are displayed for Tₘₐₓ

Results from the statistical analyses are shown in Table 6-2. On average, Cmax and AUC decreased 36% and 13%, respectively, when grapiprant was given with a standard high-fat meal compared to the fasted condition. The absorption rate of grapiprant was decreased when administered with a high-fat meal as evidenced by the decrease in Cmax and the later Tmax compared to the fasted condition. In contrast, the 90% CI of adjusted geometric mean ratio of AUC fell within the acceptance criteria (80%, 125%), indicating that a standard high-fat meal had no effect on the extent of the absorption of grapiprant.

TABLE 6-2
Summary of Statistical Analyses Comparing the Pharmacokinetic
Parameters of 375 mg Tablets of Grapiprant in Healthy
Subjects Under Fasted and Fed Conditions (N = 12)

| Pharmacokinetic | Geometric Mean | 90% Confidence | Within-Subject |
|---|---|---|---|
| Parameter | Ratio^{a} (%) | Interval (%) | Variability |
| AUC(0-inf) | 87.48 | (81.37, 94.04) | 9.78% |
| (ng · h/mL) | | | |
| AUC(0-tlast) | 87.42 | (81.14, 94.20) | 10.09% |
| (ng · h/mL) | | | |
| Cmax | 64.16 | (49.82, 82.62) | 34.17% |
| (ng/mL) | | | |

AUC(0-inf) = area under the concentration-time curve from 0 to infinite time
AUC(0-tlast) = area under the concentration-time curve from 0 to the time of the last quantifiable concentration
Cmax = maximum observed concentration
^{a}Ratio between adjusted geometric means (fed/fasted)

Conclusion:
Compared to the fasted condition, a standard high-fat meal decreased the absorption rate of grapiprant tablets as indicated by a 36% decrease in Cmax and an approximately 3-hour delay in Tmax. The systemic exposure (as measured by AUC) was not affected by food.
A 375-mg dose of grapiprant tablets was well-tolerated under both fasted and fed conditions.

### Example 7. A Phase 1b/2 Study of Grapiprant, an EP4 Inhibitor, and Pembrolizumab, a PD-1 Checkpoint Inhibitor, in Patients with Advanced or Metastatic Post-PD-1/L1 Non-Small Cell Lung Cancer (NSCLC) adenocarcinoma

Overall Design: This study is a multi-center, open-label, single-arm, Phase1b/2 study to evaluate the safety and efficacy of grapiprant in combination with pembrolizumab in adult patients diagnosed with NSCLC who have been previously treated for a minimum of 12 weeks with any PD-1 or PD-L1 checkpoint inhibitor. Participant enrollment and continuous safety assessment will be dictated by an mTPI model. Decisions for dose escalation and de-escalation will be made by a safety review board (SRB) comprised of enrolling study investigators and the Sponsor. The starting grapiprant dose will be 300 mg twice a day (BID) unless lowered at the study initiation by the SRB. Dose escalation and confirmation will end after 14 participants have been treated at any of the selected doses found to be acceptable. Following the continuous safety assessment phase, additional participants up to a total trial size of 25 will be enrolled to assess efficacy. Participants, including those who achieve a complete response (CR), may receive treatment with grapiprant and pembrolizumab for up to 2 years or until they experience disease progression with clinical deterioration, unacceptable toxicity, or consent withdrawal, followed by 30- and 90-Day End of Treatment Follow-up visits after their last day of study treatment.
Participants will be treated with grapiprant and pembrolizumab on Cycle 1 Day 1.
PK samples will be taken as indicated on the Schedule of Events (SoE).
Scans for tumor assessment will be assessed for all participants every 8 weeks (+/−7 days) from treatment initiation for the first 3 cycles, and then every 12 weeks (+/−7 days) thereafter, and at the discretion of the investigator.
Participants will be instructed to maintain a normal diet during the Combination Treatment and will be encouraged to take grapiprant with food regularly as food is known to decrease common mild GI AEs in drugs of a similar class (COX-2 inhibitors). Morning food intake will be recorded in the medication administration diary on days when post-dose PK samples are drawn.
Mandatory tumor biopsies will be collected in a subset of up to 10 evaluable participants deemed safe for repeated biopsies before Cycle 1 Day 1 and between the end of Cycle 1 and end of Cycle 3, ideally from the same tumor. A third tumor biopsy will be collected in any participant in the biopsy subgroup who has a partial response on tumor assessment, within a month of RECIST v1.1 response documentation, if safe to access, and discussed with the Sponsor.

Main Inclusion Criteria:
1. Male and female adult patients (≥18 years of age on day of signing informed consent) with a histologically confirmed non-small cell lung cancer (NSCLC) adenocarcinoma.
2. Advanced (stage IIIb) disease that is not amenable to curative intent treatment with concurrent chemoradiation and metastatic (stage IV) patients. There is no limit to the number of prior treatment regimens.
3. Patients must have progressed clinically and/or radiographically per RECIST v1.1 after receiving a PD-1 or PD-L1 antagonist for a minimum of 12 weeks. Note: Immunotherapy may have been given with or without chemotherapy and may have been used in any line, however no more than one prior regimen of immunotherapy is allowed.
4. Have measurable disease per RECIST v1.1 as assessed by the local site investigator/radiology. Lesions situated in a previously irradiated area are considered measurable if progression has been demonstrated in such lesions.
5. For biopsy subgroup (10 participants), disease that can be safely accessed via bronchoscopic, thoracoscopic or percutaneous biopsy for multiple core biopsies (minimum of 3 passes per biopsy) and participant is willing to provide tissue from newly obtain biopsies on study.
6. Have an Eastern Cooperative Oncology Group (ECOG) performance status of 0 to 1.
7. Have adequate organ function as defined in Table A below.
8. Willing to use contraception for women who are not postmenopausal and all men.
9. Be willing and able to provide written informed consent for the trial.

TABLE A
Adequate Organ Function Laboratory Values

| System | Laboratory Value |
|---|---|
| Hematological | |
| ANC | ≥1500/μL |
| Platelets | ≥75,000/μL |
| Hemoglobin | ≥9.0 g/dL or ≥5.6 mmol/L¹ |
| Renal | |
| Creatinine OR | ≤1.5 × ULN OR |
| Measured or calculated² creatinine | ≥40 mL/min for participant with creatinine |
| clearance in mL/min | levels |
| (GFR can also be used in place of | >1.5 × institutional ULN |
| creatinine or CrCl) | |
| Hepatic | |
| Total bilirubin | ≤1.5 × ULN OR direct bilirubin ≤ULN for |
| | participants with total bilirubin levels >1.5 × |
| | ULN |
| AST (SGOT) and ALT (SGPT) | ≤2.5 × ULN (≤5 × ULN for participants with |
| | liver metastases) |
| Coagulation | |
| INR OR PT | ≤1.5 × ULN unless participant is receiving |
| aPTT | anticoagulant therapy as long as PT or aPTT |
| | is within therapeutic range of intended use of |
| | anticoagulants |

ALT (SGPT) = alanine aminotransferase (serum glutamic pyruvic transaminase); ANC = absolute neutrophil count; aPTT = activated partial thromboplastin time; AST (SGOT) = aspartate aminotransferase (serum glutamic oxaloacetic transaminase); CrCl = creatinine clearance; GFR = glomerular filtration rate; INR = international normalized ratio; PT = prothrombin time; ULN = upper limit of normal.
¹Criteria must be met without erythropoietin dependency and without packed red blood cell (pRBC) transfusion within last 2 weeks.
²Creatinine clearance in ml/min should be estimated by Cockcroft-Gault formula.
Note:
This table includes eligibility-defining laboratory value requirements for treatment; laboratory value requirements should be adapted according to local regulations and guidelines for the administration of specific chemotherapies.

Main Exclusion Criteria:
1. Current use of NSAIDs (eg, ibuprophen, naproxen), COX-2 inhibitors (eg, celecoxib) within 3 days before treatment initiation or at any time during the study unless used for management of A_{E} or otherwise authorized by the medical director. Aspirin products should be limited to prophylactic cardiovascular doses unless discussed with the Sponsor.
2. Any patient with a known epidermal growth factor receptor (EGFR), anaplastic lymphoma kinase (ALK), or ROS gene alteration.
3. Any patient with a known BRAF gene mutation.
4. Any patient without a history of smoking (≤100 cigarettes lifetime) should be discussed with the Sponsor before enrolling.
5. History of severe hypersensitivity reactions to a PD-1/L1 antibody.
6. Has received prior systemic anti-cancer therapy including investigational agents within 4 weeks prior to treatment. Note: Participants must have recovered from all AEs due to previous therapies to ≤Grade 1 or baseline. Participants with ≤Grade 2 neuropathy may be eligible after discussion with the Sponsor.
7. Has received prior radiotherapy within 2 weeks of start of study treatment. Participants must have recovered from all radiation-related toxicities, not require corticosteroids, and not have had radiation pneumonitis. A 1-week washout is permitted for palliative radiation (≤2 weeks of radiotherapy) to non-central nervous system (CNS) disease.
   Note: No other concurrent antineoplastic treatment is permitted on study except for allowed local radiation of lesions for palliation only (to be considered non-target lesions after treatment)
   Note: If participant received surgery, they must have recovered fully from the toxicity and/or complications from the intervention prior to starting study treatment.
8. Has received a live vaccine within 30 days prior to the first dose of study treatment.
9. Participants taking strong CYP3A4 or P-glycoprotein inhibitors or inducers are excluded from the study unless they can be transferred to other medications within ≥5 half-lives prior to dosing.
10. Is currently participating in or has participated in a study of an investigational agent or has used an investigational device within 4 weeks prior to the first dose of study treatment. Note: Participants who have entered the follow-up phase of an investigational study may participate as long as it has been 4 weeks after the last dose of the previous investigational agent.
11. Has a diagnosis of immunodeficiency or is receiving chronic systemic steroid therapy (in dosing exceeding 10 mg daily of prednisone equivalent) or any other form of immunosuppressive therapy within 7 days prior the first dose of study treatment.
12. Has a known additional potentially life-threatening malignancy that is progressing or has required active treatment within the past 3 years. Note: Participants with basal cell carcinoma of the skin, squamous cell carcinoma of the skin, or carcinoma in situ (eg, breast carcinoma, cervical cancer in situ) that have undergone potentially curative therapy are not excluded.
13. Has known active CNS metastases and/or carcinomatous meningitis (clinically stable and/or previously treated inactive CNS metastases allowed).
14. Has an active autoimmune disease that has required systemic treatment in past 2 years (ie, with use of disease modifying agents, corticosteroids or immunosuppressive drugs). Replacement therapy (eg, thyroxine, insulin, or physiologic corticosteroid replacement therapy for adrenal or pituitary insufficiency) is not considered a form of systemic treatment and is allowed. Autoimmune diseases include but are not limited to inflammatory bowel disease (IBD) such as Crohn's disease and ulcerative colitis.
15. Has a history of (non-infectious) pneumonitis that required steroids or has current pneumonitis.
16. Has an active infection requiring systemic therapy.
17. Recent (within the last 12 months) or current GI ulcer or colitis or non-immune colitis.
18. Has a known history of human immunodeficiency virus (HIV) infection.
19. Has a known history of Hepatitis B or known active Hepatitis C virus infection.
20. Clinically significant (ie, active) cardiovascular disease: cerebral vascular accident/stroke (<6 months prior to enrollment), myocardial infarction (<6 months prior to enrollment), unstable angina, congestive heart failure (≥New York Heart Association Classification Class II), or uncontrolled cardiac arrhythmia.
21. Has a history or current evidence of any condition, therapy, or laboratory abnormality that might confound the results of the study, interfere with the participant's participation for the full duration of the study, or is not in the best interest of the participant to participate, in the opinion of the treating investigator.
22. Has known psychiatric or substance abuse disorders that would interfere with cooperating with the requirements of the study.
23. A woman of childbearing potential (WOCBP) who has a positive pregnancy test prior to treatment.
24. Is breastfeeding or expecting to conceive or father children within the projected duration of the study.

Number of Participants:
Approximately 30 patients from approximately 3 to 6 study centers in the United States will be screened to enroll 25 participants in this study. Enrollment is defined as the time of initiation of the first dose of study treatment.
Participants who are withdrawn from treatment during the first cycle (ie, dose-limiting toxicity [DLT] period) for reasons other than AEs will be replaced

Intervention Groups and Duration:

A cycle of treatment will be defined as every 3 weeks (Q3W).
Participants will receive the combination of grapiprant and pembrolizumab beginning on Cycle 1 Day 1.
The dose of grapiprant will be 300 mg administered orally BID (daily dose taken at 8-to 12-hour intervals, preferably with food).
The pembrolizumab dose will be 200 mg IV Q3W.
Dose and schedule adjustments, corticosteroid administration, and monitoring plan are described in the protocol.
Participants with a DLT within the first cycle will have their dose of both grapiprant and pembrolizumab held until amelioration of their toxicities and be reduced from their existing dose of grapiprant by either 50 mg BID or 100 mg BID unless discussed with the Sponsor.
Participants who experience a first intolerable treatment-emergent adverse event (TEAE) after the first cycle will have their dose of grapiprant and pembrolizumab held until amelioration of their toxicities and be reduced from their existing grapiprant dose by 50 mg BID increments. Switching grapiprant administration to a 2 week on/1 week off schedule is also to be considered by the investigator depending on the nature of the TEAE.
Any participant who requires a decrease in the grapiprant dose below 150 mg BID will have grapiprant treatment discontinued, but may continue to receive pembrolizumab if clinical benefit has been demonstrated.

Participants with Grade 2 or greater dyspepsia for 5 or more days may, at the judgment of the investigator, institute ranitidine at 75 mg orally BID, to be taken 2 hours after the dose of grapiprant, until symptoms abate.

Additional dose adjustment and monitoring plan is described in the protocol.

Participants, including those who achieve a CR, may receive grapiprant and pembrolizumab until they experience disease progression with clinical deterioration, unacceptable toxicity, or consent withdrawal, followed by 30- and 90-Day End of Treatment Follow-up visits after their last day of study treatment.

The duration of the study for each participant will include a Screening period for inclusion in the study of up to 28 days, courses of Combination Treatment cycles repeated every 21 days for a maximum of 35 cycles (up to 2 years), and End of Treatment Follow-up visits at 30 and 90 days following the last day of study treatment administration. The End of Treatment 90-Day Follow-up visit will be considered as the End of Study visit.

Dose de-escalation for all participants will take place any time safety rules indicate (eg, if 4 participants or more participants out of the first 6 participants experiences a DLT). Participants already enrolled and receiving drug without severe AEs may be permitted to receive additional doses at the original dose level after discussion with the Sponsor.

The expected enrollment period is 15 months. The study cut-off date is defined as the date when all the participants have either completed 16 weeks of treatment (ie, until the second tumor assessment) or discontinued the study treatment. The participants who continue to receive the study treatment after the study cut-off date will be followed and appropriate statistical analysis (listings or updated tables for safety, drug exposure and activity) will be performed when all the participants have discontinued the study treatment.

Statistical Considerations:

Determination of the sample size: The combination side-effect profile is expected to be similar to pembrolizumab alone.

The recommend sample size for the mTPI design is n=k*(d+1) (Ji and Wang, J Clin Oncol. 2013; 31(14):1785-91). If 8 subjects are dosed per dose level (k=8) and there are 2 doses tested (d=2), then it is anticipated that 24 subjects will be required. If 1 dose is tested, then it is anticipated that n=16 subjects will be needed. Dose escalation and confirmation will end after 14 participants have been treated at any of the selected doses found to be acceptable. Following the continuous safety assessment phase, additional subjects up to a total trial size of 25 will be assessed to establish an estimate of efficacy. There is no formal hypothesis testing or adjustment for multiplicity.

General statistical approach: Descriptive analysis of safety parameters will be performed on the whole treated population, defined as all participants exposed to at least one dose of grapiprant. Type, frequency, seriousness and relatedness of TEAEs will be analyzed according to Medical Dictionary for Regulatory Activities (MedDRA). Laboratory abnormalities will be analyzed according to National Cancer Institute Common Terminology Criteria for Adverse Events (NCI-CTCAE v5.0).

Pharmacokinetic Analyses: PK parameters of grapiprant will be summarized using descriptive statistics by dose level and time since last dose. The plasma PK of grapiprant will be described for the Cmax and AUC PK parameters. Any additional PK analyses will be described in the statistical analysis plan (SAP).

Population PK and Exposure-Response Analyses: Data from this study will be included with data collected from previous studies in a population PK analysis. The influence of covariates (eg, body weight, age, sex, race, and concomitant medications) on PK parameters will be investigated, if necessary and appropriate.

Additional exploratory PK and/or exposure-response modeling may be applied to the data, as appropriate.

Results of PK and/or any population PK or exposure-response analyses may be reported outside the clinical study report.

Efficacy analysis: Anti-tumor efficacy data will be descriptively presented on the evaluable response population including participants who had a disease assessment at screening and at a minimum one other time point during the study treatment.

The following estimate and confidence intervals (CIs) are meant to provide an overview of the precision of the ORR estimate under several scenarios.

If 1/25 subjects respond, the mean (95% CI) is 0.04 (0.0020, 0.1761).

If 2/25 subjects respond, the mean (95% CI) is 0.08 (0.0144, 0.2310).

If 3/25 subjects respond, the mean (95% CI) is 0.12 (0.0335, 0.2817).

If 4/25 subjects respond, the mean (95% CI) is 0.16 (0.0566, 0.3296).

If 5/25 subjects respond, the mean (95% CI) is 0.20 (0.0823, 0.3754).

If 8/25 subjects respond, the mean (95% CI) is 0.32 (0.1703, 0.5036)

### Example 8. A Phase 1b Study of Grapiprant, an EP4 Inhibitor, and Pembrolizumab, a PD-1 Checkpoint Inhibitor in Patients with Advanced or Progressive Microsatellite Stable (MSS) Colorectal Cancer (CRC)

Overall Design: The study is a multi-center, open-label, single-arm, Phase lb, safety, and efficacy study of grapiprant in combination with pembrolizumab in adult patients with advanced or progressive MSS CRC. This is the first study combining grapiprant with a PD-1 antibody (pembrolizumab), therefore, participant enrollment and continuous safety assessment will be dictated by an mTPI model. The Combination Treatment period will consist of 35 cycles (up to 2 years). The study also includes a one-week Single Agent Run-in period for the purpose of assessing pharmacodynamics of grapiprant as a single agent, as well as in combination with pembrolizumab in the following Combination Treatment period. Participants enrolled into Cohort 1 will be treated with grapiprant during the Single Agent Run-in period and all participants enrolled into Cohort 1 and Cohort 2 will receive treatment with grapiprant and pembrolizumab during the Combination Treatment period. Approximately 30 patients are planned to be screened for this study to allow up to 15 participants for enrollment into Cohort 1 and up to 10 participants for enrollment into Cohort 2. Cohort 1 will enroll participants prior to enrollment of participants into Cohort 2. Following the continuous safety assessment phase, enrollment of additional participants, up to a total trial size of 25 participants, will be assessed to establish an estimate of efficacy.

Single Agent Run-in Period: Cohort 1
Participants will be treated for 1 week with the pharmacologically active dose of grapiprant as a single agent. A starting dose of Grapiprant 300 mg will be administered orally twice a day (BID).
Participants will be instructed to maintain a normal diet during the Single Agent Runin and will be encouraged to take grapiprant with food regularly as food is known to decrease common mild GI AEs in drugs of a similar class (COX-2 inhibitors).
A mandatory pre-treatment tumor biopsy will be collected for participants who are deemed safe for repeated biopsies in Cohort 1 before the first dose of grapiprant on Day 1 and a mandatory post-treatment tumor biopsy will be obtained between Day 5 of the Single Agent Run-in period and pre-dose of pembrolizumab on Cycle 1 Day 1 of the Combination Treatment period, ideally from the same tumor.
PK samples will be taken as indicated on the Schedule of Events (SoE).

Combination Treatment Period: Cohorts 1 and 2
All participants in Cohorts 1 and 2 will be treated with a starting dose of grapiprant 300 mg administered orally BID unless a dose de-escalation occurs and a fixed dose of pembrolizumab administered 200 mg IV every 3 weeks (Q3W) beginning on Cycle 1 Day 1.
PK samples will be taken as indicated on the SoE.
For participants deemed safe for repeated biopsies in Cohort 2, a mandatory pretreatment tumor biopsy will be collected during screening prior to receiving the first dose of either agent on Cycle 1 Day 1 and a mandatory second tumor biopsy will be collected between the end of Cycle 1 and the end of Cycle 3, ideally from the same tumor. A third tumor biopsy will be collected for any participant who has a partial response (PR) on tumor assessment, within a month of Response Evaluation Criteria in Solid Tumors version 1.1 (RECIST v1.1) response documentation, unless a biopsy has already been obtained within a month of the response or otherwise discussed with the medical director.
Scans for tumor assessment will be assessed for all participants (Cohorts 1 and 2) every 8 weeks (+/−7 days) from treatment initiation for the first 3 cycles, and then every 12 weeks (+/−7 days) thereafter, and at the discretion of the investigator.
Participants will be instructed to maintain a normal diet during the Combination Treatment and will be encouraged to take grapiprant with food regularly as food is known to decrease common mild GI AEs in drugs of a similar class (COX-2 inhibitors). Morning food intake will be recorded in the medication administration diary on days when post-dose PK samples are drawn.

Main Inclusion Criteria:

1. Male and female adult patients (≥18 years of age on day of signing informed consent) with a histologically confirmed advanced, metastatic, or progressive CRC that is MSS. Microsatellite stability is based on prior polymerase chain reaction (PCR), Next-Gen sequencing, or immunohistochemistry results per institutional standards.

2. Patient has received at least two prior lines of therapy for advanced or metastatic CRC, at least one of which included fluorouracil. Adjuvant therapy will be counted as a line of therapy only if progression occurs within 6 months of its completion. There is no limit to the number of prior treatment regimens.

3. Have measurable disease per RECIST v1.1 as assessed by the local site investigator/radiology. Lesions situated in a previously irradiated area are considered measurable if progression has been demonstrated in such lesions.

4. Accessible tumor that can be safely accessed for multiple core biopsies and patient is willing to provide tissue from newly obtain biopsies before and during treatment.

5. Have an Eastern Cooperative Oncology Group (ECOG) performance status of 0 to 1.

6. Have adequate organ function as defined in Table A above.

7. Be able to swallow and absorb oral tablets

8. Willing to use contraception for women who are not postmenopausal and all men.

9. Be willing and able to provide written informed consent for the trial.

Main Exclusion Criteria:

1. Has received prior therapy with an anti-PD-1, anti-PD-L1, or anti-PD-L2 agent or with an agent directed to another stimulatory or co-inhibitory T-cell receptor (eg, CTLA-4, OX 40, CD137).

2. Current use of NSAIDs (eg, ibuprophen, naproxen), COX-2 inhibitors (eg, celecoxib) within 3 days before treatment initiation or at any time during the study unless used for management of A_{E} or otherwise authorized by the Sponsor. Aspirin products should be limited to prophylactic cardiovascular doses unless discussed with the Sponsor.

3. History of severe hypersensitivity reactions to chimeric or humanized antibodies.

4. Has received prior systemic anti-cancer therapy including investigational agents within 4 weeks prior to treatment, or 5 half-lives, whichever is shorter. Participants must have recovered from all AEs due to previous therapies to ≤Grade 1 or baseline. Participants with ≤Grade 2 neuropathy may be eligible after discussion with the Sponsor. If participant received major surgery, they must have fully recovered from the toxicity and/or complications from the intervention prior to starting study treatment.

5. Has received prior radiotherapy within 2 weeks of start of study treatment. Participants must have recovered from all radiation-related toxicities, not require corticosteroids, and not have had radiation pneumonitis. A 1-week washout is permitted for palliative radiation (≤2 weeks of radiotherapy) to non-central nervous system (CNS) disease. No other concurrent antineoplastic treatment is permitted on study except for allowed local radiation of lesions for palliation only (to be considered non-target lesions after treatment).

6. Has received a live vaccine within 30 days prior to the first dose of study drug.

7. Participants taking strong CYP3A4 or P-glycoprotein inhibitors or inducers are excluded from the study unless they can be transferred to other medications within ≥5 half-lives prior to dosing.

8. Is currently participating in or has participated in a study of an investigational agent or has used an investigational device within 4 weeks prior to the first dose of study treatment. Participants who have entered the follow-up phase of an investigational study may participate as long as it has been 4 weeks after the last dose of the previous investigational agent.

9. Has a diagnosis of immunodeficiency or is receiving chronic systemic steroid therapy (in dosing exceeding 10 mg daily of prednisone equivalent) or any other form of immunosuppressive therapy within 7 days prior the first dose of study drug.

10. Has a known additional potentially life-threatening malignancy that is progressing or has required active treatment within the past 3 years. Participants with basal cell carcinoma of the skin, squamous cell carcinoma of the skin, or carcinoma in situ (eg, breast carcinoma, cervical cancer in situ) that have undergone potentially curative therapy are not excluded.

11. Has known active CNS metastases and/or carcinomatous meningitis. Participants with previously treated brain metastases may participate provided they are radiologically stable, ie, without evidence of progression for at least 4 weeks by repeat imaging (note that the repeat imaging should be performed during study screening), and/or clinically stable and without requirement of steroid treatment for at least 14 days prior to first dose of study treatment.

12. Has an active autoimmune disease that has required systemic treatment in past 2 years (ie, with use of disease modifying agents, corticosteroids or immunosuppressive drugs). Replacement therapy (eg, thyroxine, insulin, or physiologic corticosteroid replacement therapy for adrenal or pituitary insufficiency) is not considered a form of systemic treatment and is allowed. Autoimmune diseases include but are not limited to inflammatory bowel disease (IBD) such as Crohn's disease and ulcerative colitis.

13. Has a history of (non-infectious) pneumonitis that required steroids or has current pneumonitis.

14. Has an active infection requiring systemic therapy.

15. Recent (within the last 12 months) or current GI ulcer or non-immune colitis.

16. Has a known history of human immunodeficiency virus (HIV) infection.

17. Has a known history of Hepatitis B or known active Hepatitis C virus infection.

18. Clinically significant (ie, active) cardiovascular disease: cerebral vascular accident/stroke (<6 months prior to enrollment), myocardial infarction (<6 months prior to enrollment), unstable angina, congestive heart failure (≥New York Heart Association Classification Class II), or uncontrolled cardiac arrhythmia.

19. Has a history or current evidence of any condition, therapy, or laboratory abnormality that might confound the results of the study, interfere with the participant's participation for the full duration of the study, or is not in the best interest of the participant to participate, in the opinion of the treating investigator.

20. Has known psychiatric or substance abuse disorders that would interfere with cooperating with the requirements of the study.

21. A woman of childbearing potential (WOCBP) who has a positive pregnancy test prior to treatment.

22. Is breastfeeding or expecting to conceive or father children within the projected duration of the study.

Number of Participants: Approximately 30 patients from approximately 3 to 5 study centers in the United States will be screened to enroll 25 participants (15 participants in Cohort 1 and 10 participants in Cohort 2) in this study. Enrollment is defined as the time of initiation of the first dose of study drug. Participants who are withdrawn from treatment during the Single-Agent Run-in (Cohort 1) or the first cycle of combination (ie, dose-limiting toxicity [DLT] period) for reasons other than AEs will be replaced.

Treatment Groups and Duration:

A cycle of treatment will be defined as Q3W.

The pembrolizumab dose will be 200 mg IV Q3W.
Dose and schedule adjustments, corticosteroid administration, and monitoring plan are described in the protocol.

The dose of grapiprant will be 300 mg administered orally BID (daily dose taken at 8-to 12-hour intervals, preferably with food).
Participants with a DLT within the first cycle will have their dose held until amelioration of their toxicities and be reduced from their existing dose by 50 mg BID or 100 mg BID unless discussed with the Sponsor.
Participants who experience a first intolerable treatment-emergent adverse event (TEAE) after the first cycle will have their dose held until amelioration of their toxicities and be reduced from their existing dose at 50 mg BID increments. Switching grapiprant administration to a 2 week on/1 week off schedule is also to be considered by the investigator depending on the nature of the TEAE.
Any participant who requires a decrease in the grapiprant dose below 150 mg BID will have grapiprant treatment discontinued, but may continue to receive pembrolizumab if clinical benefit has been demonstrated.

Participants with Grade 2 or greater dyspepsia for 5 or more days may, at the judgment of the investigator, institute ranitidine at 75 mg orally BID, to be taken 2 hours after the dose of grapiprant, until abdominal discomfort abates.

Additional dose adjustment and monitoring plan is described in the protocol.

Participants, including those who achieve a complete response (CR), may receive treatment with grapiprant and pembrolizumab for up to 2 years or until they experience disease progression, unacceptable toxicity, or consent withdrawal, followed by 30- and 90-Day End of Treatment Follow-up visits after their last day of study drug.

The duration of the study for each participant will include a screening period for inclusion in the study of up to 28 days, a 7-day Single Agent Run-in (for Cohort 1 only), courses of Combination Treatment cycles repeated every 21 days, and End of Treatment Follow-up visits at 30 and 90 days following the last study drug administration for all participants. Participants may continue to receive the study drugs for a maximum of 35 cycles (up to 2 years).

Dose de-escalation for all participants will take place any time safety rules indicate (eg, if 3 or more participants out of the first 5 participants experiences a DLT). Participants already enrolled and receiving drug without severe AEs may be permitted to receive additional doses at the original dose level after discussion with the Sponsor.

The expected enrollment period is 10 months. The study cut-off date is defined as the date when all the participants have either completed 16 weeks of treatment (ie., until the second tumor assessment) or discontinued the study drug. The participants who continue to receive the study drug after the study cut-off date will be followed and appropriate statistical analysis (listings or updated tables for safety, drug exposure and activity) will be performed when all the participants have discontinued the study drug.

Statistical Considerations:

Determination of the sample size: The combination side-effect profile is expected to be similar to pembrolizumab alone.

The recommend sample size for the mTPI design is n=k*(d+1) (Ji and Wang, J Clin Oncol. 2013; 31(14):1785-91). If 8 subjects are dosed per dose level (k=8) and there are 2 doses tested (d=2), then it is anticipated that 24 subjects will be required. If 1 dose is tested, then it is anticipated that n=16 subjects will be needed. Following the continuous safety assessment phase, additional subjects up to a total trial size of 25 will be assessed to establish an estimate of efficacy. There is no formal hypothesis testing or adjustment for multiplicity.

General statistical approach: Descriptive analysis of safety parameters will be performed on the whole treated population, defined as all participants exposed to at least one dose of grapiprant. Specifically, both study cohorts will be pooled, and by-cohort analyses will not be performed. Type, frequency, seriousness and relatedness of TEAEs will be analyzed according to Medical Dictionary for Regulatory Activities (MedDRA). Laboratory abnormalities will be analyzed according to National Cancer Institute Common Terminology Criteria for Adverse Events (NCI-CTCAE) v5.0.

Pharmacokinetic Analyses: PK parameters of grapiprant will be summarized using descriptive statistics by dose level and time since last dose. The plasma PK of grapiprant will be described for the Cmax and AUC PK parameters. Any additional PK analyses will be described in the statistical analysis plan (SAP).

Population PK and Exposure-Response Analyses: Data from this study will be included with data collected from previous studies in a population PK analysis. The influence of covariates (eg, body weight, age, sex, race, and concomitant medications) on PK parameters will be investigated, if necessary and appropriate.

Additional exploratory PK and/or exposure-response modeling may be applied to the data, as appropriate.

Results of PK and/or any population PK or exposure-response analyses may be reported outside the clinical study report.

Efficacy analysis: Anti-tumor efficacy data will be descriptively presented on the evaluable response population including participants who had a disease assessment at screening and at a minimum one other time point during the study treatment.

An informal interim analysis will be conducted to enable future trial planning at the Sponsor's discretion and data will be examined on a continuous basis to allow for dose finding decisions.

### Example 9. Anti-Tumor Activity of Compound B in the CT-26 Colon Adenocarcinoma Mouse Model

Compound B has the following formula, or a pharmaceutically acceptable salt thereof, and is an EP4 receptor selective antagonist (see, for example, U.S. Pat. No. 7,238,714).

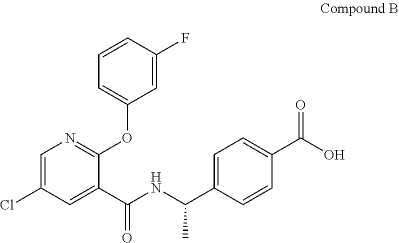 [SMILES: C[C@H](NC(=O)c1cc(Cl)cnc1Oc1cccc(F)c1)c1ccc(C(=O)O)cc1]

The antitumor activity of Compound B as a single agent and combined with a mouse anti-PD-1 antibody was evaluated in the CT-26 mouse colon adenocarcinoma model grown in BALB/c mice. Mice were inoculated subcutaneously in the right flank with 5×10⁵ tumor cells. When tumors reached an average size of 71 mm³ (6 days after tumor cell inoculation) dosing was initiated. The dosing regimens in the 8 separate cohorts comprising 10 mice each are as follows:

| | | | Dose volume | Dosing | |
|---|---|---|---|---|---|
| | | Dose per | per | Schedule/ | Administration |
| Group | Treatment | administration | administration | Days | route |
| 1 | Vehicle(0.5%MC) | — | 10 ul/g | BID × 3+ weeks | p.o. |
| | Rat IgG2(Isotype | 10 mg/kg | 10 ul/g | Day 1, 4, 8, 11, | i.p. |
| | matched) | | | 15 | |
| 2 | Anti-PD1 | 10 mg/kg | 10 ul/g | Day 1, 4, 8, 11, | i.p. |
| | | | | 15 | |
| 3 | Compound B | 15 mg/kg | 10 ul/g | QD × 3+ weeks | p.o. |
| 4 | Compound B | 15 mg/kg | 10 ul/g | BID × 3+ weeks | p.o. |
| 5 | Compound B | 15 mg/kg | 10 ul/g | QD × 3+ weeks | p.o. |
| | Anti-PD1 | 10 mg/kg | 10 ul/g | Day 1, 4, 8, 11, | i.p. |
| | | | | 15 | |
| 6 | Compound B | 15 mg/kg | 10 ul/g | BID × 3+ weeks | p.o. |
| | Anti-PD1 | 10 mg/kg | 10 ul/g | Day 1, 4, 8, 11, | i.p. |
| | | | | 15 | |

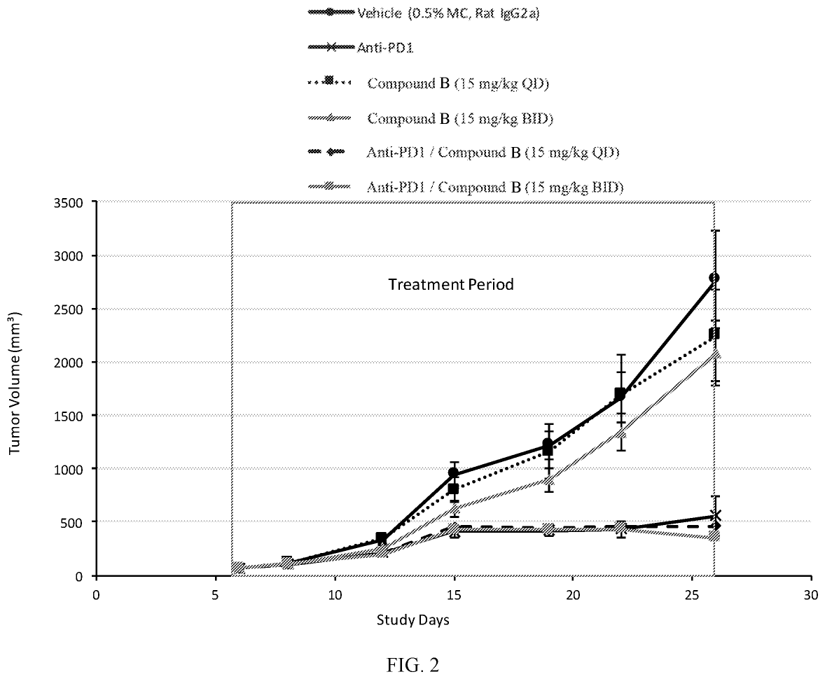

During the period of dosing, the tumor growth kinetics in mice treated with Compound B dosed at 15 mg/kg once daily (QD) and BID were not notably different from the vehicle treated mice (FIG. 2). The tumor growth kinetics in mice treated with Compound B dosed at 15 mg/kg QD and BID combined with anti-PD-1 were also not notably different mice treated with single agent anti-PD-1 during the treatment period. Each dosing regimen was tolerated by the mice as indicated by an average increase in body weight in each cohort during the treatment period and after treatment was discontinued.

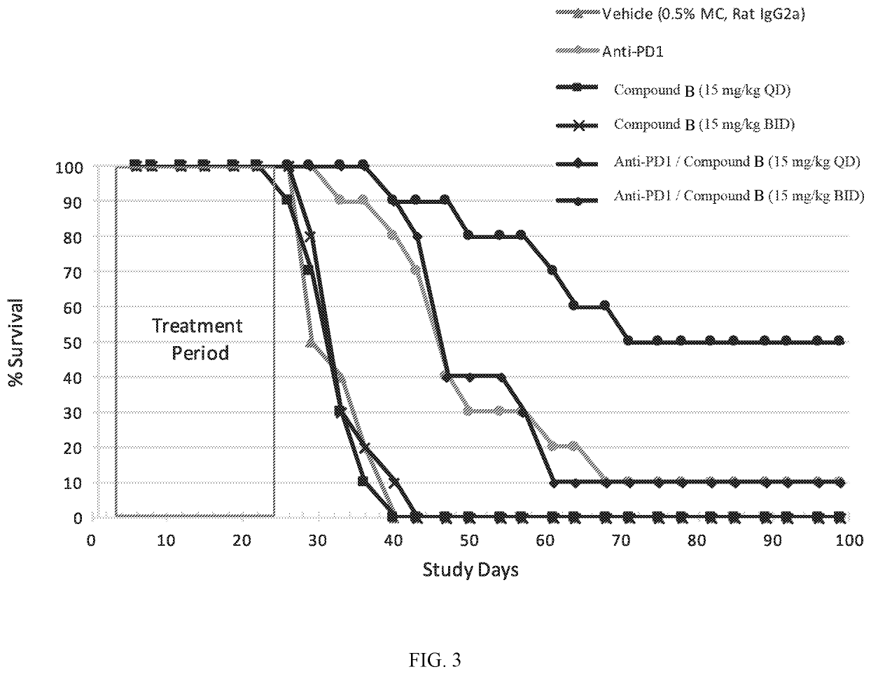

After treatment was discontinued, mice treated with Compound B at 15 mg/kg BID in combination with anti-PD-1 demonstrated decreased growth kinetics relative to anti-PD-1 (FIG. 22) and improved survival (FIG. 3). After continuing to monitor the mice for 99 days after tumor inoculation, 5 out of 10 mice were still alive (4 tumor free) whereas only 1 out of 10 mice treated with anti-PD-1 as a single agent and 1 out of 10 mice in the Compound B was still alive and tumor free. These data suggest that Compound B when combined with anti-PD-1 leads to an improved long term antitumor response.

CT26 tumor cells were inoculated into 6 tumor-naive mice or complete responders of CT26 tumor-bearing mice previously treated with Compound B and anti-PD-1 alone or in combination. The data show that the mice with a complete response decreased the growth of CT26 relative to naive mice suggesting there was a vaccinal effect in the cured mice.

The antitumor activity of Compound B as a single agent and combined with a mouse anti-PD1 antibody was evaluated in the CT-26 mouse colon adenocarcinoma model grown in BALB/c mice in an additional experiment. Mice were inoculated subcutaneously in the right flank with 5×10⁵ tumor cells. When tumors reached an average size of 91 mm³ dosing was initiated. The dosing regimens in the 4 separate cohorts comprising 7 mice each are as follows:

| | | | Dose volume | Dosing | |
|---|---|---|---|---|---|
| | | Dose per | per | Schedule/ | Administration |
| Group | Treatment | administration | administration | Days | route |
| 1 | Vehicle | — | 10 ul/g | BID × 17 days | po |
| | (0.5&MC) | | | | |
| | PBS | — | 10 ul/g | BIW × 4 doses | ip |
| 2 | Compound B | 15 mg/kg | 10 ul/g | BID × 17 days | po |
| | PBS | — | 10 ul/g | BIW × 4 doses | ip |
| 3 | Anti-PD1 | 10 mg/kg | 10 ul/g | BIW × 5 doses | ip |
| | Vehicle | — | 10 ul/g | BID × 16 days | po |
| 4 | Compound B | 15 mg/kg | 10 ul/g | BID × 17 days | po |
| | Anti-PD1 | 10 mg/kg | 10 ul/g | BIW × 5 doses | ip |

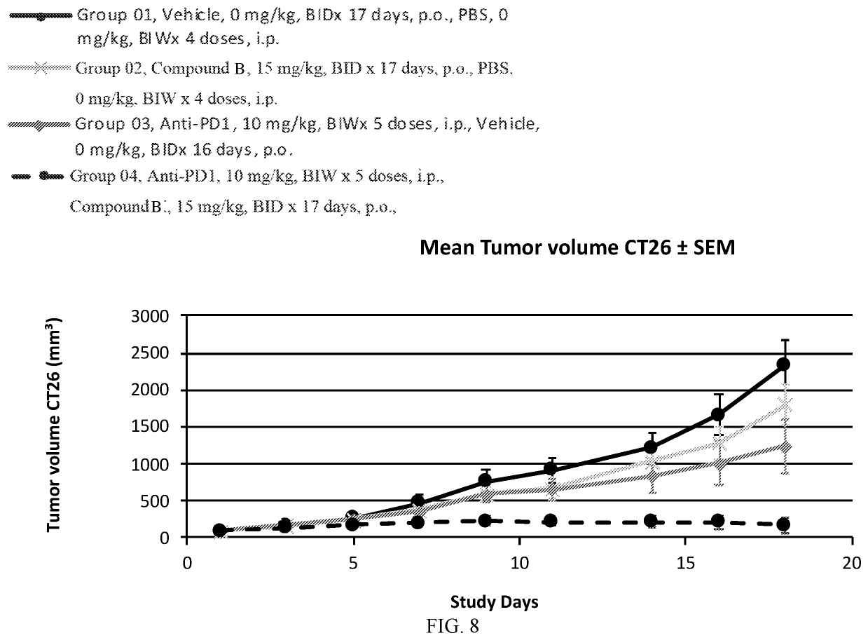

During the period of dosing, the tumor growth kinetics in mice treated with Compound B dosed at 15 mg/kg twice daily (BID) and anti-PD1 were less than that of the vehicle group (FIG. 8). The tumor growth kinetics in mice treated with Compound B dosed at 15 mg/kg BID combined with anti-PD1 were lower than mice treated with either single agent. Each dosing regimen was tolerated by the mice as indicated by an average increase in body weight in each cohort during the treatment period.

### Example 10. Anti-Tumor Activity of Compound B in the 4T1 Breast Cancer Mouse Model

The antitumor activity of Compound B (as described in example 9 above) as a single agent and combined with a mouse anti-CTLA4 antibody was evaluated in the 4T1 mouse breast cancer model grown in BALB/c mice. Mice were inoculated subcutaneously in the right flank with 3×10⁵ tumor cells. When tumors reached an average size of 100 mm³ (7 days after tumor cell inoculation) dosing was initiated.

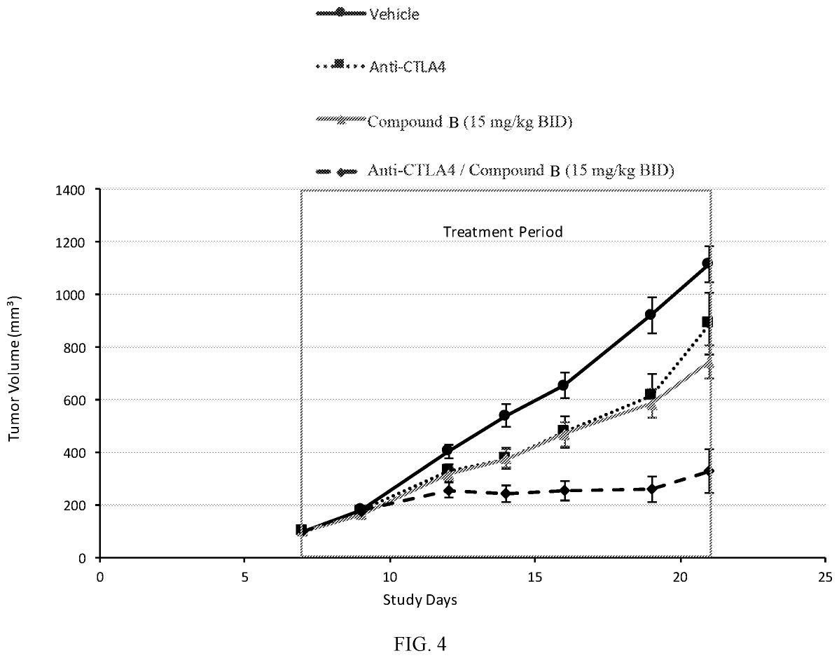

During the period of dosing, the tumor growth kinetics in mice treated with Compound B dosed at 15 mg/kg BID and anti-CTLA4 were decreased relative to the vehicle treated mice (FIG. 4). Moreover, the tumor growth kinetics in mice treated with Compound B and anti-CTLA4 combined was decreased relative to either agent when dosed alone. Each dosing regimen was tolerated by the mice as indicated by an average increase in body weight in each cohort during the treatment period and after treatment was discontinued.

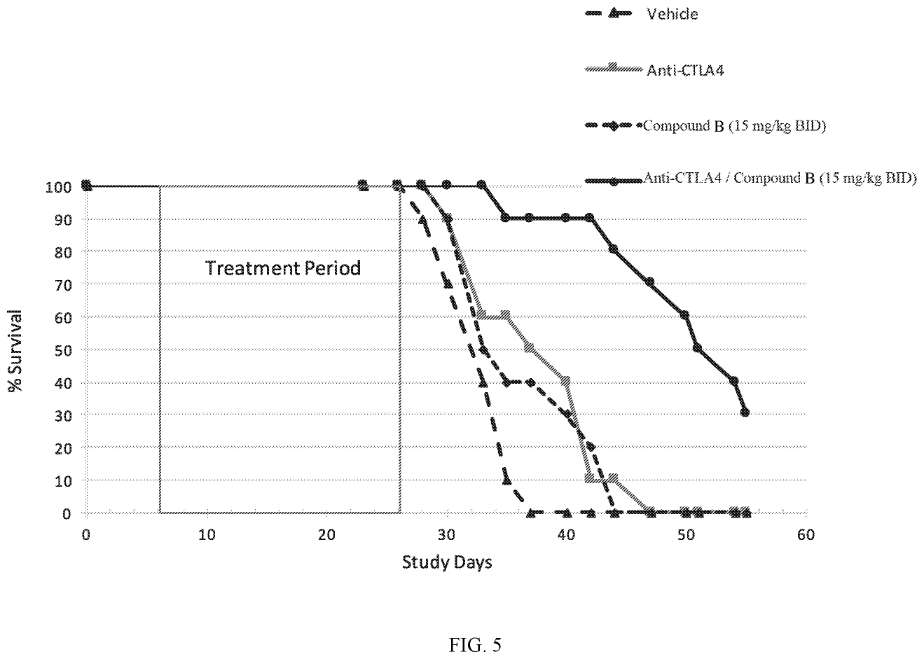

After treatment was discontinued, mice treated with Compound B at 15 mg/kg BID in combination with anti-CTLA4 demonstrated improved survival rate relative to either single agent alone (FIG. 5). For example, after continuing to monitor the mice for 47 days after tumor inoculation, 7 of 10 mice treated with the combination were still alive whereas none of mice treated with either single agent alone was alive 47 days after tumor inoculation. 3 of 10 mice treated with the combination were still alive at the end of the study 55 days after tumor inoculation. These data suggest that the Compound B and anti-CTLA4 combination leads to an improved antitumor response relative to either agent alone.

The antitumor activity of Compound B as a single agent and combined with a mouse anti-PD1 antibody was evaluated in the 4T1 mouse breast cancer model grown in BALB/c mice in an additional experiment. Mice were inoculated subcutaneously in the right flank with 3×10⁵ tumor cells. When tumors reached an average size of 97 mm³ dosing was initiated. The dosing regimens in the 4 separate cohorts comprising 7 mice each are as follows:

| | | | Dose volume | Dosing | | |
|---|---|---|---|---|---|---|
| | | Dose per | per | Schedule/ | | Administration |
| Group | Treatment | administration | administration | Days | | route |
| 1 | Vehicle | — | 10 ul/g | BID × 20 | days | po |
| | (0.5&MC) | | | | | |
| | PBS | — | 10 ul/g | BIW × 5 | doses | ip |
| 2 | Compound B | 15 mg/kg | 10 ul/g | BID × 20 | days | po |
| | PBS | — | 10 ul/g | BIW × 5 | doses | ip |
| 3 | Anti-PD1 | 10 mg/kg | 10 ul/g | BIW × 6 | doses | ip |
| | Vehicle | — | 10 ul/g | BID × 19 | days | po |
| 4 | Compound B | 15 mg/kg | 10 ul/g | BID × 20 | days | po |
| | Anti-PD1 | 10 mg/kg | 10 ul/g | BIW × 6 | doses | ip |

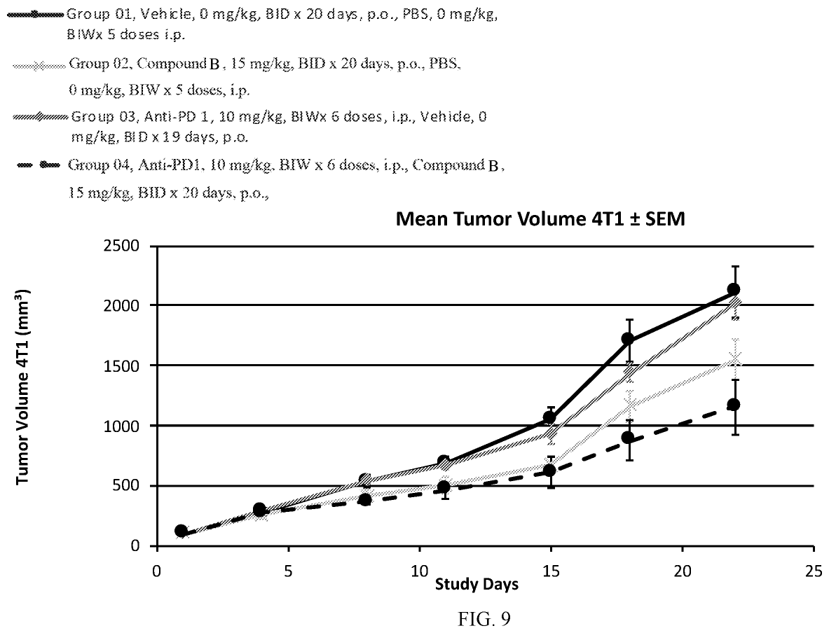

During the period of dosing, the tumor growth kinetics in mice treated with Compound B dosed at 15 mg/kg twice daily (BID) was less than that of the vehicle group and anti-PD1 when dosed alone (FIG. 9). The tumor growth kinetics in mice treated with Compound B dosed at 15 mg/kg BID combined with anti-PD1 were lower than mice treated with either single agent. Each dosing regimen was tolerated by the mice as indicated by an average increase in body weight in each cohort during the treatment period.

### Example 11. Manufacture of Grapiprant Unit Dosage Form OPC and Grapiprant Liquid Unit Dosage Form

### 11.1 Grapiprant Unit Dosage Form OPC

Grapiprant unit dosage form OPC at a strength of 10 mg, 30 mg, 60 mg, 100 mg, 200 mg, 300 mg, 600 mg, 1000 mg, 1500 mg, and 2000 mg are prepared, based on a theoretical drug substance potency of 100.0%. The component for the grapiprant unit dosage form OPC is listed in Table 11-1.

TABLE 11-1
Grapiprant Unit Dosage Form OPC Components.

| | |
|---|---|
| Grapiprant unit dosage | |
| form OPC Strength | Component |

| | | | |
|---|---|---|---|
| 10 | mg | 10 | mg Grapiprant |
| 30 | mg | 30 | mg Grapiprant |
| 60 | mg | 60 | mg Grapiprant |
| 100 | mg | 100 | mg Grapiprant |
| 200 | mg | 200 | mg Grapiprant |
| 300 | mg | 300 | mg Grapiprant |
| 600 | mg | 600 | mg Grapiprant |
| 1000 | mg | 1000 | mg Grapiprant |
| 1500 | mg | 1500 | mg Grapiprant |
| 2000 | mg | 2000 | mg Grapiprant |

Grapiprant unit dosage form OPC are prepared according to the following procedure:
1 The appropriate amount of grapiprant is weighed and transferred into a previously cleaned 4 ounce amber glass bottle.
2 Each of the filled bottles from Step 1 is sealed with a previously cleaned closure and torqued.

### 11.2 Vehicle and Placebo for Grapiprant Unit Dosage Form OPC and Grapiprant Liquid Unit Dosage Form

TABLE 11-2
Vehicle and Placebo Component/Composition

| | Unit Strength |
|---|---|
| | 0 mg |
| Component | (mg/bottle) |
| Hydroxypropyl cellulose | 200.0 |
| Microcrystalline cellulose | 200.0 |
| Microcrystalline cellulose | 100.0 |
| Titanium dioxide | 50.0 |
| Xylitol | 400.0 |
| 30% Simethicone Emulsion | 33.3 |
| Total | 983.3 |

1 The appropriate amounts of xylitol and hydroxypropylcellulose are added to an appropriate size mortar and blended.
2 The appropriate amount of xylitol and the remaining hydroxypropylcellulose are added to mortar from step 1 and blended.
3 The remaining xylitol is added to the mortar from step 2 and blended.
4 The titanium dioxide and the appropriate amount of microcrystalline cellulose are added to an appropriate size mortar and blended.
5 The remaining microcrystalline cellulose is added to the mortar from step 4 and blended.
6 The appropriate amount of microcrystalline cellulose and the 30% simethicone emulsion are added to an appropriate size weighing dish.
7 The appropriate amounts of microcrystalline cellulose and the contents of the weighing dish from step 6 are added to an appropriate size mortar and blended.
8 The remaining microcrystalline cellulose is added to the mortar from step 7 and blended.
9 The blends from step 3, step 5 and from step 8 are added to an appropriate size bottle and blended.
10 The appropriate amount of blend from step 9 is weighed and transferred into a previously cleaned 4-ounce amber glass bottle.
11 Each of the filled bottles from step 10 is sealed with a previously cleaned closure and torqued.

### 11.3 Preparation, Constitution, and Administration Instructions

### 11.3.1 Preparation of 0.3, 1, 3 and 10 mg Grapiprant Liquid Unit Dosage Forms

1 Obtain a 4 oz. amber bottle containing 10 mg grapiprant. This is Bottle S.
2 Tap Bottle S gently in the upright position to dislodge any powder trapped in the neck or cap and loosen the powder from the bottom of the bottle
3 Using a 60 cc plastic syringe, accurately transfer 100 mL (50 mL+50 mL) SWI to Bottle S, containing grapiprant. Recap the bottle.
4 Shake Bottle S vigorously for 4 minutes to dissolve the contents. If the contents of Bottle S are not used within 4 hours, discard the bottle and its contents.
5 If the dose is 10 mg then stop after step 4. If the dose is 0.3, 1 or 3 mg, continue on to step 6.
6 Use Table 11-3 to determine the volumes of the 10 mg Grapiprant Stock Solution in Bottle S and SWI to be used for preparation of the desired doses.
7 Using an appropriately sized syringe (according to Table 11-3), transfer the appropriate volume of the 10 mg Grapiprant Stock Solution in Bottle S to an empty 4 oz amber bottle.
8 Discard the 10 mg Grapiprant Stock Solution from which the aliquot was drawn.
9 Using an appropriately sized syringe (according to Table 11-3), transfer the appropriate volume of SWI to the 4 oz amber bottle containing the grapiprant stock solution from step 7. Cap the bottle tightly. This is Bottle D.
10 Invert Bottle D from step 9 five times and label the bottle appropriately. If the contents of Bottle D are not used within 4 hours, discard the bottle and its contents.

TABLE 11-3
Syringes and Volumes of the 10 mg Grapiprant
Stock Solution and SWI for Making 0.3, 1 and
3 mg Grapiprant Liquid Unit Dosage Forms

| | Volume of 10 mg Grapiprant | | | |
|---|---|---|---|---|
| | Stock Solution | | Volume of SWI | |
| Grapiprant | Syringe Size | Volume of 10 mg | Syringe | Volume of |
| (mg) | (cc) | Grapiprant Stock | Size (cc) | SWI |
| 0.3 | 3 | 3 | 60 | 97 |
| 1 | 10 | 10 | 60 | 990 |
| 3 | 10 | 30 | 60 | 70 |

### 11.3.2 Preparation of 30, 60, 100, 200, 300, 600, 1000, 1500, and 2000 mg Grapiprant Liquid Unit Dosage Forms

1 Obtain a 4 oz. amber bottle containing the vehicle. This is Bottle V.
2 Tap Bottle V gently in the upright position to dislodge any powder trapped in the neck or cap and loosen the powder from the bottom of the bottle.
3 Using a 60 cc plastic syringe, accurately transfer 100 mL (50 mL+50 mL) SWI to Bottle V, containing the vehicle. Recap the bottle.
4 Shake Bottle V for 2 minutes and let sit for 1 minute.
5 Obtain a 4 oz. amber bottle containing 30, 60, 100, 200, 300, 600, 1000, 1500 or 2000 mg of grapiprant. This is Bottle A.
6 Tap Bottle A gently in the upright position to dislodge any powder trapped in the neck or cap and loosen the powder from the bottom of the bottle.
7 Invert Bottle V, containing the vehicle, three (3) times and pour the contents into Bottle A, containing grapiprant
8 Cap Bottle A and shake for 1 minute. If the contents of Bottle A are not used within 4 hours, discard the bottle and its contents.

### 11.3.3 Preparation of Placebos for 0.3, 1, 3, and 10 mg Grapiprant Liquid Unit Dosage Forms

1. Obtain an empty, 4 oz. amber bottle. This is Bottle W.
2. Using a 60 cc plastic syringe, accurately transfer 100 mL SWI to Bottle W.

### 11.3.4 Preparation of Placebos for 30, 60, 100, 200, 300, 600, 1000, 1500, and 2000 mg Grapiprant Liquid Unit Dosage Forms

1 Obtain a 4 oz. amber bottle containing the vehicle. This is Bottle P.
2 Tap Bottle P gently in the upright position to dislodge any powder trapped in the neck or cap and loosen the powder from the bottom of the bottle.
3 Using a 60 cc plastic syringe, accurately transfer 100 mL (50 mL+50 mL) SWI to Bottle P, containing the vehicle. Recap the bottle.
4 Shake Bottle P for 2 minutes and let sit for 1 minute.

### 11.3.5 Administration Instructions

a. Prior to dosing, administer 2 mints to the subject. Have him dissolve the mints in his mouth, without chewing, for at least 5 minutes.
2 Have the subject drink the entire contents of Bottles S, D, A, W or P.
3 Accurately add 70 mL of SWI to the dosing bottle, shake and administer rinse to the subject.
4 Repeat step 3.
5 Administer 2 mints to the subject. Have him dissolve the mints in his mouth, without chewing, for at least 5 minutes.

### Example 12. A Phase 1, Randomized, Placebo-Controlled, Sequential Parallel Group Study to Evaluate the Safety, Tolerability and Pharmacokinetics of After First-Time Administration of Single Ascending Doses to Healthy Adult Subjects

Study Objectives: To evaluate the safety, tolerability, and pharmacokinetics of escalating single oral doses of grapiprant administered as OPC in healthy adult subjects.

Study Design: This study was a randomized, investigator-blind, subject-blind, sponsor-open, placebo-controlled, sequential parallel group, single escalating dose study of grapiprant OPC. Eligible subjects were to be enrolled into 8 sequential cohorts, corresponding to the planned dose levels of 1 mg, 3 mg, 10 mg, 30 mg, 100 mg, 300 mg, 600 mg, and 1000 mg. If adequate safety was demonstrated, additional subjects were to be enrolled into 2 optional sequential cohorts (1500 mg and 2000 mg). After determining a maximum tolerated dose (MTD), or after completing the highest dose cohort if a MTD was not determined, Cohort 11 would be enrolled to increase the number of subjects evaluated at the MTD (or the highest dose).

In each cohort, subjects were randomized to receive a single oral dose of grapiprant or matching placebo.

The actual dose of grapiprant OPC (or matching placebo) administered in each cohort could be lower than, but was not to exceed, the planned dose, and was determined based on evaluation of all available safety and pharmacokinetic data. There was a minimum of 7 days between administration of study drug to each ascending dose level cohort.

In each dosing period, subjects were confined to the Clinical Research Unit (CRU) for approximately 60 hours (from approximately 36 hours predose through 24 hours postdose), and were instructed to return to the study site at 48 hours postdose for collection of blood samples for safety and pharmacokinetic evaluations. Subjects had triplicate ECG measurements made at select time-points on Day 0 (to provide a time-matched baseline) and through 24 hours postdose. Continuous cardiac monitoring by telemetry was performed for 12 hours postdose (or up to 24 hours, if warranted, based on the pharmacokinetic data). Creatine phosphokinase (CPK; total CPK and CPK-MB isoenzyme) and troponin I levels were monitored predose and at 12 and 24 hours postdose. In each dosing period, blood samples were collected predose and at 0.5, 1, 1.5, 2, 2.5, 3, 4, 6, 8, 12, 16, 24, and 48 hours postdose for pharmacokinetic evaluation. Fecal occult blood was monitored predose and through 48 hours postdose. Urine was collected through 24 hours postdose for exploratory biotransformation analyses. Since grapiprant may be a substrate of efflux transporters, genotyping for P-gp, an efflux transporter, was also planned for this study.

Study Population and Criteria for Inclusion: Subjects screened for this study were male and female subjects between the ages of 18 and 55 years, inclusive, who had a body weight of >110 lb and a body mass index between 18 to 30 (kg/m2), inclusive. Enrolled subjects were required to be in good health as determined by a detailed medical history, full physical examination (including blood pressure and pulse rate measurements), 12-lead ECG, and clinical laboratory tests.

Treatments are listed below in Table 12-1.

TABLE 12-1
Study Medications Used.

| Study Drugs | Formulation | Dose Unit ^{a} | |
|---|---|---|---|
| grapiprant | OPC | 10 | mg |
| grapiprant | OPC | 30 | mg |
| grapiprant | OPC | 60 | mg |
| grapiprant | OPC | 100 | mg |
| grapiprant | OPC | 200 | mg |
| grapiprant | OPC | 300 | mg |
| grapiprant | OPC | 600 | mg |
| grapiprant | OPC | 1000 | mg |
| grapiprant | OPC | 2000 | mg |
| grapiprant | OPC | 1500 | mg |
| Empty bottle | N/A | N/A | |
| Vehicle/Placebo | Vehicle/Placebo | 0 | mg |

^{a} 10-mg dose unit was diluted for the 1- and 3-mg doses
OPC = oral powder for constitution;
N/A = not applicable

### Criteria for Evaluation and Methodology:

Safety: All subjects were evaluated for safety. Subjects were queried regarding adverse events (AEs) at 24 hours prior to dosing, 0 hours predose (baseline), and 1, 2, 4, 8, and 12 hours postdose. A limited physical examination, clinical laboratory tests, and fecal occult blood test were performed 24 hours prior to dosing. Vital signs measurements and 12-lead ECGs were performed in triplicate (approximately 2 minutes apart) at specified intervals in the 24 hours before dosing, 0 hours (predose), and at 0.5, 1, 1.5, 2, 3, 4, 8, and 12 hours postdose. Continuous cardiac telemetry monitoring was started at 1 hour postdose and continued for 12 hours postdose Fecal occult blood was monitored for 48 hours postdose.

Pharmacokinetics: The maximum observed serum concentration (Cmax) of grapiprant was estimated directly from raw serum concentration-time data with Tmax defined as the time of the first occurrence of Cmax. The apparent terminal elimination rate constant (kel) was estimated using least-squares regression analysis of the serum concentration-time data obtained during the terminal log-linear phase. The apparent terminal half-life (T½) was calculated as In 2/kel. Areas under the serum concentration-time curve from time 0 to the last time (Tlast) with a quantifiable concentration [AUC(Tlast)] were estimated using linear/log trapezoidal approximation. Areas under the serum concentration-time curve from time Tlast to infinity [AUC(Tlast-inf)] were estimated as Cpest/kel, where Cpest represented the estimated concentration at time Tlast based on the aforementioned regression analysis. Areas under the serum concentration-time curve from time 0 to infinity [AUC(inf)] were estimated as the sum of AUC(Tlast) and AUC(Tlast-inf). The arithmetic means were reported for all pharmacokinetic parameters. Individual serum concentrations of grapiprant were summarized in tabular and graphical form. Concentrations that were determined to be below the lower limit of quantification were assumed to be equal to zero for the pharmacokinetic analyses and mean concentration-time plots.

The 600 mg dose was studied in 2 separate cohorts. The second of these two cohorts was conducted to confirm safety assessments at this dose strength. For the pharmacokinetic analysis, pharmacokinetic parameters of both cohorts were pooled together and analyzed as a single group.

Statistical Methods: Since the primary purposes of this study were to evaluate the safety and tolerability and to explore the pharmacokinetics of the study drug, no specific statistical hypothesis tests were planned. Safety and pharmacokinetic data were summarized through appropriate data tabulations, descriptive statistics, and graphical presentation. Placebo subjects from all cohorts were combined as 1 group for data summary and interpretation.

### Results:

Subject Disposition: Eligible subjects were enrolled into the first 7 planned sequential cohorts (dose levels of 1 mg, 3 mg, 10 mg, 30 mg, 100 mg, 300 mg, and 600 mg). One additional cohort of subjects was dosed with 600 mg in order to confirm the safety assessments. No dose-limiting AEs were observed at the first 7 dose levels; therefore, additional subjects were enrolled to receive 1000 mg, 1500 mg, and 2000 mg.

A total of 78 subjects were assigned to receive treatment: 61 subjects received a single dose of grapiprant at doses ranging from 1 mg to 2000 mg, and 17 subjects received placebo. All treated subjects were analyzed for safety and laboratory results. One subject in the placebo treatment group failed to return to the CRU for the postdose follow-up evaluations (to be performed 7 to 10 days following Day 3) and was therefore considered to have discontinued from the study.

Demographic Characteristics: The majority of the subjects were male (95%) and white (79%). Summary data for the enrolled subjects are displayed in Table 12-2.

TABLE 12-2
Demographic Characteristics of the Study Population

| | CJ-023,423 | | | Placebo | | |
|---|---|---|---|---|---|---|
| | Male | Female | Total | Male | Female | Total |
| Number of | 57 | 4 | 61 | 17 | 0 | 17 |
| Subjects | | | | | | |

Age (years)

| | | | | | | |
|---|---|---|---|---|---|---|
| 18-44 | 51 | 2 | 53 | 15 | 0 | 15 |
| 45-64 | 6 | 2 | 8 | 2 | 0 | 2 |
| | 18-55 | 41-55 | 18-55 | 18-46 | 0 | 18-46 |

Race

| | | | | | | |
|---|---|---|---|---|---|---|
| White | 46 | 4 | 50 | 12 | 0 | 12 |
| Black | 9 | 0 | 9 | 4 | 0 | 4 |
| Hispanic | 2 | 0 | 2 | 0 | 0 | 0 |
| Other | 0 | 0 | 0 | 1 | 0 | 1 |
| Weight (kg) | 58.9- | 58.8- | 58.8- | 61.7- | 0 | 61.7- |
| (Range) | 104.4 | 78.2 | 104.4 | 100.0 | | 100.0 |
| Height (cm) | 162.6- | 152.4- | 152.4- | 167.6- | 0 | 167.6- |
| (Range) | 193.0 | 172.7 | 193.0 | 188.0 | | 188.0 |

Safety: No deaths or discontinuations due to AEs were reported in this study. Three subjects experienced SAEs: heart palpitations in 1 subject treated with 600 mg, considered not related to treatment; and 2 subjects (1 subject each at the 1500 mg and 2000 mg dose levels) had mild, treatment-related elevations in serum creatinine and BUN (COSTART term: acute renal failure). A total of 26 of the 61 subjects treated with grapiprant (43%) experienced AEs. The incidence of AEs was highest at the 2 highest dose levels (4 of 6 subjects in the 1500 mg treatment group and 5 of 6 subjects in the 2000 mg treatment group); in general, AEs were mild in severity.

Single doses of grapiprant ranging from 1 mg to 1000 mg were generally well tolerated; no single adverse event occurred in more than 1 subject in a treatment group (with the exception of mild, unrelated chest pain in 2 subjects in the 600 mg treatment group). The most frequently reported A_{E} was abdominal pain, reported in 4 subjects treated with 2000 mg, 2 subjects treated with 1500 mg, and 1 subject treated with 30 mg grapiprant. Additional AEs reported by more than 1 subject in a treatment group were chest pain (2 subjects at 600 mg), nausea (2 subjects at 2000 mg), and taste perversion (2 subjects at 100 mg).

No clinically important, study drug-related changes in vital signs measurements, cardiac telemetry, or ECG parameters were observed. There were no positive fecal occult blood results before or after study drug treatment.

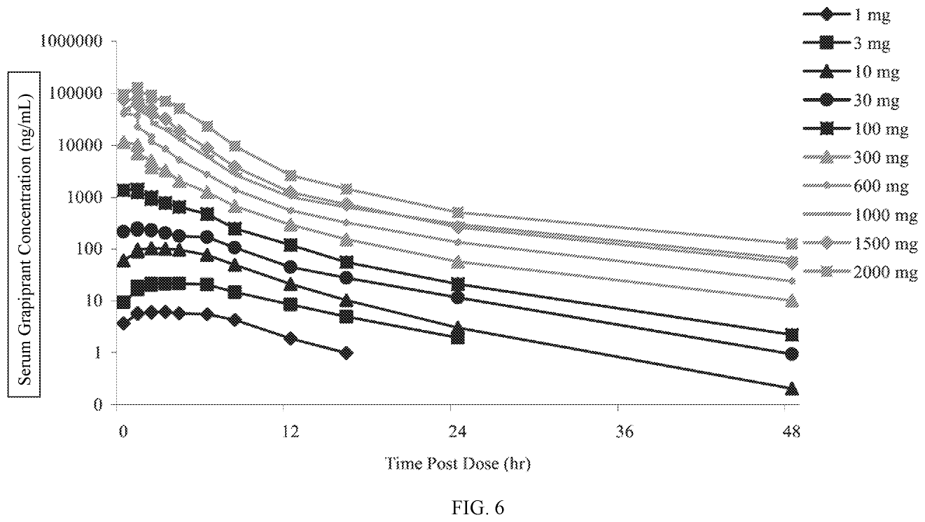

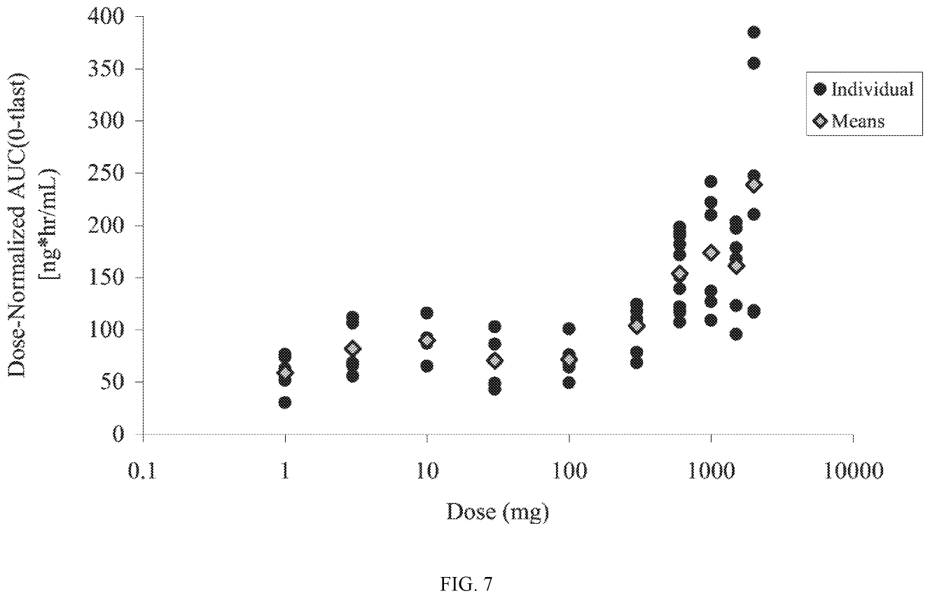

Pharmacokinetics: Following single-dose administration of grapiprant as an OPC to healthy subjects in the fasted state, mean serum exposure parameters increased with dose, with greater than dose-proportional increases observed between the 100 mg and 600 mg dose levels (Table 12-3, FIGS. 6 and 7). Grapiprant exhibited rapid absorption followed by a bi-exponential decline in serum concentration (FIG. 6). Median Tmax estimates ranged between 0.5 and 3 hours across the dose groups, and at doses of 100 mg and above, individual Tmax values ranged from 0.5 hours to 1 hour (Table 12-3). The mean terminal phase T1/2 was comparable over the wide range of doses studied, and ranged from 5.10 to 9.41 hours (Table 12-3). A second phase of the decline in the time-concentration profiles was not evident in the lower dose groups (FIG. 6). In the cohorts with a clear bi-exponential decline in concentration (30 mg and above), the mean terminal phase T1/2 ranged from 6.14 to 9.41 hours.

TABLE 12-3
Mean (SD) Grapiprant Pharmacokinetic Parameters Following Single Doses of Grapiprant
Administered as an OPC in the Fasted State to Healthy Adult Subjects

| | 1 mg | 3 mg | 10 mg | 30 mg* | 100 mg | 300 mg | 600 mg | 1000 mg | 1500 mg | 2000 mg |
|---|---|---|---|---|---|---|---|---|---|---|
| | (n = 5) | (n = 5) | (n = 5) | (n = 5) | (n = 5) | (n = 5) | (n = 5) | (n = 5) | (n = 5) | (n = 5) |
| Cmax | 6.59 | 23.1 | 110 | 262 | 1440 | 12000 | 45000 | 64300 | 83800 | 134000 |
| (ng/mL) | (1.86) | (6.16) | (21.0) | (83.1) | (489) | (3240) | (11500) | (17100) | (21800) | (51600) |
| Tmax | 2.50 | 3.00 | 2.00 | 1.00 | 0.75 | 0.75 | 0.50 | 1.00 | 0.75 | 1.00 |
| (hr) | (1.50- | (2.50- | (1.00- | (1.00- | (0.50- | (0.50- | (0.50- | (0.50- | (0.50- | (0.50- |
| | 8.00) | 6.00) | 2.50) | 2.00) | 1.00) | 1.00) | 1.00) | 1.00) | 1.00) | 1.00) |
| AUC(Tlast) | 59.1 | 246 | 901 | 2120 | 7170 | 31200 | 92200 | 174000 | 242000 | 478000 |
| (ng*hr/mL) | (18.8) | (76.7) | (179) | (750) | (1720) | (7270) | (20700) | (56700) | (64000) | (228000) |
| AUC(inf) | NC | 260 | 917 | 2150 | 7220 | 31300 | 92500 | 175000 | 242000 | 479000 |
| (ng*hr/mL) | (NC) | (82.7) | (178) | (740) | (1720) | (7260) | (20800) | (56700) | (63900) | (229000) |
| T½ | NC | 5.34 | 5.10 | 6.14 | 6.91 | 7.98 | 8.03 | 9.41 | 8.13 | 8.25 |
| (hr) | (NC) | (0.147) | (1.90) | (1.36) | (2.57) | (1.73) | (1.46) | (1.49) | (2.18) | (1.38) |

Median (Range)
NC = not calculated due to insufficient data
*The 12-hr postdose PK sample for a subject in 30 mg cohort was not drawn and was not available for the analyses.

CONCLUSIONS: Subsequent to the administration of a single, oral dose of the OPC formulation, grapiprant exhibited rapid absorption followed by a bi-exponential decline in serum concentration. The exposures and peak serum concentrations of grapiprant increased with dose.

No positive results for fecal occult blood tests, no abnormalities in cardiac telemetry, and no clinically important, treatment-related changes in vital signs measurements or ECG parameters were observed for any treatment groups. For single doses of grapiprant ranging from 1 mg to 1000 mg, adverse events rarely occurred in more than 1 subject in a treatment group. Among the 12 subjects who received a single dose of grapiprant of 1500 or 2000 mg, 6 subjects experienced treatment-related abdominal pain. Two subjects (1 in the 1500 mg treatment group and 1 in the 2000 mg treatment group) experienced mild, asymptomatic, treatment-related elevations in serum creatinine and BUN values (COSTART term: acute renal failure), which returned to within the normal range within 14 days of onset.

### Example 13. Effect of Compound B on Immune Cell Composition in the CT-26 Colon Adenocarcinoma Mouse Model

The immune cell composition of Compound B (as described in example 9 above) as a single agent and combined with a mouse anti-PD1 antibody was evaluated in the CT-26 mouse colon adenocarcinoma model grown in BALB/c mice. Mice were inoculated subcutaneously in the right flank with 5×10⁵ tumor cells. When tumors reached an average size of 85 mm³ dosing was initiated. The dosing regimens in the 4 separate cohorts comprising 10 mice each are listed as follows:

| | | | Dose volume | | Dosing | | |
|---|---|---|---|---|---|---|---|
| | | Dose per | per | | Schedule/ | Administration | Number |
| Group | Treatment | administration | administration | | Days | route | of mice |
| Group 1 | Vehicle | — | 10 | ul/g | BID × 7 | po | 10 |
| | (0.5&MC) | | | | days | | |
| | PBS | — | 5 | ul/g | Q3D × 3 | ip | |
| | | | | | doses | | |
| Group 2 | Compound B | 15 mg/kg | 10 | ul/g | BID × 7 | po | 10 |
| | | | | | days | | |
| | PBS | — | 5 | ul/g | Q3D × 3 | ip | |
| | | | | | doses | | |
| Group 3 | Anti-PD1 | 10 mg/kg | 5 | ul/g | Q3D × 3 | ip | 10 |
| | | | | | doses | | |
| | Vehicle | — | 10 | ul/g | BID × 7 | po | |
| | | | | | days | | |
| Group 4 | Compound B | 15 mg/kg | 10 | ul/g | BID × 7 | po | 10 |
| | | | | | days | | |
| | Anti-PD1 | 10 mg/kg | 5 | ul/g | Q3D × 3 | ip | |
| | | | | | doses | | |

After dosing animals for 7 days, tumors were resected and used to prepare single cell suspensions. Live cells representing tumor and immune cells were stained with cocktails of antibodies targeting multiple immune cell markers (anti-CD45, anti-CD3, anti-CD4, anti-CD8, anti-CD25, anti-FoxP3, anti-PD-1, anti-CD11c) conjugated to different fluorescent tags. The stained cells were fixed in 4% paraformaldehyde and quantified using a multi-color flow cytometer (Fortessa). The data was analyzed with FloJo software.

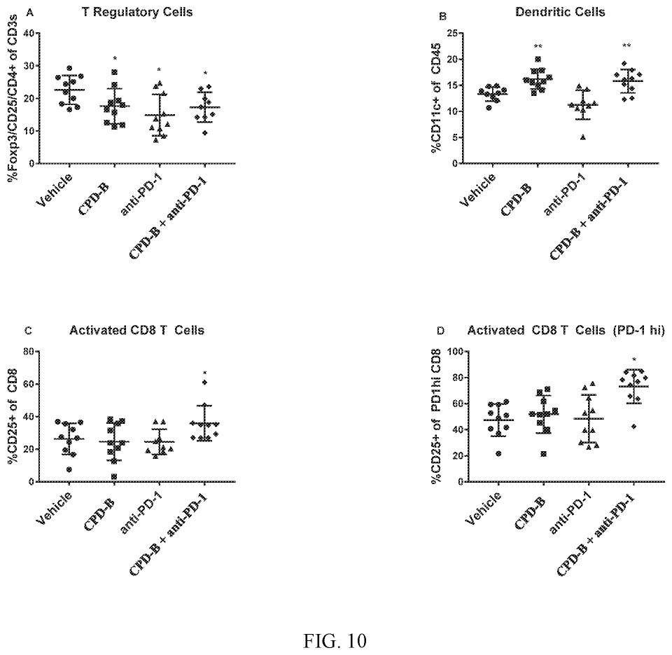

Compound B, anti-PD-1 and the combination of the 2 agents resulted in a significant decrease in regulatory T cells (CD45, CD4, FoxP3, CD25 positive) (FIG. 10a). Compound B dosed alone and with anti-PD-1 led to increased percentage of dendritic cells (CD45, CD11c positive), whereas anti-PD-1 alone did not (FIG. 10b). The combination of Compound B with anti-PD-1 also led to increased percentage of activated T cells (CD45, CD3, CD8) by evaluating the CD25 expression where as either agent dosed alone did not (FIG. 10c). The percentage of CD25 was higher in T cells with increased levels of PD-1 (FIG. 10d). Collectively, these findings demonstrate that Compound B alone and when combined with anti-PD-1 antibodies alters the immune cell composition of CT-26 tumors indicative of an increased proinflammatory phenotype.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A unit dosage form of a pharmaceutical composition comprising about 50 mg to about 375 mg grapiprant, or a pharmaceutically acceptable salt thereof, and one or more pharmaceutically acceptable excipient or carrier.

2. The unit dosage form of claim 1, wherein the pharmaceutical composition comprises about 300 mg of grapiprant, or a pharmaceutically acceptable salt thereof, and one or more pharmaceutically acceptable excipient or carrier.

3. The unit dosage form of claim 2, wherein the unit dosage form, upon a 14-day oral administration in healthy adult subjects as follows:
i) one dose in the morning in a fasting state on day 1;
ii) twice daily (BID, approximately every 12 hours) on days 2 through 13: one dose in a fasting state in the morning, and one dose in the evening on an empty stomach;
iii) one dose in the morning in a fasting state on day 14;
provides one or more pharmacokinetics result on post-dosing day 1 selected from:
a mean serum Cₘₐₓ of about 8240 ng/mL;
a mean serum AUC_{τ} of about 24900 ng*h/mL; and
a mean serum Tₘₐₓ of about 1.5 h.

4. The unit dosage form of claim 2 or 3, wherein the unit dosage form, upon the 14-day oral administration in healthy adult subjects as described in claim 3, provides one or more pharmacokinetics result on post-dosing day 14 selected from:
a mean serum Cₘₐₓ of about 10400 ng/mL;
a mean serum AUC_{τ} of about 32300 ng*h/mL;
a mean serum R_{ac} of about 1.33;
a mean serum Tₘₐₓ of about 1.00 h;
a mean serum t_{1/2} of about 8.76 h;
a mean urine A_{E} of about 82.4 mg;
a mean urine A_{E}/Dose of about 27.5%;
a mean urine CL_{R} of about 44.5 mL/min; and
a mean urine CL_{R}/Fu of about 4838 mL/min.

5. The unit dosage form of any one of claims 2-4, wherein the unit dosage form, upon the 14-day oral administration in healthy adult subjects as described in claim 3, provides one or more pharmacokinetics result on post-dosing day 1 selected from:
a serum Cₘₐₓ of about 8240±about 5390 ng/mL;
a serum AUC_{τ} of about 24900±about 10300 ng*h/mL; and
a serum Tₘₐₓ of about 0.50 to about 4.00 h.

6. The unit dosage form of any one of claims 2-5, wherein the unit dosage form, upon the 14-day oral administration in healthy adult subjects as described in claim 3, provides one or more pharmacokinetics result on post-dosing day 14 selected from:
a serum Cₘₐₓ of about 10400±about 5000 ng/mL;
a serum AUC_{τ} of about 32300±about 11900 ng*h/mL;
a serum R_{ac} of about 1.33±about 0.334;
a serum Tₘₐₓ of about 0.50 to about 4.00 h;
a serum t_{1/2} of about 8.76±about 2.35 h;
a urine A_{E} of about 82.4±about 27.1 mg;
a urine A_{E}/Dose of about 27.5%±about 9.05%;
a urine CL_{R} of about 44.5±about 10.0 mL/min; and
a urine CL_{R}/Fu of about 4838±about 1085 mL/min.

7. The unit dosage form of claim 1, wherein the pharmaceutical composition comprises about 250 mg of grapiprant, or a pharmaceutically acceptable salt thereof, and one or more pharmaceutically acceptable excipient or carrier.

8. The unit dosage form of claim 7, wherein the unit dosage form, upon a 17-day oral administration in elderly subjects with minor renal impairment as follows:
i) one dose in the morning in a fasting state on day 1;
ii) twice daily (BID, approximately every 12 hours) on days 4 through 16: one dose in a fasting state in the morning, and one dose in the evening on an empty stomach;
iii) one dose in the morning in a fasting state on day 17;
provides one or more pharmacokinetics result on post-dosing day 1 selected from:
a mean serum Cₘₐₓ of about 10300 ng/mL;
a mean serum AUC_{τ} of about 30100 ng*h/mL;
a mean serum Tₘₐₓ of about 1.00 h; and
a mean serum T_{1/2} of about 8.63 h.

9. The unit dosage form of claim 7 or 8, wherein the unit dosage form, upon the 17-day oral administration in elderly subjects with minor renal impairment as described in claim 8, provides one or more pharmacokinetics result on post-dosing day 17 selected from:
a mean serum Cₘₐₓ of about 12300 ng/mL;
a mean serum AUC_{τ} of about 40200 ng*h/mL;
a mean serum R_{ac} of about 1.54;
a mean serum Tₘₐₓ of about 1.00 h;
a mean urine A_{E} of about 57.9 mg;
a mean urine A_{E}/Dose of about 23.2%;
a mean urine CL_{R} of about 25.0 mL/min; and
a mean urine CL_{R}/Fu of about 2719 mL/min.

10. The unit dosage form of any one of claims 7-9, wherein the unit dosage form, upon the 17-day oral administration in elderly subjects with minor renal impairment as described in claim 8, provides one or more pharmacokinetics result on post-dosing day 1 selected from:
a serum Cₘₐₓ of about 10300±about 5710 ng/mL;
an AUC_{τ} of about 30100±about 12800 ng*h/mL;
a Tₘₐₓ of about 0.50 to about 1.50 h; and
a serum T_{1/2} of about 8.63±about 2.22 h.

11. The unit dosage form of any one of claims 7-10, wherein the unit dosage form, upon the 17-day oral administration in elderly subjects with minor renal impairment as described in claim 8, provides one or more pharmacokinetics result on post-dosing day 17 selected from:
a serum Cₘₐₓ of about 12300±about 4020 ng/mL;
a serum AUC_{τ} of about 40200±about 17300 ng*h/mL;
a serum R_{ac} of about 1.54±about 0.841;
a serum Tₘₐₓ of about 0.50 to about 1.50 h;
a urine A_{E} of about 57.9±about 18.7 mg;
a urine A_{E}/Dose of about 23.2%±about 7.48%;
a urine CL_{R} of about 25.0±about 4.69 mL/min; and
a urine CL_{R}/Fu of about 2719±about 510 mL/min.

12. The unit dosage form of claim 1, wherein the pharmaceutical composition comprises about 375 mg of grapiprant, or a pharmaceutically acceptable salt thereof, and one or more pharmaceutically acceptable excipient or carrier.

13. The unit dosage form of claim 12, wherein the unit dosage form, upon a single oral administration in a fasted state in healthy adult subjects, provides one or more pharmacokinetics result selected from:
a mean serum AUC (0-tlast) of about 28900 ng*h/mL;
a mean serum AUC (0-inf) of about 29100 ng*h/mL;
a mean serum Cₘₐₓ of about 8990 ng/mL;
a mean serum Tₘₐₓ of about 1 h; and
a mean serum T_{1/2} of about 9.48 h.

14. The unit dosage form of claim 12 or 13, wherein the unit dosage form, upon a single oral administration in a fasted state in healthy adult subjects, provides one or more pharmacokinetics result selected from:
a serum AUC (0-tlast) of about 28900±about 13000 ng*h/mL;
a serum AUC (0-inf) of about 29100±about 13000 ng*h/mL;
a serum Cₘₐₓ of about 8990±about 4270 ng/mL;
a serum Tₘₐₓ of about 0.5 to about 3 h; and
a serum T_{1/2} of about 9.48±about 1.87 h.

15. The unit dosage form of any one of claims 12-14, wherein the unit dosage form, upon a single oral administration in a fed state in healthy adult subjects, provides one or more pharmacokinetics result selected from:
a mean serum AUC (0-tlast) of about 24600 ng*h/mL;
a mean serum AUC (0-inf) of about 24800 ng*h/mL;
a mean serum Cₘₐₓ of about 5840 ng/mL;
a mean serum Tₘₐₓ of about 4 h; and
a mean serum T_{1/2} of about 8.52 h.

16. The unit dosage form of any one of claims 12-15, wherein the unit dosage form, upon a single oral administration in a fed state in healthy adult subjects, provides one or more pharmacokinetics result selected from:
a serum AUC (0-tlast) of about 24600±about 8660 ng*h/mL;
a serum AUC (0-inf) of about 24800±about 8720 ng*h/mL;
a serum Cₘₐₓ of about 5840±about 2890 ng/mL;
a serum Tₘₐₓ of about 1.5 to about 4 h; and
a serum T_{1/2} of about 8.52±about 1.29 h.

17. The unit dosage form of claim 1, wherein the pharmaceutical composition comprises about 150 mg of grapiprant, or a pharmaceutically acceptable salt thereof, and one or more pharmaceutically acceptable excipient or carrier.

18. The unit dosage form of claim 17, wherein the unit dosage form, upon a 14-day oral administration in healthy adult subjects as follows:
i) one dose in the morning in a fasting state on day 1;
ii) twice daily (BID, approximately every 12 hours) on days 2 through 13: one dose in a fasting state in the morning, and one dose in the evening on an empty stomach;
iii) one dose in the morning in a fasting state on day 14;
provides one or more pharmacokinetics result on post-dosing day 1 selected from:
a mean serum Cₘₐₓ of about 2150 ng/mL;
a mean serum AUC_{τ} of about 8970 ng*h/mL; and
a mean serum Tₘₐₓ of about 1.5 h.

19. The unit dosage form of claim 17 or 18, wherein the unit dosage form, upon the 14-day oral administration in healthy adult subjects as described in claim 18, provides one or more pharmacokinetics result on post-dosing day 14 selected from:
a mean serum Cₘₐₓ of about 2670 ng/mL;
a mean serum AUC_{τ} of about 10300 ng*h/mL;
a mean serum R_{ac} of about 1.20;
a mean serum Tₘₐₓ of about 1.50 h;
a mean serum t_{1/2} of about 9.71 h;
a mean urine A_{E} of about 33.3 mg;
a mean urine A_{E}/Dose of about 22.2%;
a mean urine CL_{R} of about 55.2 mL/min; and
a mean urine CL_{R}/Fu of about 6004 mL/min.

20. The unit dosage form of any one of claims 17-19, wherein the unit dosage form, upon the 14-day oral administration in healthy adult subjects as described in claim 18, provides one or more pharmacokinetics result on post-dosing day 1 selected from:
a serum Cₘₐₓ of about 2150±about 1390 ng/mL;
a serum AUC_{τ} of about 8970±about 3880 ng*h/mL; and
a serum Tₘₐₓ of about 1.00 to about 3.00 h.

21. The unit dosage form of any one of claims 17-20, wherein the unit dosage form, upon the 14-day oral administration in healthy adult subjects as described in claim 18, provides one or more pharmacokinetics result on post-dosing day 14 selected from:
a serum Cₘₐₓ of about 2670±about 1160 ng/mL;
a serum AUC_{τ} of about 10300±about 3350 ng*h/mL;
a serum R_{ac} of about 1.20±about 0.165;
a serum Tₘₐₓ of about 0.50 to about 4.00 h;
a serum t_{1/2} of about 9.71±about 1.18 h;
a urine A_{E} of about 33.3±about 11.2 mg;
a urine A_{E}/Dose of about 22.2%±about 7.48%;
a urine CL_{R} of about 55.2±about 12.1 mL/min; and
a urine CL_{R}/Fu of about 6004±about 1313 mL/min.

22. The unit dosage form of claim 1, wherein the pharmaceutical composition comprises about 50 mg of grapiprant, or a pharmaceutically acceptable salt thereof, and one or more pharmaceutically acceptable excipient or carrier.

23. The unit dosage form of claim 22, wherein the unit dosage form, upon a 14-day oral administration in healthy adult subjects as follows:
i) one dose in the morning in a fasting state on day 1;
ii) twice daily (BID, approximately every 12 hours) on days 2 through 13: one dose in a fasting state in the morning, and one dose in the evening on an empty stomach;
iii) one dose in the morning in a fasting state on day 14;
provides one or more pharmacokinetics result on post-dosing day 1 selected from:
a mean serum Cₘₐₓ of about 578 ng/mL;
a mean serum AUC_{τ} of about 3150 ng*h/mL; and
a mean serum Tₘₐₓ of about 1.5 h.

24. The unit dosage form of claim 22 or 23, wherein the unit dosage form, upon the 14-day oral administration in healthy adult subjects as described in claim 23, provides one or more pharmacokinetics result on post-dosing day 14 selected from:
a mean serum Cₘₐₓ of about 611 ng/mL;
a mean serum AUC_{τ} of about 3370 ng*h/mL;
a mean serum R_{ac} of about 1.13;
a mean serum Tₘₐₓ of about 1.25 h;
a mean serum t_{1/2} of about 6.47 h;
a mean urine A_{E} of about 10.6 mg;
a mean urine A_{E}/Dose of about 21.2%;
a mean urine CL_{R} of about 56.3 mL/min; and
a mean urine CL_{R}/Fu of about 6118 mL/min.

25. The unit dosage form of any one of claims 22-24, wherein the unit dosage form, upon the 14-day oral administration in healthy adult subjects as described in claim 23, provides one or more pharmacokinetics result on post-dosing day 1 selected from:
a serum Cₘₐₓ of about 578±about 381 ng/mL;
a serum AUC_{τ} of about 3150±about 1490 ng*h/mL; and
a serum Tₘₐₓ of about 0.50 to about 3.00 h.

26. The unit dosage form of any one of claims 22-25, wherein the unit dosage form, upon the 14-day oral administration in healthy adult subjects as described in claim 23, provides one or more pharmacokinetics result on post-dosing day 14 selected from:
a serum Cₘₐₓ of about 611±about 316 ng/mL;
a serum AUC_{τ} of about 3370±about 1420 ng*h/mL;
a serum R_{ac} of about 1.13±about 0.153;
a serum Tₘₐₓ of about 0.50 to about 3.00 h;
a serum t_{1/2} of about 6.47±about 1.40 h;
a urine A_{E} of about 10.6±about 3.18 mg;
a urine A_{E}/Dose of about 21.2%±about 6.37%;
a urine CL_{R} of about 56.3±about 14.5 mL/min; and
a urine CL_{R}/Fu of about 6118±about 1580 mL/min.

27. The unit dosage form of any one of claims 1-26, wherein the one or more pharmaceutically acceptable excipient or carrier comprises microcrystalline cellulose, lactose monohydrate (modified), croscarmellose sodium, hydroxypropyl cellulose, and magnesium stearate.

28. The unit dosage form of claim 27, wherein the amounts of the excipients per mg of grapiprant are:
about 1.82 mg microcrystalline cellulose;
about 0.88 mg lactose monohydrate (modified);
about 0.2 mg croscarmellose sodium;
about 0.08 mg hydroxypropyl cellulose; and
about 0.02 mg magnesium stearate.

29. The unit dosage form of any one of claims 1-28, wherein the unit dosage form comprises one or more tablets.

30. The unit dosage form of claim 29, wherein one or more tablet comprises about 125 mg of grapiprant, about 227.5 mg of microcrystalline cellulose, about 110.0 mg of lactose monohydrate (modified), about 25.0 mg of croscarmellose sodium, 10.0 mg of hydroxypropyl cellulose, and about 2.5 mg of magnesium stearate.

31. The unit dosage form of claim 29 or 30, wherein one or more tablet comprises about 25 mg of grapiprant, about 45.5 mg of microcrystalline cellulose, about 22.0 mg of lactose monohydrate (modified), about 5.0 mg of croscarmellose sodium, about 2.0 mg of hydroxypropyl cellulose, and about 0.5 mg of magnesium stearate.

32. A method for treating a proliferative disorder in a patient, comprising administering a unit dosage form of any one of claims 1-31.

33. A unit dosage form of a pharmaceutical composition, in oral-powder-for-constitution (OPC) formulation, comprising about 1 mg to about 2000 mg grapiprant, or a pharmaceutically acceptable salt thereof.

34. A unit dosage form of a pharmaceutical composition, in liquid form, comprising about 1 mg to about 2000 mg grapiprant, or a pharmaceutically acceptable salt thereof, and water.

35. The unit dosage form of claim 34 comprising about 1 mg grapiprant, or a pharmaceutically acceptable salt thereof, and water.

36. The unit dosage form of claim 35, upon a single oral administration in a fasted state in healthy adult subjects, provides one or more pharmacokinetics result selected from:
a mean serum Cₘₐₓ of about 6.59 ng/mL;
a mean serum Tₘₐₓ of about 2.5 h; and
a mean serum AUC(Tlast) of about 59.1 ng*h/mL.

37. The unit dosage form of claim 35 or 36, upon a single oral administration in a fasted state in healthy adult subjects, provides one or more pharmacokinetics result selected from:
a serum Cₘₐₓ of about 6.59±about 1.86 ng/mL;
a serum Tₘₐₓ of about 1.5 h to about 8.00 h; and
a serum AUC(Tlast) of about 59.1±about 18.8 ng*h/mL.

38. The unit dosage form of claim 34 comprising about 3 mg grapiprant, or a pharmaceutically acceptable salt thereof, and water.

39. The unit dosage form of claim 38, upon a single oral administration in a fasted state in healthy adult subjects, provides one or more pharmacokinetics result selected from:
a mean serum Cₘₐₓ of about 23.1 ng/mL;
a mean serum Tₘₐₓ of about 3.00 h;
a mean serum AUC(Tlast) of about 246 ng*h/mL;
a mean serum AUC(inf) of about 260 ng*h/mL; and
a mean serum T_{1/2} of about 5.34 h.

40. The unit dosage form of claim 38 or 39, upon a single oral administration in a fasted state in healthy adult subjects, provides one or more pharmacokinetics result selected from:
a serum Cₘₐₓ of about 23.1±about 6.16 ng/mL;
a serum Tₘₐₓ of about 2.50 h to about 6.00 h;
a serum AUC(Tlast) of about 246±about 76.7 ng*h/mL;
a serum AUC(inf) of about 260±about 82.7 ng*h/mL; and
a serum T_{1/2} of about 5.34±about 0.147 h.

41. The unit dosage form of claim 34 comprising about 10 mg grapiprant, or a pharmaceutically acceptable salt thereof, and water.

42. The unit dosage form of claim 41, upon a single oral administration in a fasted state in healthy adult subjects, provides one or more pharmacokinetics result selected from:
a mean serum Cₘₐₓ of about 110 ng/mL;
a mean serum Tₘₐₓ of about 2.00 h;
a mean serum AUC(Tlast) of about 901 ng*h/mL;
a mean serum AUC(inf) of about 917 ng*h/mL; and
a mean serum T_{1/2} of about 5.10 h.

43. The unit dosage form of claim 41 or 42, upon a single oral administration in a fasted state in healthy adult subjects, provides one or more pharmacokinetics result selected from:
a serum Cₘₐₓ of about 110±about 21.0 ng/mL;
a serum Tₘₐₓ of about 1.00 h to about 2.50 h;
a serum AUC(Tlast) of about 901±about 179 ng*h/mL;
a serum AUC(inf) of about 917±about 178 ng*h/mL; and
a serum T_{1/2} of about 5.10±about 1.90 h.

44. The unit dosage form of claim 34 comprising about 30 mg grapiprant, or a pharmaceutically acceptable salt thereof, and water.

45. The unit dosage form of claim 44, upon a single oral administration in a fasted state in healthy adult subjects, provides one or more pharmacokinetics result selected from:
a mean serum Cₘₐₓ of about 262 ng/mL;
a mean serum Tₘₐₓ of about 1.00 h;
a mean serum AUC(Tlast) of about 2120 ng*h/mL;
a mean serum AUC(inf) of about 2150 ng*h/mL; and
a mean serum T_{1/2} of about 6.14 h.

46. The unit dosage form of claim 44 or 45, upon a single oral administration in a fasted state in healthy adult subjects, provides one or more pharmacokinetics result selected from:
a serum Cₘₐₓ of about 262±about 83.1 ng/mL;
a serum Tₘₐₓ of about 1.00 to about 2.00 h;
a serum AUC(Tlast) of about 2120±about 750 ng*h/mL;
a serum AUC(inf) of about 2150±about 740 ng*h/mL; and
a serum T_{1/2} of about 6.14±about 1.36 h.

47. The unit dosage form of claim 34 comprising about 100 mg grapiprant, or a pharmaceutically acceptable salt thereof, and water.

48. The unit dosage form of claim 47, upon a single oral administration in a fasted state in healthy adult subjects, provides one or more pharmacokinetics result selected from:
a mean serum Cₘₐₓ of about 1440 ng/mL;
a mean serum Tₘₐₓ of about 0.75 h;
a mean serum AUC(Tlast) of about 7170 ng*h/mL;
a mean serum AUC(inf) of about 7220 ng*h/mL; and
a mean serum T_{1/2} of about 6.91 h.

49. The unit dosage form of claim 47 or 48, upon a single oral administration in a fasted state in healthy adult subjects, provides one or more pharmacokinetics result selected from:
a serum Cₘₐₓ of about 1440±about 489 ng/mL;
a serum Tₘₐₓ of about 0.50 to about 1.00 h;
a serum AUC(Tlast) of about 7170±about 1720 ng*h/mL;
a serum AUC(inf) of about 7220±about 1720 ng*h/mL; and
a serum T_{1/2} of about 6.91±about 2.57 h.

50. The unit dosage form of claim 34 comprising about 300 mg grapiprant, or a pharmaceutically acceptable salt thereof, and water.

51. The unit dosage form of claim 50, upon a single oral administration in a fasted state in healthy adult subjects, provides one or more pharmacokinetics result selected from:
a mean serum Cₘₐₓ of about 12000 ng/mL;
a mean serum Tₘₐₓ of about 0.75 h;
a mean serum AUC(Tlast) of about 31200 ng*h/mL;
a mean serum AUC(inf) of about 31300 ng*h/mL; and
a mean serum T_{1/2} of about 7.98 h.

52. The unit dosage form of claim 50 or 51, upon a single oral administration in a fasted state in healthy adult subjects, provides one or more pharmacokinetics result selected from:
a serum Cₘₐₓ of about 12000±about 3240 ng/mL;
a serum Tₘₐₓ of about 0.50 to about 1.00 h;
a serum AUC(Tlast) of about 31200±about 7270 ng*h/mL;
a serum AUC(inf) of about 31300±about 7260 ng*h/mL; and
a serum T_{1/2} of about 7.98±about 1.73 h.

53. The unit dosage form of claim 34 comprising about 600 mg grapiprant, or a pharmaceutically acceptable salt thereof, and water.

54. The unit dosage form of claim 53, upon a single oral administration in a fasted state in healthy adult subjects, provides one or more pharmacokinetics result selected from:
a mean serum Cₘₐₓ of about 45000 ng/mL;
a mean serum Tₘₐₓ of about 0.50 h;
a mean serum AUC(Tlast) of about 92200 ng*h/mL;
a mean serum AUC(inf) of about 92500 ng*h/mL; and
a mean serum T_{1/2} of about 8.03 h.

55. The unit dosage form of claim 53 or 54, upon a single oral administration in a fasted state in healthy adult subjects, provides one or more pharmacokinetics result selected from:
a serum Cₘₐₓ of about 45000±about 11500 ng/mL;
a serum Tₘₐₓ of about 0.50 to about 1.00 h;
a serum AUC(Tlast) of about 92200±about 20700 ng*h/mL;
a serum AUC(inf) of about 92500±about 20800 ng*h/mL; and
a serum T_{1/2} of about 8.03±about 1.46 h.

56. The unit dosage form of claim 34 comprising about 1000 mg grapiprant, or a pharmaceutically acceptable salt thereof, and water.

57. The unit dosage form of claim 56, upon a single oral administration in a fasted state in healthy adult subjects, provides one or more pharmacokinetics result selected from:
a mean serum Cₘₐₓ of about 64300 ng/mL;
a mean serum Tₘₐₓ of about 1.00 h;
a mean serum AUC(Tlast) of about 174000 ng*h/mL;
a mean serum AUC(inf) of about 175000 ng*h/mL; and
a mean serum T_{1/2} of about 9.41 h.

58. The unit dosage form of claim 56 or 57, upon a single oral administration in a fasted state in healthy adult subjects, provides one or more pharmacokinetics result selected from:
a serum Cₘₐₓ of about 64300±about 17100 ng/mL;
a serum Tₘₐₓ of about 0.50 to about 1.00 h;
a serum AUC(Tlast) of about 174000±about 56700 ng*h/mL;
a serum AUC(inf) of about 175000±about 56700 ng*h/mL; and
a serum T_{1/2} of about 9.41±about 1.49 h.

59. The unit dosage form of claim 34 comprising about 1500 mg grapiprant, or a pharmaceutically acceptable salt thereof, and water.

60. The unit dosage form of claim 59, upon a single oral administration in a fasted state in healthy adult subjects, provides one or more pharmacokinetics result selected from:
a mean serum Cₘₐₓ of about 83800 ng/mL;
a mean serum Tₘₐₓ of about 0.75 h;
a mean serum AUC(Tlast) of about 242000 ng*h/mL;
a mean serum AUC(inf) of about 242000 ng*h/mL; and
a mean serum T_{1/2} of about 8.13 h.

61. The unit dosage form of claim 59 or 60, upon a single oral administration in a fasted state in healthy adult subjects, provides one or more pharmacokinetics result selected from:
a serum Cₘₐₓ of about 83800±about 21800 ng/mL;
a serum Tₘₐₓ of about 0.50 to about 1.00 h;
a serum AUC(Tlast) of about 242000±about 64000 ng*h/mL;
a serum AUC(inf) of about 242000±about 63900 ng*h/mL; and
a serum T_{1/2} of about 8.13±about 2.18 h.

62. The unit dosage form of claim 34 comprising about 2000 mg grapiprant, or a pharmaceutically acceptable salt thereof, and water.

63. The unit dosage form of claim 62, upon a single oral administration in a fasted state in healthy adult subjects, provides one or more pharmacokinetics result selected from:
a mean serum Cₘₐₓ of about 134000 ng/mL;
a mean serum Tₘₐₓ of about 1.00 h;
a mean serum AUC(Tlast) of about 478000 ng*h/mL;
a mean serum AUC(inf) of about 479000 ng*h/mL; and
a mean serum T_{1/2} of about 8.25 h.

64. The unit dosage form of claim 62 or 63, upon a single oral administration in a fasted state in healthy adult subjects, provides one or more pharmacokinetics result selected from:
a serum Cₘₐₓ of about 134000±about 51600 ng/mL;
a serum Tₘₐₓ of about 0.50 to about 1.00 h;
a serum AUC(Tlast) of about 478000±about 228000 ng*h/mL;
a serum AUC(inf) of about 479000±about 229000 ng*h/mL; and
a serum T_{1/2} of about 8.25±about 1.38 h.

65. The unit dosage form of any one of claims 33-65, further comprising one or more pharmaceutically acceptable excipient or carrier.

66. The unit dosage form of any one of claims 33-65, wherein one or more pharmaceutically acceptable excipient or carrier comprises hydroxypropyl cellulose, microcrystalline cellulose, titanium dioxide, xylitol, and 30% simethicone emulsion.

67. The unit dosage form of claim 66, wherein one or more pharmaceutically acceptable excipient or carrier comprises about 200 mg hydroxypropyl cellulose, about 300 mg microcrystalline cellulose, about 50 mg titanium dioxide, about 400 mg xylitol, and about 33.3 mg 30% simethicone emulsion.

68. The unit dosage form of any one of claims 34-67, wherein the water is about 100 mL.

69. A method for treating a proliferative disorder in a patient, comprising administering a unit dosage form of any one of claims 33-68.
